(19) United States
(12) Patent Application Publication
(10) Pub. No.: US 2016/0090351 A1
(43) Pub. Date: Mar. 31, 2016

(54) INHIBITORS OF DEUBIQUITINATING PROTEASES

(71) Applicant: BRANDEIS UNIVERSITY, Waltham, MA (US)

(72) Inventors: Lizbeth K. Hedstrom, Newton, MA (US); Marcus John Curtis Long, Lockington (GB); Ricky Francis Baggio, Waltham, MA (US); Ann Parrinello Lawson, Sudbury, MA (US)

(21) Appl. No.: 14/785,176

(22) PCT Filed: Apr. 18, 2014
(86) PCT No.: PCT/US14/34655

§ 371 Date: Oct. 16, 2015

Related U.S. Application Data
(60) Provisional application No. 61/813,328, filed on Apr. 18, 2013.

(51) Int. Cl. C07C 211/27; C07C 233/08; C07C 333/08; C07C 271/10

(52) U.S. Cl. CPC C07C 211/27; C07C 271/10; C07C 233/08; C07C 333/08

(57) ABSTRACT

Disclosed are small molecule inhibitors of deubiquitinating enzymes (DUBs), and methods of using them. Certain compounds display a preference for specific ubiquitin specific proteases (USPs).

### RELATED APPLICATIONS

This application claims the benefit of priority to U.S. Provisional Patent Application Ser. No. 61/813,328, filed Apr. 18, 2013; the contents of which are hereby incorporated by reference.

### GOVERNMENT SUPPORT

This invention was made with government support under R01-GM100921 awarded by the National Institutes of Health. The government has certain rights in the invention.

### BACKGROUND OF THE INVENTION

The ubiquitin system is the linchpin in maintenance of cellular fitness. While many studies have focused on ubiquitylation pathways, comparatively little is known about deubquitination proteins (DUBs). DUBs are a large group of proteases that regulate ubiquitin-dependent regulatory pathways by cleaving ubiquitin-protein bonds. DUBs can also cleave C-terminally modified ubiquitin. DUBs are also commonly referred to as deubiquinating proteases, deubiquitylating proteases, deubiquitylating proteinases, deubiquinating proteinases, deubiquitinating peptidases, deubiquitinating isopeptidases, deubiquitylating isozpeptidases, deubiquitinases, deubiquitylases, ubiquitin proteases, ubiquitin hydrolyases, ubiquitin isopeptidases, or DUbs. The human genome encodes in five gene families nearly 100 DUBs with specificity for ubiquitin. Importantly, DUBs may act as negative and positive regulators of the ubiquitin system. In addition to ubiquitin recycling, they are involved in processing of ubiquitin precursors, in proofreading of protein ubiquitination, and in disassembly of inhibitory ubiquitin chains. The term DUBs also commonly refers to proteases that act on ubiquitin-like proteins such as SUMO, NEDD and ISG15. Such DUBs are also known as deSUMOylases, deNEDDylases and delSGylating.

DUBs play several roles in the ubiquitin pathway. First, DUBs carry out activation of ubiquitin and ubiquitin-like proproteins. Second, DUBs recycle ubiquitin and ubiquitin-like proteins that may have been accidentally trapped by the reaction of small cellular nucleophiles with the thiol ester intermediates involved in the ubiquitination of proteins. Third, DUBs reverse the ubiquitination or ubiquitin-like modification of target proteins. Fourth, DUBs are also responsible for the regeneration of monoubiquitin from unanchored polyubiquitin, i.e., free polyubiquitin that is synthesized de novo by the conjugating cellular machinery or that has been released from target proteins by other DUBs. Finally, the deubiquitinating enzymes UCH-L3 and YUH1 are able to hydrolyse mutant ubiquitin UBB+1 despite the fact that the glycine at position 76 is mutated.

One of the main classes of DUBs is cysteine protease DUBs, examples of which include members of the ubiquitin-specific processing protease (USP/UBP) superfamily, and members of the ubiquitin C-terminal hydrolyase (UCH) superfamily. In humans, these proteases are involved in processes including apoptosis, autophagy, cell cycle, DNA repair, chromosome remodelling, transcription, endocytosis, MHC class II immune responses, cytokine responses, oxidative stress response, angiogenesis, metastasis, prohormone processing, and extracellular matrix remodeling important to bone development. Because the ubiquitin pathways are involved in many important physiological processes, the DUBs are potential targets for the treatment of many diseases, including cancer, inflammation, neurodegeneration, and infection.

Cysteine proteases are potential targets for the treatment of many diseases, including inflammation, spinal cord injury, neurodegeneration, autoimmune diseases, infection, and cancer. A general strategy for the design of cysteine protease inhibitors consists of identification of a “warhead” functionality that reacts with the catalytic cysteine, and recognition elements that target specific inhibitors. Most “warheads” are very reactive functionalities, such as Michael acceptors, epoxides and haloketones, that often react nonspecifically with other proteins. There exists a need for new warheads with lower intrinsic activity and the ability to temporarily modify their targets.

Currently-available cell permeable small molecule inhibitors of DUBs, such as G5 and NSC632839, are reactive compounds that irreversibly modify other proteins in addition to DUBs. Many known DUB inhibitors have two reactive sites that will non-specifically cross-link proteins, causing an accumulation of both high molecular weight ubiquitin species and protein aggregates in in vitro assays. So, there exists a need for cell-permeable inhibitors of DUBs or cysteine proteases with lower intrinsic reactivity that react reversibly with proteins, thus increasing their specificity.

### SUMMARY OF THE INVENTION

In certain embodiments, the invention relates to a compound of Formula I:

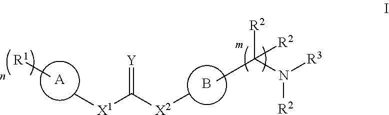

or a pharmaceutically acceptable salt thereof,
wherein, independently for each occurrence,

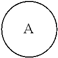

is optionally substituted aryl or optionally substituted heteroaryl;

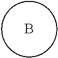

is optionally substituted aryl or optionally substituted heteroaryl;

R¹ is optionally substituted alkyl, halo, —OSO₂R², —OSO₃H, —OC(O)R², —ONO₂, —OP(O)(OR²)₂, alkoxy, or aryloxy;

R² is —H, optionally substituted alkyl, optionally substituted aryl, or optionally substituted heteroaryl;

R³ is —H, optionally substituted alkyl, optionally substituted aryl, optionally substituted heteroaryl, optionally substituted aralkyl, optionally substituted heteroaralkyl, —C(O)R², or —C(O)OR²;

X¹ is O, S, or NR²;

X² is O, S, or NR²;

Y is O, S, or NR²;

n is 0, 1, 2, or 3; and

m is 1, 2, or 3.

In certain embodiments, the invention relates to any one of the aforementioned compounds, provided the compound is not

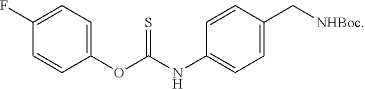 [SMILES: CC(C)(C)OC(=O)NCc1ccc(NC(=S)Oc2ccc(F)cc2)cc1]

In certain embodiments, the invention relates to a compound of Formula II:

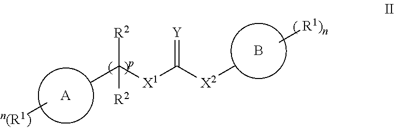

or a pharmaceutically acceptable salt thereof,
wherein, independently for each occurrence,

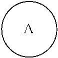

is optionally substituted aryl or optionally substituted heteroaryl;

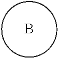

is optionally substituted aryl or optionally substituted heteroaryl;

R¹ is optionally substituted alkyl, halo, —OSO₂R², —OSO₃H, —OC(O)R², —ONO₂, —OP(O)(OR²)₂, alkoxy, or aryloxy;

R² is —H, optionally substituted alkyl, optionally substituted aryl, or optionally substituted heteroaryl;

X¹ is O, S, or NR²;

X² is O, S, or NR²;

Y is O, S, or NR²;

n is 0, 1, 2, or 3; and

p is 0, 1, 2, or 3.

In certain embodiments, the invention relates to any one of the aforementioned compounds, provided the compound is not

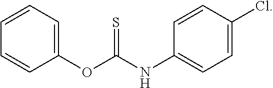 [SMILES: S=C(Nc1ccc(Cl)cc1)Oc1ccccc1]

In certain embodiments, the invention relates to a compound selected from the group consisting of:

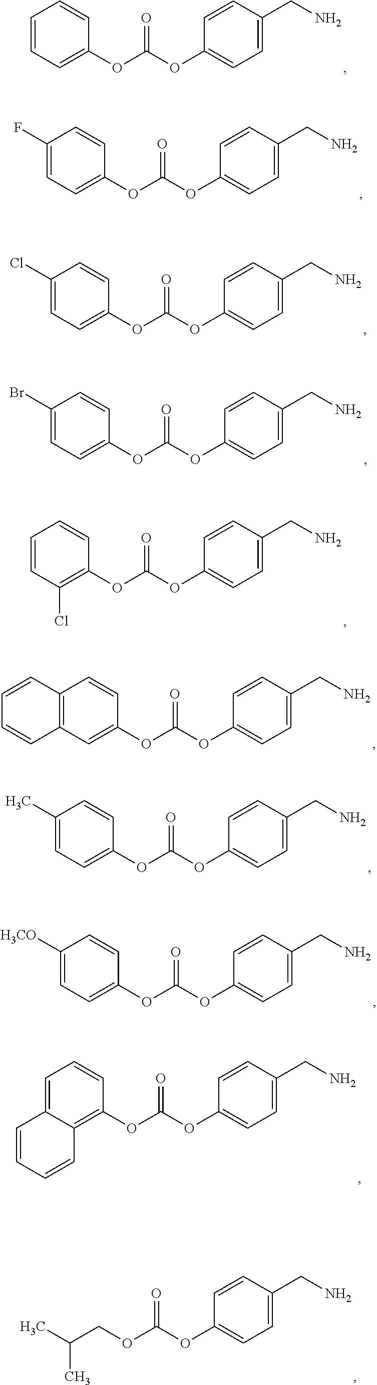

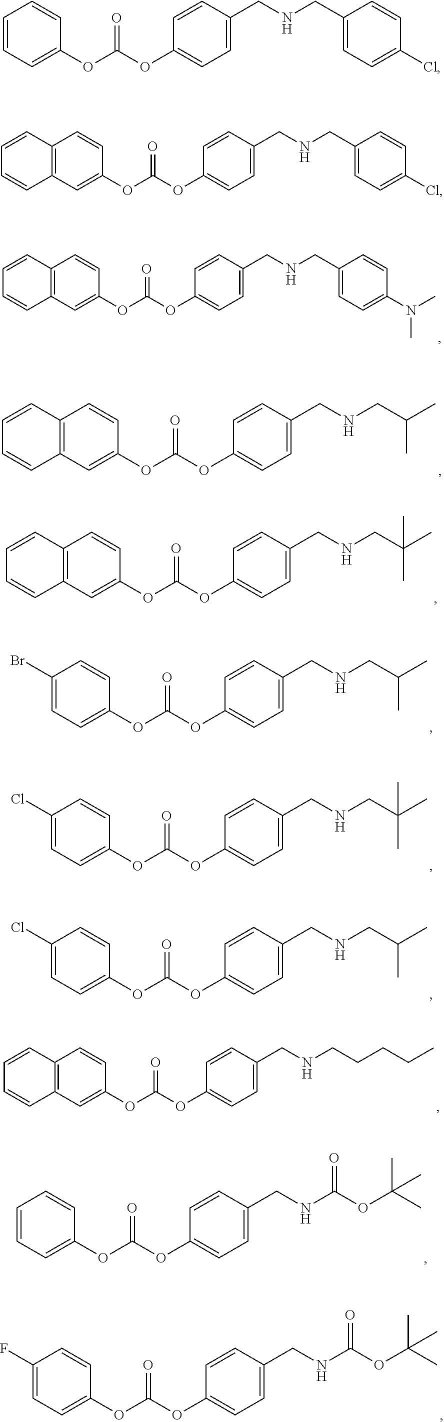

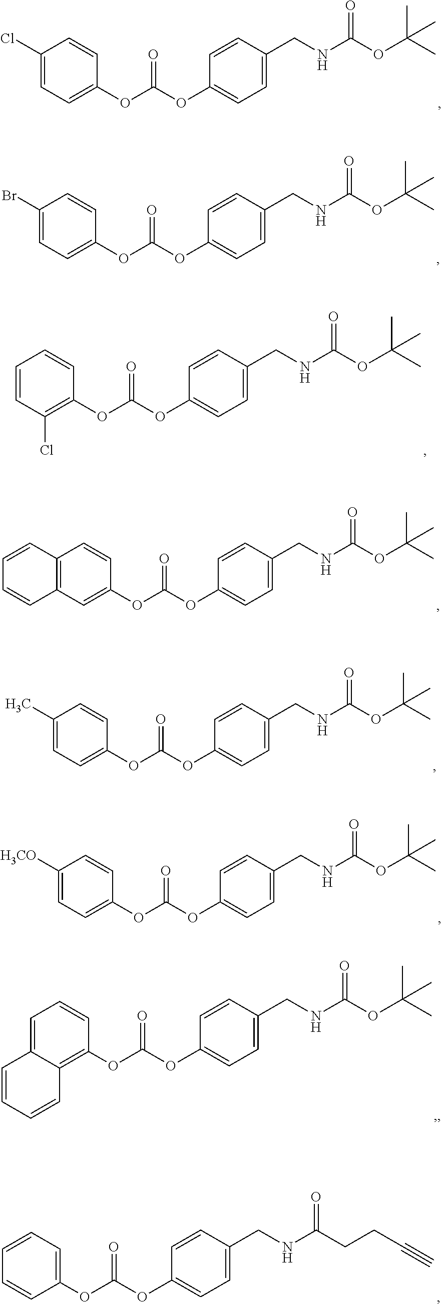

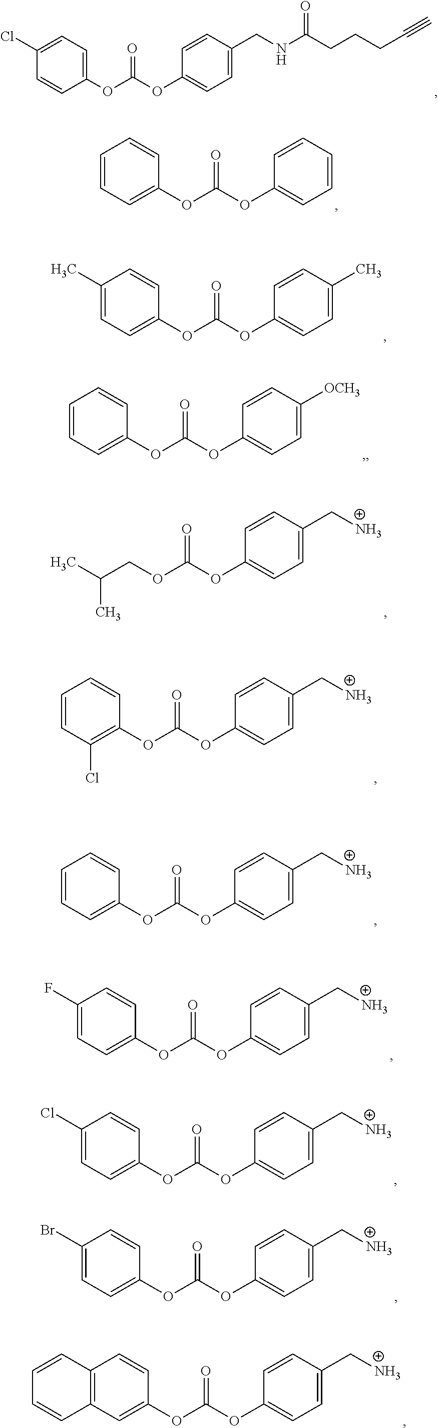

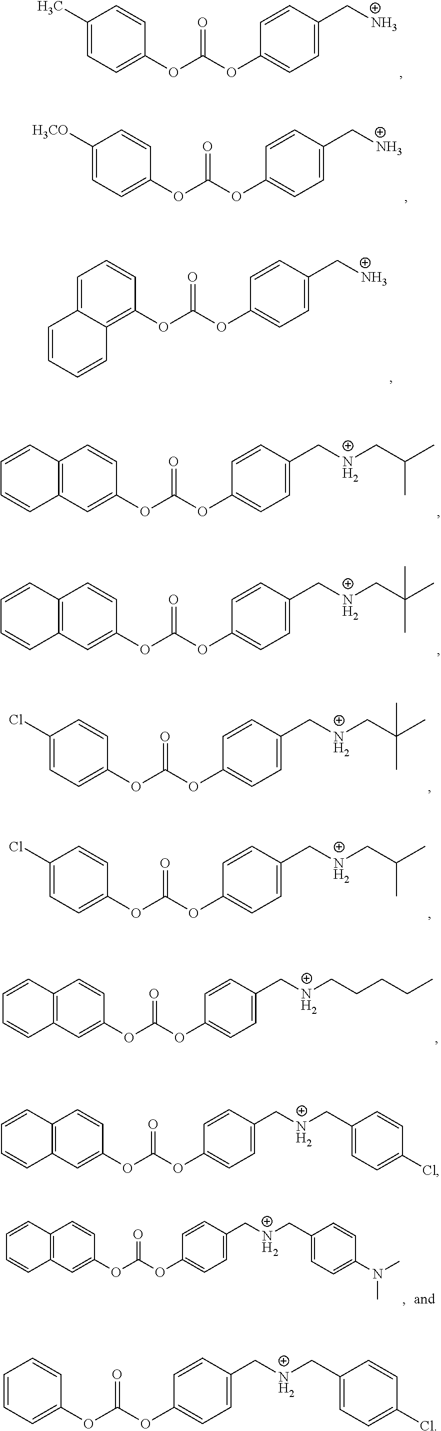

In certain embodiments, the invention relates to a compound selected from the group consisting of:

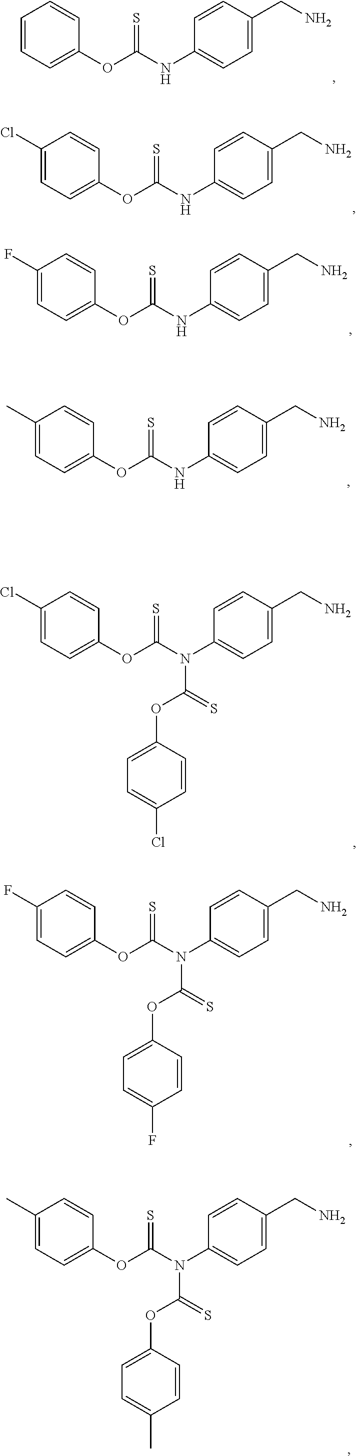

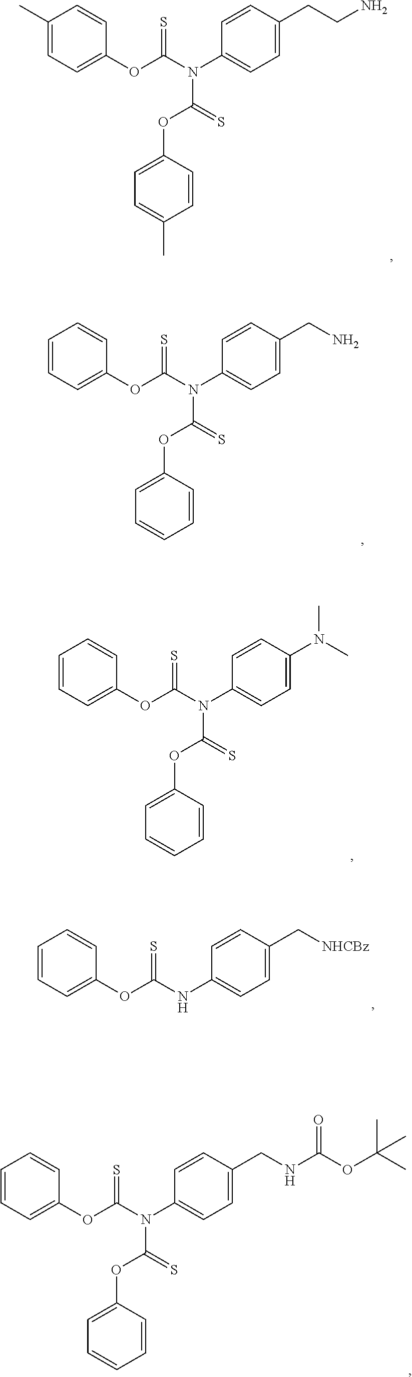

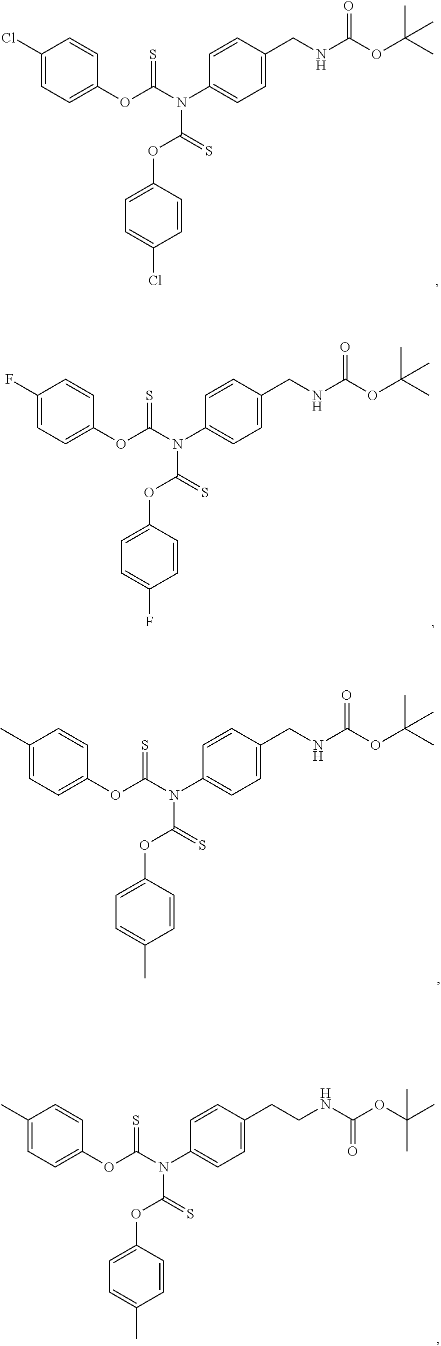

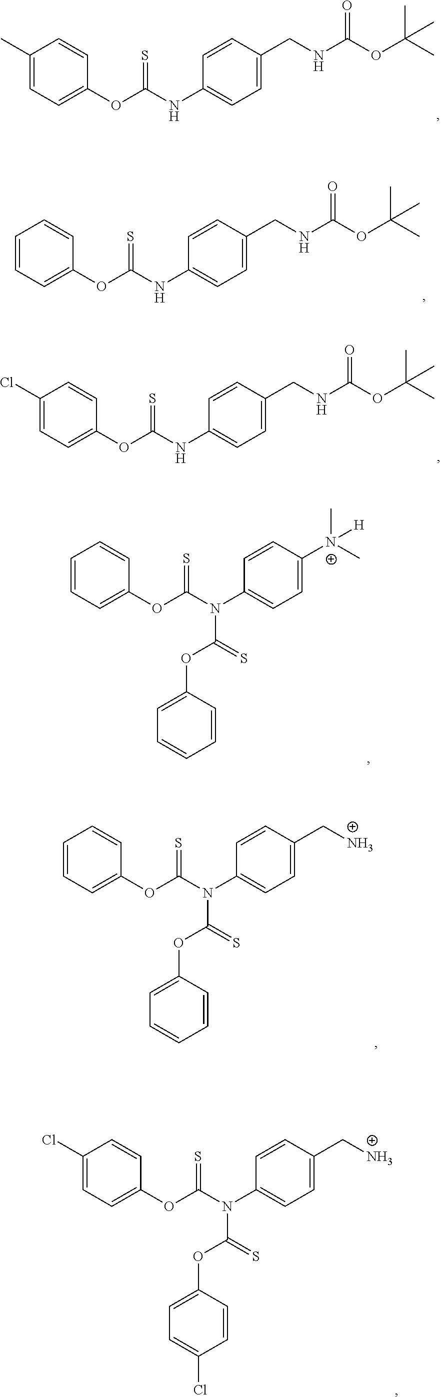 [SMILES: Cc1ccc(OC(=S)Nc2ccc(CNC(=O)OC(C)(C)C)cc2)cc1 ; CC(C)(C)OC(=O)NCc1ccc(NC(=S)Oc2ccccc2)cc1 ; CC(C)(C)OC(=O)NCc1ccc(NC(=S)Oc2ccc(Cl)cc2)cc1 ; C[NH+](C)c1ccc(N(C(=S)Oc2ccccc2)C(=S)Oc2ccccc2)cc1 ; [NH3+]Cc1ccc(N(C(=S)Oc2ccccc2)C(=S)Oc2ccccc2)cc1 ; [NH3+]Cc1ccc(N(C(=S)Oc2ccc(Cl)cc2)C(=S)Oc2ccc(Cl)cc2)cc1]

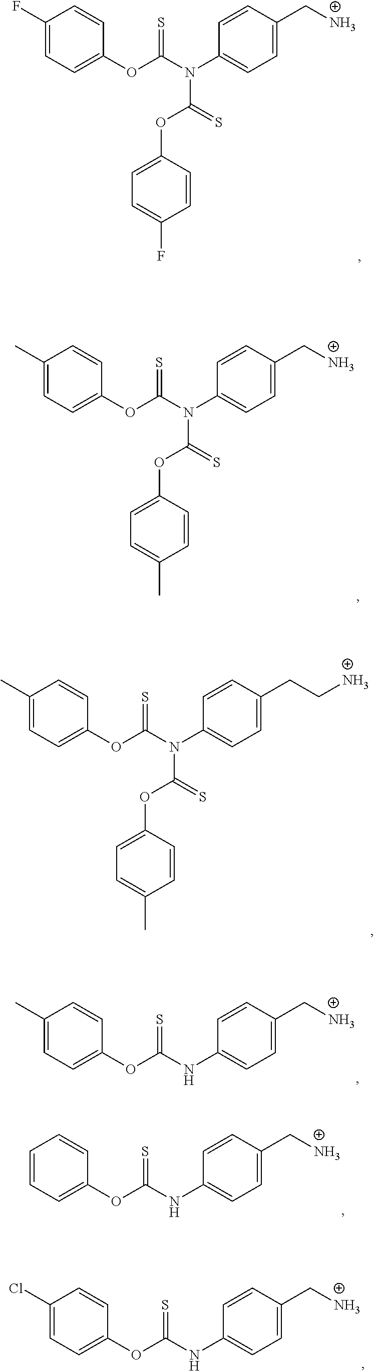 [SMILES: [NH3+]Cc1ccc(N(C(=S)Oc2ccc(F)cc2)C(=S)Oc2ccc(F)cc2)cc1 ; Cc1ccc(OC(=S)N(C(=S)Oc2ccc(C)cc2)c2ccc(C[NH3+])cc2)cc1 ; Cc1ccc(OC(=S)N(C(=S)Oc2ccc(C)cc2)c2ccc(CC[NH3+])cc2)cc1 ; Cc1ccc(OC(=S)Nc2ccc(C[NH3+])cc2)cc1 ; [NH3+]Cc1ccc(NC(=S)Oc2ccccc2)cc1 ; [NH3+]Cc1ccc(NC(=S)Oc2ccc(Cl)cc2)cc1]

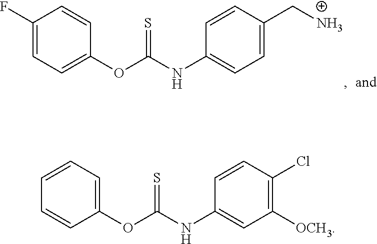 [SMILES: [NH3+]Cc1ccc(NC(=S)Oc2ccc(F)cc2)cc1 ; COc1cc(NC(=S)Oc2ccccc2)ccc1Cl]

In certain embodiments, the invention relates to a compound selected from the group consisting of:

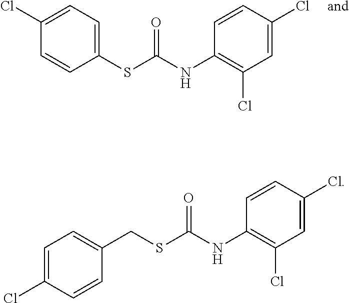 [SMILES: O=C(Nc1ccc(Cl)cc1Cl)Sc1ccc(Cl)cc1 ; "and" ; O=C(Nc1ccc(Cl)cc1Cl)SCc1ccc(Cl)cc1]

In certain embodiments, the invention relates to a compound selected from the group consisting of:

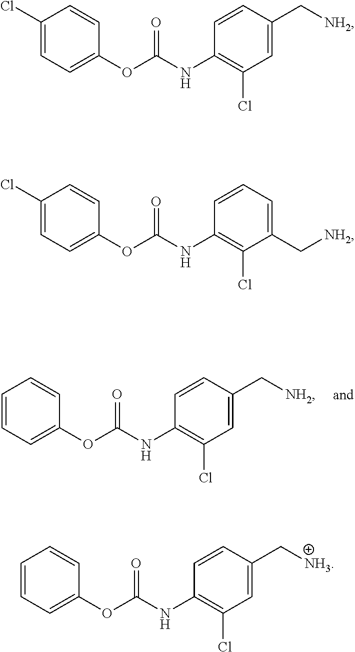

In certain embodiments, the invention relates to a compound selected from the group consisting of:

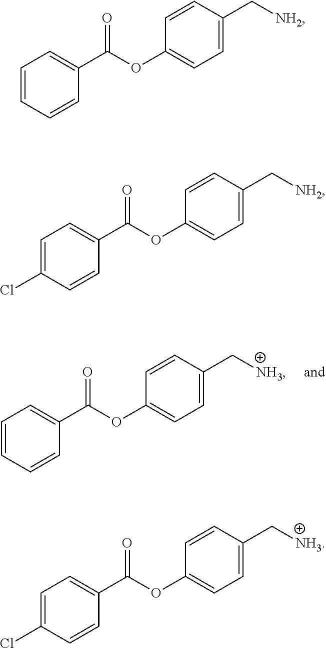

In certain embodiments, the invention relates to a compound selected from the group consisting of:

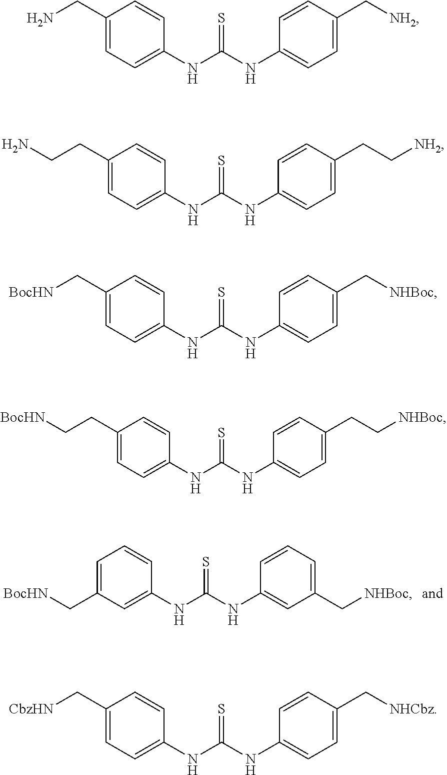

In certain embodiments, the invention relates to a compound of Formula III or Formula IV:

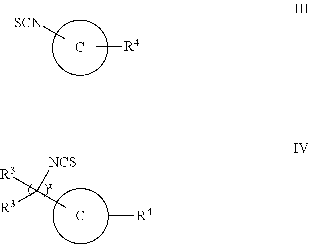

or a pharmaceutically acceptable salt thereof,
wherein, independently for each occurrence,

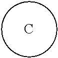

is aryl or heteroaryl;
x is 3, 4, or 5;
R³ is —H, optionally substituted alkyl, optionally substituted aryl, optionally substituted heteroaryl, optionally substituted aralkyl, optionally substituted heteroaralkyl, —C(O)R², or —C(O)OR²;
R² is —H, optionally substituted alkyl, optionally substituted aryl, or optionally substituted heteroaryl; and
R⁴ is absent, or is optionally substituted aminoalkyl, cyano, halo, optionally substituted alkyl, optionally substituted amino, or nitro.

In certain embodiments, the invention relates to any one of the aforementioned compounds, provided the compound is not

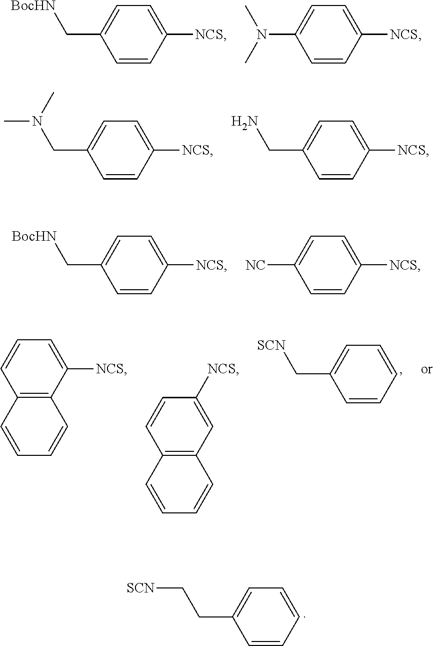

In certain embodiments, the invention relates to a compound selected from the group consisting of:

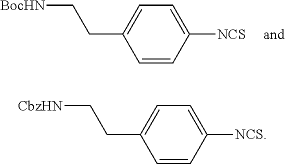

In certain embodiments, the invention relates to a method of preventing or treating a disease in a subject in need thereof comprising the step of: administering to the subject a therapeutically effective amount of any one of the compounds described herein.

In certain embodiments, the invention relates to a method of inhibiting a cysteine protease comprising the step of: contacting the cysteine protease with an effective amount of any one of the compounds described herein.

In certain embodiments, the invention relates to a method of inhibiting a deubiquitinating enzyme comprising the step of: contacting the deubiquitinating enzyme with an effective amount of any one of the compounds described herein.

### BRIEF DESCRIPTION OF THE FIGURES

FIG. 1 is a schematic representation of a hypothetical mechanism of action for the compounds and methods of the invention (X═O or NH; Y═O or S; RE-1 and RE-2=recognition elements that are complementary to the particular target protease). In certain embodiments, the reactivity of RE-1 and RE-2 can be tuned to achieve appropriate rates of acylation and deacylation.

FIG. 2 depicts the structures of exemplary pan-DUB inhibitors of the invention.

FIG. 3 depicts a synthetic route to exemplary compounds of the invention.

FIG. 4 depicts the results of a structure-activity relationship (SAR) assay for various compounds of the invention. (a) Representative blot of lysates treated with compound 4 as defined in FIG. 3. (b) Calculated EC₅₀ (μM) for various compounds (mean and s.e.m., N>2).

FIG. 5 depicts the structures of various compounds of the invention.

FIG. 6 depicts the results of assays for pan-deubiquitinating protease inhibition (compounds numbered as in FIG. 5).

FIG. 7 depicts the results of an inhibition assay for USP9x and USP7.

FIG. 8 depicts the results from a cell permeability assay.

FIG. 9 depicts the results from assays, which indicate that the compounds of the invention do not affect the proteasome or caspases.

FIG. 10 depicts the results from assays of cells treated with a compound of the invention (“compound 4” as defined in FIG. 3).

FIG. 11 depicts the results from assays of cells treated with a compound of the invention (“compound 4” as defined in FIG. 3).

FIG. 12 depicts results indicating that compound 14 (as defined in FIG. 5) inhibits cathepsin C, while compound 3 does not.

FIG. 13 depicts (top) various compounds of the invention, and (middle and bottom) results from assays of cells treated with various compounds of the invention.

FIG. 14 depicts the structures of various compounds of the invention, and compounds used in methods of the invention.

FIG. 15 depicts the structures of various compounds of the invention, and compounds used in methods of the invention.

FIG. 16 tabulates the results of assays measuring the inhibition of human DUBs from HA-Ub-VS assay in K562 lysate (EC₅₀ (μM), after 30 min incubation); NT=not tested.

FIG. 17 tabulates the results of enzyme assays measuring EC₅₀, in μM; NT=not tested.

FIG. 18 tabulates the results of cell assays; NT=not tested.

FIG. 19 tabulates the results of stability assays.

FIG. 20 depicts (top) the effect of selected compounds on deubiquitinating enzymes (DUBs). Cell lysates was treated with 20 μM compound (5 μM WP1130), followed by HA-Ub-VS. DUBs were visualized by immunoblotting for HA. WP1130 and some carbonate compounds are shown for comparison. (bottom) The structures of some compounds are shown.

FIG. 21 depicts results indicating that TU50 selectively inhibits USP9x. HA-Ub-VS is commonly used to label DUBs in cell lysates. 10-12 different DUBs in K562 cell lysates are typically observed by this method. Based on the molecular weight of their respective HA-Ub-VS complexes, these have tentatively been identified as USP9x, USP19, USP7/8, USP28/15, UCHL5 and UCHL3. A. and B. K562 cell lysates were treated with compound for 40 minutes prior to activity labeling with HA-Ub-VS (1.5 μM) for 20 minutes. Samples were analyzed by SDS-PAGE and immunoblotting with anti-HA antibody. C. Quantitation of titrations in A and B. TU49, EC₅₀=3±1 μM; TU50, EC₅₀=3±3 μM; TU46, EC₅₀=11±4 μM; TCM23, EC₅₀>50 μM; TCM28, EC₅₀=45±8 μM. Values were derived by fitting to the in Prism.

FIG. 22 depicts results indicating that TU46, TU49 and TU50 have no effect on proteasome activity. A. Purified 20S proteasome was treated with compound for 30 minutes prior to addition of substrate (Suc-Leu-Tyr-AMC, 100 μM). B. Cos-1 cells expressing with GFP-Ub (G76V) were treated with compound for 8 hours, then the level of GFP was measured by flow cytometry.

FIG. 23 depicts data indicating that TU50 selectively inhibits proliferation of cells that depend on USP9x. Cytoxicity was tested against a panel of cell lines: (i) B16/F10, a metastatic mouse melanoma cell line that suppresses the tumor suppressor Gas1 (Growth arrest-specific 1); (ii) BaF3, an immortalized mouse pro-B cell line that depends on IL-3 for growth and proliferation (L-3 stimulates expression of the pro-survival Bc1-2 family member, Mc1-1; and USP9x is the deubiquitinase responsible for stabilizing Mc1-1); (iii) Cos-1, monkey kidney fibroblast immortalized with SV40 T antigen; (iv) BaF3.p210, BaF3 cells expressing Bcr-Abl kinase (p210), the mutant protein that causes chronic myelogenous leukemia, and the target of Gleevec (The expression of Bcr-Abl cause proliferation to become IL-3-independent. These cells have been widely used in the development of Bcr-Abl kinase inhibitors. USP9x stabilizes Bcr-Abl by removing Ub and blocking degradation via autophagy.); (v) HEK293T, human embryonic kidney cell line expressing SV40 T antigen; (vi) HeLa, human cervical adenocarcinoma cell line; (vii) MCF7, human breast cancer cell line; and (viii) NIH3T3, mouse fibroblast cell line. Of these, the only cells dependent on USP9x for survival are BaF3.p210 and BaF3. A. Cells were treated with a single dose of TU50 for 48 h and viable cells were measured using Alamar Blue®. EC₅₀=8±2 μM for BaF3.p210. B. As above, except that cells were treated with TU50 for 72 h.

FIG. 24 depicts data indicating that TU50 and TU49 induce apoptosis of K562 cells and cause degradation of Bcr-Abl kinase. K562 cells are from a myelogenous leukemia line that expresses Bcr-Abl kinase. A. K562 cells were treated with TU50 for 24 hours after which time the cells were treated with annexin V-FITC and propidium iodide. Cells were then analyzed by flow cytometry. Cells showing only annexin V-FITC were considered apoptotic whereas cells showing both annexin V-FITC and propidium iodide were considered necrotic. N=2. All points show a significant difference to the vehicle (DMSO) with p<0.05. B. K562 cells were treated with TU49 and evaluated as in A. N=2. All points show a significant difference to vehicle (DMSO) with p<0.02. C. Same as A, but the incubation period was 8 hours. N>2. All points show a significant difference to DMSO with p<0.02. D, K562 cells were treated with TU50 for 4 hours. Samples were analyzed by SDS-PAGE and immunoblotting with anti-Bcr-Abl antibody. E. Quantitation of blot in D. The signal for tubulin was used to normalize the Bcr-Abl signal. N=3.

FIG. 25 depicts the results associated with two compounds of the invention (bottom). (top) A BaF3 cell lysate (1.5 mg/mL) was treated with compound for 15 minutes, then with TAMRA-Ub-PA (1 μM) for 20 min. A Typhoon imager was used to scan.

FIG. 26 depicts (A) the structures of compounds screened for cathepsin C inhibition; and (B) EC₅₀ for various compounds after a 30 minute preincubation with compound.

FIG. 27 depicts the structures of various compounds of the invention, and compounds useful in methods of the invention.

FIG. 28 depicts sample inactivation data for compound 13 from FIG. 27. A Cathepsin C was incubated with the compound at varying concentrations for 30 minutes prior to addition of substrate. B as in A but 20 minutes. C as in A but 10 minutes. D as in A but 5 minutes incubation. E plot of the natural log of normalized rate against incubation time. F Replots of k_{obs} against concentration.

FIG. 29 depicts data showing the inhibition of DUBs by diphenyl carbonates. A. Broad spectrum DUB inhibitors. B. Proposed mechanism of inhibition. C. Structures of compounds and values of EC₅₀ for the inhibition of the decomposition of high molecular weight ubiquitinated proteins (HMW-Ub) in lysates prepared from HEK 293T cells expressing HA-Ub. The values of EC₅₀ are the mean±s.e.m. of at least 3 independent experiments as in FIG. 30. Brackets denote the values of EC₅₀ for the inhibition of the decomposition of HMW-Ub in lysates prepared from Cos-1 cells expressing HA-Ub.

FIG. 30 depicts data showing that diphenylcarbonates inhibit the decomposition of high molecular weight ubiquitinated proteins (HMW-Ub). Lysates were prepared from HEK 293T cells expressing HA-ubiquitin. Samples were incubated at 37° C. and reactions were quenched by the addition of reducing Laemmli buffer. HMW-Ub was assessed by SDS-PAGE and immunoblotting with anti-HA antibody. A. Representative immunoblots measuring the decomposition of HMW-Ub in lysates treated with either the DMSO vehicle, G5 (10 μM) or C4 (500 μM). B. Plot of the decomposition of HMW-Ub in lysates treated with DMSO and C4 (500 μM) (N=2; error bars denote range). C. Inhibition of HMW-Ub decomposition by diphenylcarbonates. Lysates were treated with diphenylcarbonates (50 μM). “-” denotes no treatment; D, 1% DMSO vehicle; IU1, an USP14-specific inhibitor; Bort, bortezomib, a proteasome inhibitor. D. Representative decomposition of HMW-Ub after 2 h incubation in the presence of varying concentrations of C4. See also FIG. 36. E. Quantitation of D. The values of EC₅₀ reported in FIG. 29B are the average and S.E.M. of at least 3 independent experiments. See also FIG. 37 and FIG. 38.

FIG. 31 depicts data showing that diphenyl carbonates are broad spectrum DUB inhibitors with selectivity for USPs. A. A lysate of HEK 293T cells (1.5 mg/mL) was treated with diphenylcarbonates (75 μM) for 30 minutes prior to addition of HA-Ub-VS (1.5 μM). B. HEK 293T cell lysate was treated with C17 for 30 minutes prior to addition of HA-Ub-VS. C. A lysate of HEK 293T cells (15 mg/mL) was treated with either C17 (250 μM) or DMSO for 30 minutes. After this time lysate was diluted ten-fold and HA-Ub-VS (1.5 μM) was added. Aliquots were removed at the stated time points and analyzed by HA blot. D. A lysate of HEK 293T cells (1.5 mg/mL) was treated with C17 (25 μM) or DMSO immediately followed by HA-Ub-VS (1.5 μM). Aliquots were removed at the stated time points and analyzed for HA. See also FIG. 38.

FIG. 32 depicts data showing that diphenyl carbonates inhibit DUBs in HEK 293T cells. A. Viability of HEK 293T cells assessed by the propidium iodide exclusion method: diphenyl carbonate (100 μM), bortezomib (V, 20 μM) and G5 (2 μM). N≧3, mean+/−s.d. B. HEK 293T cells were treated with the stated compounds (50 μM) for 2 h and then assayed for the accumulation of K48-linked Ub species. C. As in A, but K63-linked Ub was assayed. D. HEK 293T cells were treated with C14, C15, C17, C18 (100 μM) or DMSO for 2 hours, then harvested, lysed and treated with HA-Ub-VS. After 30 min, lysates were analyzed by SDS-PAGE and probed for HA, tubulin and actin. An intervening lane was removed for clarity. E. HEK 293T cells expressing Ub-G76V-GFP were treated with diphenyl carbonates (100 μM), bortezomib (20 μM) and G5 (2 μM). Only bortezomib treatment caused an increase in GFP fluorescence. N>3, Mean+/−s.d. See also FIG. 39.

FIG. 33 depicts data showing that C15 causes the accumulation of soluble HMW-Ub in Cos-1 cells. A. Cos-1 cells were treated with C15 dosing every 2 h for 4 hours. Cells were lysed in standard lysis buffer (without detergent) and the sample was clarified prior to analysis. The accumulation of K48-linked ubiquitin was assayed by SDS-PAGE and by western blot. An intervening lane has been removed for clarity. B Lysates and pellet in A were sonicated in SDS at 4° C., centrifuged then analyzed by SDS-PAGE and by western blot. An intervening lane has been removed for clarity. C. Quantitation of blots in A. D. Quantitation of blots in B.

FIG. 34 depicts data showing that diphenylcarbonates cause the accumulation of HMW-Ub and reduce the levels of Bcr-Abl in K562 cells. A. K562 cells were treated with diphenyl carbonates (50 μM) for 2 h and then assayed for the accumulation of K48-linked Ub. B. As in A, but Bcr-Abl was measured by immunoblotting with anti-Abl antibodies. C. K562 cells were treated with C17 (50 μM) in the presence and absence of bortezomib (6 μM) for 4 hours and Bcr-Abl was measured by immunblotting with anti-Abl antibodies. D. Quantitation of blot in C. Significance: DMSO relative to C17 and bortezomib p=0.002; DMSO relative to C17, p=0.03. E. K562 cells were treated with C15 for 4 hours then analyzed for SMAD4 monoubiquitination by western blot. F. Quantitation of blots in A. (N=3). See also FIG. 40.

FIG. 35 depicts data showing that diphenylcarbonates reduce the levels of Mdm2 and cause the accumulation of P53 and P21 in MCF7 cells. A. MCF7 cells were treated with C17 for 4 h and Mdm2 levels were measured. B. As in A, P53 measured. C. As in A, P21 measured. D. MCF7 and B16/F10 cells were treated with C17 every 24 hours. After 72 hours, the number of viable cells was measured by Alamar Blue®. See also FIG. 41 and FIG. 42.

FIG. 36 depicts data showing that diphenyl carbonates inhibit DUBs. A. A representative experiment. Lysates were prepared from HEK 293T cells expressing HA-ubiquitin and treated with vehicle alone (DMSO, final concentration 1%), or compound (concentrations shown in C). Samples were incubated at 37° C. and analyzed by SDS-PAGE and immunoblotting with anti-HA antibody. Intervening lanes have been removed for clarity. D=DMSO, Bort=bortezomib (a proteasome inhibitor); LDN=LDN 54777 (a wDUB inhibitor); NSC=NSC 632839 (a broad spectrum DUB inhibitor); G5=G5 isopeptidase inhibitor 1 (a broad spectrum DUB inhibitor); B. Conditions as in A, concentrations shown in C. IU1 (a specific USP14 inhibitor); UbA1=ubiquitin aldehyde (a broad spectrum DUB inhibitor). C. Quantification of blots as in A and B, relative to the control (vehicle alone at time=0). N=2, average and range are shown. D. Conditions as in A. E-I Lysates were prepared from HEK 293T cells expressing HA-Ub and treated with either vehicle alone (1% DMSO) or compound at 37° C. Samples were analyzed by SDS-PAGE and immunoblotting with anti-HA antibody. E. Treatment with C4 and C14 after 3 h. F. Comparison of C4 and C14 after incubation for 6 h. G. Treatment with C13 and C17 after incubation for 3 h. H. Treatment with C13 and C17 after incubation for 3 h. I. Treatment with C3 and C17 after incubation for 3 h. In all instances, “-” indicates no treatment.

FIG. 37 depicts data showing that diphenyl carbonates cause the accumulation of K48-linked HMW-Ub in wild-type HEK 293T cells but do not affect deSUMOylation or inhibit representative cysteine proteases. Untransfected HEK 293T cell lysates were treated with the vehicle alone (DMSO at 1%) or compound and incubated at 37° C. for 2 h. Samples were analyzed by SDS-PAGE and immunoblotting with antibody recognizing K48-linked ubiquitin. D=DMSO; G5=G5 isopeptidase inhibitor 1. A-D show titrations of different compounds (see FIG. 29B for structures). E-H Lysates were prepared from HEK 293T cells expressing HA-SUMO and treated with vehicle alone (DMSO, 1%) or compound. Samples were incubated for the appropriate time and analyzed by SDS-PAGE and immunoblotting with anti-HA antibody. Representative experiments are shown. E. Vehicle control. F. G5, 5 μM; G. C4, 300 μM. H. Quantification of blots as in E-G, N=2 average and range. I-J. Lysates were prepared from HEK 293T cells expressing HA-Ub and treated with either vehicle alone (1% DMSO) or compound at 37° C. for 3 h. Samples were analyzed by SDS-PAGE and immunoblotting with anti-HA antibody. I. A representative experiment. J. Quantitation of experiments in I (N=2 average and range shown). In all instances, “-” indicates no treatment. K. Ficin was preincubated with inhibitor (100 μM) for 30 min prior to addition of Z-Arg-AMC (300 μM). L. Papain was preincubated with inhibitor (100 μM) for 30 min prior to addition of Z-Arg-AMC (300 μM).

FIG. 38 depicts data showing that diphenyl carbonates inhibit HA-Ub-VS labeling in cell lysates. Lysates (1 mg/mL) were treated with varying concentrations of compounds for 30 minutes prior to addition of HA-Ub-VS (1.5 μM). A. HEK 293T cell lysates. B. Cos-1 cell lysates. C. HEK 293T lysates. D. HEK 293T lysates. E. HEK 293T lysates. F-G. HEK 293T Lysates (10 mg/mL) were treated with varying concentrations of compounds for 10 minutes prior to addition of HA-Ub-VS (1.5 μM) for 20 minutes. Lysate was diluted 10-fold prior to addition of 1.5× Laemelli loading buffer. F. Lysates were blotted for USP7. G. Lysates were blotted for USP15.

FIG. 39 depicts data showing the effects of diphenyl carbonates in whole cells. A. Cos-1 cells expressing GFP-Ub(G76V) were treated with the stated diphenyl carbonate (100 μM), bortezomib (B, 20 μM) or DMSO (D) for 8 h. GFP levels were quantified by flow cytometry. B. Cells from A were assessed for viability using propidium iodide dye exclusion assay. C. CHO cells expressing GFP-ubiquitin (G76V) were treated with the stated diphenyl carbonate (100 μM), bortezomib (B, 20 μM), G5 (10 μM) or DMSO for 8 hours. After this time GFP levels were quantified by flow cytometry. D. Cells from C were assessed for viability using propidium iodide dye exclusion. E. MCF7 cells were treated with C15 (100 μM) dosing every 2 h. The accumulation of K48-linked ubiquitin was assayed by SDS-PAGE and by western blot. F. MCF7 cells were treated as in A but the accumulation of K63-linked ubiquitin was assayed. G. Cos-1 cells were treated with C15 for 2 hours then analyzed for K48-linked ubiquitin by western blot. H. Similar experiment to C, but K63-linked ubiquitin was assayed. M, markers. I. Quantitation of blots in D (N=3).

FIG. 40 depicts data showing that diphenyl carbonates induce the degradation of Bcr-Abl kinase. A. K562 cells were treated with C17 for 24 h (1 dose) then analyzed for Bcr-Abl expression by western blot with anti-Abl antibody. B. Quantitation of blots in A normalized to actin. C. K562 cells were treated with C17 for 24 h then the amount of cells in G1 and apoptosis were recorded (N=3). D. K562 were treated with C15 for 4 h then analyzed for Bcr-Abl expression using western blot. E. Quantitation of Bcr-Abl from D normalized to tubulin (N=4). F. Same K562 lysates were separately analyzed for K63-linked ubiquitin. G. Quantitation of blots in F (N=4). Differences between all bars is significant p<0.01). H. K562 cells were treated with C15 for 24 h after which time cells were fixed in ethanol, treated with propodium iodide/RNAse then analyzed by FACS. Viability was calculated using forward and side scatter. I. Same experiment as H but red fluorescence was measured to determine DNA content

FIG. 41 depicts data showing that compound C17 and C15 increase P53 expression and upregulate K48-linked Ub in MCF7 cells. A. MCF7 cells were treated with C17 (50 μM) for 48 h, dosed every 24 h and samples were analyzed for K48-linked ubiquitin. Note that samples were not sonicated, so only soluble HMW-Ub is recovered. B. A similar experiment to A but samples were analyzed for p53. C. Quantitation of blots in B. Each point has a P<0.001 for an increase relative to the DMSO vehicle (N=2). D. MCF7 cells were treated with C15 (100 μM) dosing every 2 h. The accumulation of K48-linked ubiquitin was assayed by SDS-PAGE and by western blot. E. MCF7 cells were treated as in D but the accumulation of K63-linked ubiquitin was assayed. F. Cos-1 cells were treated with C15 for 2 hours then analyzed for K48-linked ubiquitin by western blot. G. Similar experiment to F, but K63-linked ubiquitin was assayed. H. Quantitation of blots in G (N=3).

FIG. 42 depicts data showing the effects of Compound C15 on MCF7 cells. MCF7 cells were dosed every 2 hours with the indicated concentration of C15. After 6 hours, cells were lysed by freeze thaw and then sonicated in 0.5% SDS. A. Total lysates were blotted for K48-linked ubiquitin and actin. B. Normalized K48-linked ubiquitin signal was plotted as a function of concentration of C15 (N=2). C. Effect of C15 on PARP cleavage and P53 levels. Doxorubicin (Dox) is a DNA damaging agent that serves as a positive control for the induction of P53. D. Quantitation of P53 levels in C relative to actin (N=2). E. Effects of C15 on Mdm2 and P21 levels. MCF7 cells were treated under the stated conditions then analyzed for cell cycle. F. DMSO treated cells. G. Cells treated with C17 (25 μM), dosed every 24 hours. H. Cells treated with C17 (50 μM), dosed every 24 hours. I. Quantitation of graphs in F, G, H. J. MCF7 cells were plated together with 2-naphthol, 4-(aminomethyl)phenol or C17 at the stated concentration. These cells were left for 72 h (single dose) after which time the total number of viable cells was analyzed using Alamar Blue®. All data are n≧4 showing mean+/−s.e.m. K. MCF7 cells were plated with C17 for 24 hours after which time the total number of viable cells was measured by Alamar Blue®. L. B16/F10 cells were treated with DMSO, C17 or P005091 for 72 h. C17 was dosed every 24 h. After this time, cells were analyzed by flow cytometry.

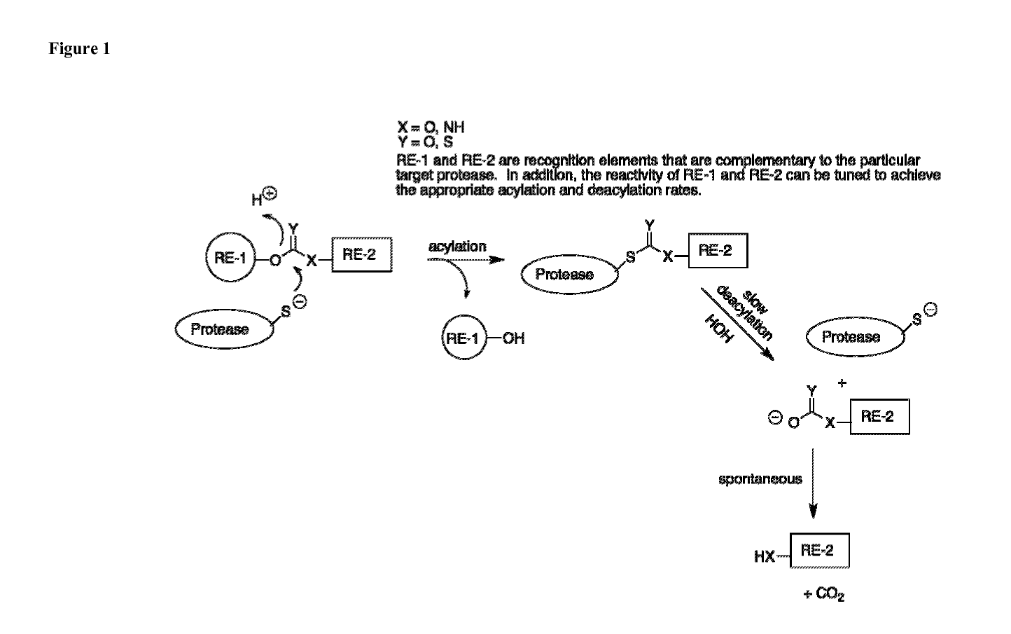

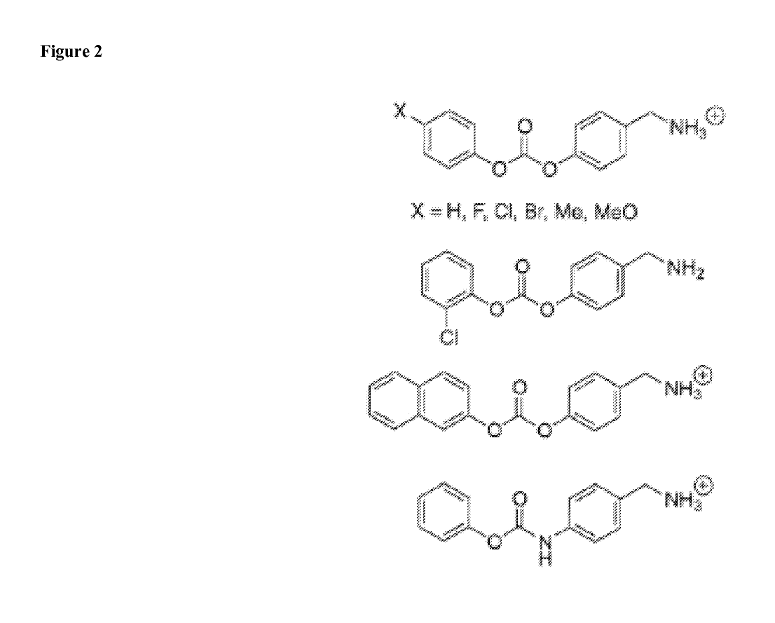

### DETAILED DESCRIPTION OF THE INVENTION

### Overview

In certain embodiments, the invention relates to compounds comprising a simple, readily modified pharmacophore that inhibits DUBs (i.e., deubquitination proteins or deubquitination proteases). In certain embodiments, the compounds do not comprise a highly reactive electrophile. In certain embodiments, the compounds are selective; that is, the compounds do not significantly or substantially affect the proteasome or caspases. In certain embodiments, the compounds are substantially cell permeable. In certain embodiments, the compounds are effective in a wide range of cell lines.

In certain embodiments, the invention relates to a method of inhibiting a DUB in a cell comprising contacting the cell with a compound of the invention. In certain embodiments, the methods of the invention result in an accumulation of high molecular weight ubiquitin species. In certain embodiments, the methods of the invention do not result in any substantial accumulation of other protein aggregates.

Because of their mechanism of action, in certain embodiments, these compounds may also inhibit other cysteine proteases, including cathepsin C, caspases, and viral proteases. Cysteine proteases regulate many important physiological processes, and are potential targets for the treatment of many diseases, including inflammation, arthritis, osteoporosis, gingivitis, cancer, neurodegeneration, and infection.

In addition, in certain embodiments, the compounds of the invention are useful in methods of investigating protein modification pathways, such as the ubiquitin pathway, the SUMO pathway, or the Nedd pathway.

In certain embodiments, the invention relates to a diphenylcarbonate that acts as a slow DUB substrate, so inhibition is transient nature, which may mitigate off-target effects and could be responsible for lower toxicity than known compounds. Diphenylcarbonates are potent inhibitors of USPs than UCH-Ls. This selectivity appears to derive from the stability of the thiocarbonylated enzyme.

In certain embodiments, treatment of MCF7 cells with a compound of the invention elicits P53 up regulation, which ultimately leads to apoptosis. In certain embodiments, the compounds of the invention also cause degradation of Bcr-Abl kinase and increased monoubiquitination of SMAD4, as expected when USP9x is inhibited. In certain embodiments, the compounds of the invention do not induce the accumulation of insoluble ubiquitin aggregates even at high concentrations.

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer.

“Cycloalkyl” means mono- or bicyclic or bridged saturated carbocyclic rings, each having from 3 to 12 carbon atoms. Likewise, preferred cycloalkyls have from 5-12 carbon atoms in their ring structure, and more preferably have 6-10 carbons in the ring structure.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkenyl, and —(S)—(CH₂)ₘ—R¹, wherein m and R¹ are defined below. Representative alkylthio groups include methylthio, ethylthio, and the like.

The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O— alkenyl, —O-alkynyl, —O—(CH₂)ₘ—R¹, where m and R₁ are described below.

The terms “amine” and “amino” are art-recognized and refer to both unsubstituted and substituted amines, e.g., a moiety that can be represented by the formulae:

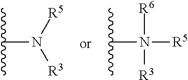

wherein R³, R⁵ and R⁶ each independently represent a hydrogen, an alkyl, an alkenyl, —(CH₂)ₘ—R¹, or R³ and R⁵ taken together with the N atom to which they are attached complete a heterocycle having from 4 to 8 atoms in the ring structure; R¹ Represents an alkenyl, aryl, cycloalkyl, a cycloalkenyl, a heterocyclyl, or a polycyclyl; and m is zero or an integer in the range of 1 to 8. In certain embodiments, only one of R³ or R⁵ can be a carbonyl, e.g., R³, R⁵, and the nitrogen together do not form an imide. In even more certain embodiments, R³ and R⁵ (and optionally R⁶) each independently represent a hydrogen, an alkyl, an alkenyl, or —(CH₂)ₘ—R¹. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 6- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like.

The terms “heterocyclyl” or “heterocyclic group” refer to 3- to 12-membered ring structures, more preferably 5- to 12-membered rings, more preferably 6- to 10-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “carbonyl” is art-recognized and includes such moieties as can be represented by the formula:

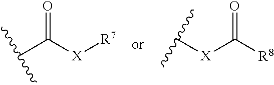

wherein X is a bond or represents an oxygen or a sulfur, and R⁷ represents a hydrogen, an alkyl, an alkenyl, —(CH₂)ₘ—R¹ or a pharmaceutically acceptable salt, R⁸ represents a hydrogen, an alkyl, an alkenyl or —(CH₂)ₘ—R¹, where m and R¹ are as defined above. Where X is an oxygen and R⁷ or R⁸ is not hydrogen, the formula represents an “ester.” Where X is an oxygen, and R⁷ is as defined above, the moiety is referred to herein as a carboxyl group, and particularly when R⁷ is a hydrogen, the formula represents a “carboxylic acid”. Where X is an oxygen, and R⁸ is a hydrogen, the formula represents a “formate.” In general, where the oxygen atom of the above formula is replaced by a sulfur, the formula represents a “thiocarbonyl” group. Where X is a sulfur and R⁷ or R⁸ is not hydrogen, the formula represents a “thioester” group. Where X is a sulfur and R⁷ is a hydrogen, the formula represents a “thiocarboxylic acid” group. Where X is a sulfur and R⁸ is a hydrogen, the formula represents a “thioformate” group. On the other hand, where X is a bond, and R⁷ is not hydrogen, the above formula represents a “ketone” group. Where X is a bond, and R⁷ is a hydrogen, the above formula represents an “aldehyde” group.

The term “thioxamide,” as used herein, refers to a moiety that can be represented by the formula:

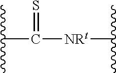

in which R^{t} is selected from the group consisting of the group consisting of hydrogen, alkyl, cycloalkyl, aralkyl, or aryl, preferably hydrogen or alkyl. Moreover, “thioxamide-derived” compounds or “thioxamide analogs” refer to compounds in which one or more amide groups have been replaced by one or more corresponding thioxamide groups. Thioxamides are also referred to in the art as “thioamides.”

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The term “sulfamoyl” is art-recognized and includes a moiety that can be represented by the formula:

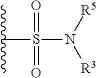

in which R³ and R⁵ are as defined above.

The term “sulfate” is art recognized and includes a moiety that can be represented by the formula:

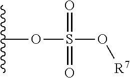

in which R⁷ is as defined above.

The term “sulfonamide” is art recognized and includes a moiety that can be represented by the formula:

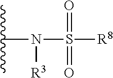

in which R³ and R⁸ are as defined above.

The term “sulfonate” is art-recognized and includes a moiety that can be represented by the formula:

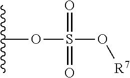

in which R⁷ is an electron pair, hydrogen, alkyl, cycloalkyl, or aryl.

The terms “sulfoxido” or “sulfinyl”, as used herein, refers to a moiety that can be represented by the formula:

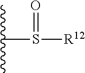

in which R¹² is selected from the group consisting of the group consisting of hydrogen, alkyl, alkenyl, alkynyl, cycloalkyl, heterocyclyl, aralkyl, or aryl.

As used herein, the definition of each expression, e.g., alkyl, m, n, etc., when it occurs more than once in any structure, is intended to be independent of its definition elsewhere in the same structure.

For purposes of this invention, the chemical elements are identified in accordance with the Periodic Table of the Elements, CAS version, Handbook of Chemistry and Physics, 67th Ed., 1986-87, inside cover.

### EXEMPLARY COMPOUNDS OF THE INVENTION

In certain embodiments, the invention relates to a compound of Formula I:

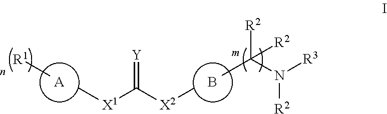

or a pharmaceutically acceptable salt thereof,
wherein, independently for each occurrence,

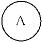

is optionally substituted aryl or optionally substituted heteroaryl;

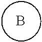

is optionally substituted aryl or optionally substituted heteroaryl;

R¹ is optionally substituted alkyl, halo, —OSO₂R², —OSO₃H, —OC(O)R², —ONO₂, —OP(O)(OR²)₂, alkoxy, or aryloxy;

R² is —H, optionally substituted alkyl, optionally substituted aryl, or optionally substituted heteroaryl;

R³ is —H, optionally substituted alkyl, optionally substituted aryl, optionally substituted heteroaryl, optionally substituted aralkyl, optionally substituted heteroaralkyl, —C(O)R², or —C(O)OR²;

X¹ is O, S, or NR²;

X² is O, S, or NR²;

Y is O, S, or NR²;

n is 0, 1, 2, or 3; and

m is 1, 2, or 3.

In certain embodiments, the invention relates to any one of the aforementioned compounds, provided the compound is not

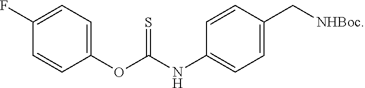 [SMILES: CC(C)(C)OC(=O)NCc1ccc(NC(=S)Oc2ccc(F)cc2)cc1]

In certain embodiments, the invention relates to a compound of Formula II:

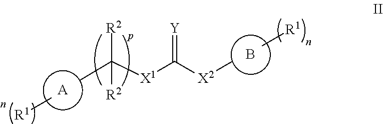

or a pharmaceutically acceptable salt thereof,
wherein, independently for each occurrence,

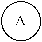

is optionally substituted aryl or optionally substituted heteroaryl;

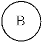

is optionally substituted aryl or optionally substituted heteroaryl;

R¹ is optionally substituted alkyl, halo, —OSO₂R², —OSO₃H, —OC(O)R², —ONO₂, —OP(O)(OR²)₂, alkoxy, or aryloxy;

R² is —H, optionally substituted alkyl, optionally substituted aryl, or optionally substituted heteroaryl;

X¹ is O, S, or NR²;

X² is O, S, or NR²;

Y is O, S, or NR²;

n is 0, 1, 2, or 3; and

p is 0, 1, 2, or 3.

In certain embodiments, the invention relates to any one of the aforementioned compounds, provided the compound is not

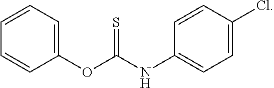 [SMILES: S=C(Nc1ccc(Cl)cc1)Oc1ccccc1]

In certain embodiments, the invention relates to any one of the aforementioned compounds, wherein

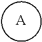

is optionally substituted aryl. In certain embodiments, the invention relates to any one of the aforementioned compounds, wherein

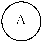

is optionally substituted phenyl or optionally substituted naphthyl. In certain embodiments, the invention relates to any one of the aforementioned compounds, wherein n is 1, 2, or 3; and

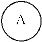

is para-substituted phenyl. In certain embodiments, the invention relates to any one of the aforementioned compounds, wherein n is 1, 2, or 3; and

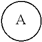

is ortho-substituted phenyl. In certain embodiments, the invention relates to any one of the aforementioned compounds, wherein n is 1; and

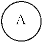

is para-substituted phenyl. In certain embodiments, the invention relates to any one of the aforementioned compounds, wherein n is 1; and

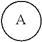

is meta-substituted phenyl. In certain embodiments, the invention relates to any one of the aforementioned compounds, wherein n is 1; and

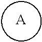

is ortho-substituted phenyl. In certain embodiments, the invention relates to any one of the aforementioned compounds, wherein

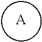

is naphthyl. In certain embodiments, the invention relates to any one of the aforementioned compounds, wherein

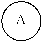

is 2-naphthyl.

In certain embodiments, the invention relates to any one of the aforementioned compounds, wherein

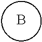

is optionally substituted aryl. In certain embodiments, the invention relates to any one of the aforementioned compounds, wherein

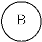

is optionally substituted phenyl. In certain embodiments, the invention relates to any one of the aforementioned compounds, wherein

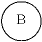

is phenyl; and

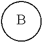

does not comprise any optional substituents. In certain embodiments, the invention relates to any one of the aforementioned compounds, wherein

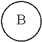

is para-substituted phenyl.

In certain embodiments, the invention relates to any one of the aforementioned compounds, wherein R¹ is iodo, bromo, chloro, or fluoro. In certain embodiments, the invention relates to any one of the aforementioned compounds, wherein n is 1; and R¹ is iodo, bromo, chloro, or fluoro.

In certain embodiments, the invention relates to any one of the aforementioned compounds, wherein R¹ is optionally substituted alkyl. In certain embodiments, the invention relates to any one of the aforementioned compounds, wherein R¹ is aminoalkyl. In certain embodiments, the invention relates to any one of the aforementioned compounds, wherein R¹ is protected aminoalkyl.

In certain embodiments, the invention relates to any one of the aforementioned compounds, wherein R² is —H or optionally substituted alkyl. In certain embodiments, the invention relates to any one of the aforementioned compounds, wherein R² is —H.

In certain embodiments, the invention relates to any one of the aforementioned compounds, wherein R³ is —H. In certain embodiments, the invention relates to any one of the aforementioned compounds, wherein R³ is optionally substituted aralkyl. In certain embodiments, the invention relates to any one of the aforementioned compounds, wherein R³ is optionally substituted benzyl. In certain embodiments, the invention relates to any one of the aforementioned compounds, wherein R³ is para-substituted benzyl. In certain embodiments, the invention relates to any one of the aforementioned compounds, wherein R³ is halo-substituted benzyl. In certain embodiments, the invention relates to any one of the aforementioned compounds, wherein R³ is chloro-substituted benzyl. In certain embodiments, the invention relates to any one of the aforementioned compounds, wherein R³ is 4-chlorobenzyl. In certain embodiments, the invention relates to any one of the aforementioned compounds, wherein R³ is —C(O)OR². In certain embodiments, the invention relates to any one of the aforementioned compounds, wherein R³ is —C(O)OR²; and R² is optionally substituted alkyl. In certain embodiments, the invention relates to any one of the aforementioned compounds, wherein R³ is —C(O)OR²; and R² is t-butyl.

In certain embodiments, the invention relates to any one of the aforementioned compounds, wherein X¹ is O or NR². In certain embodiments, the invention relates to any one of the aforementioned compounds, wherein X¹ is O.

In certain embodiments, the invention relates to any one of the aforementioned compounds, wherein X² is O or NR². In certain embodiments, the invention relates to any one of the aforementioned compounds, wherein X² is O. In certain embodiments, the invention relates to any one of the aforementioned compounds, wherein X² is NR². In certain embodiments, the invention relates to any one of the aforementioned compounds, wherein X² is NH.

In certain embodiments, the invention relates to any one of the aforementioned compounds, wherein Y is O. In certain embodiments, the invention relates to any one of the aforementioned compounds, wherein Y is S.

In certain embodiments, the invention relates to any one of the aforementioned compounds, wherein n is 0. In certain embodiments, the invention relates to any one of the aforementioned compounds, wherein n is 1.

In certain embodiments, the invention relates to any one of the aforementioned compounds, wherein m is 1.

In certain embodiments, the invention relates to any one of the aforementioned compounds, wherein the optional substituent, when present, is selected from the group consisting of alkoxy, alkyl ester, alkylcarbonyl, hydroxyalkyl, cyano, halo, amino, cycloalkyl, aryl, haloalkyl, nitro, hydroxy, alkoxy, aryloxy, alkyl, alkylthio, and cyanoalkyl.

In certain embodiments, the invention relates to any one of the aforementioned compounds, wherein the compound is a pharmaceutically acceptable salt.

In certain embodiments, the invention relates to any one of the aforementioned compounds, wherein the compound has a molecular weight less than about 300 Da.

In certain embodiments, the invention relates to a compound selected from the group consisting of:

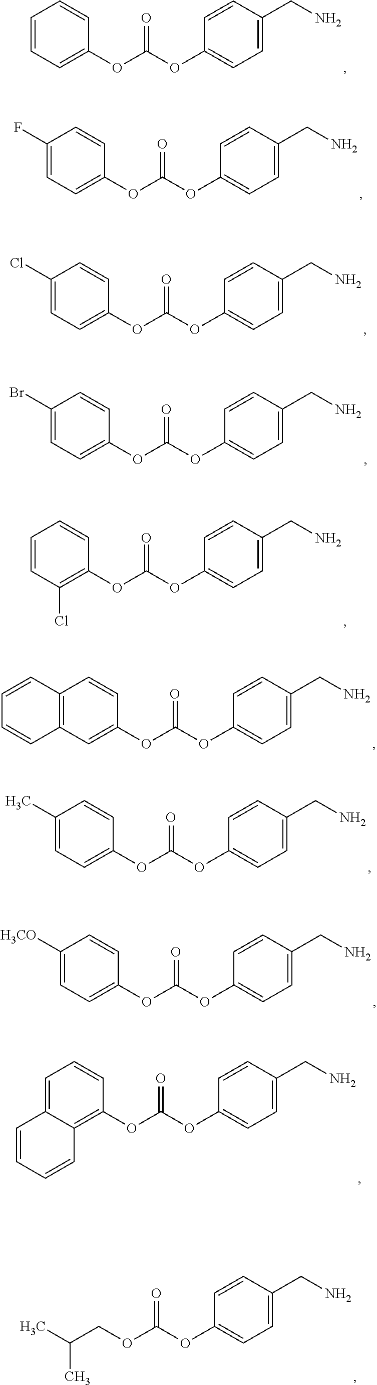

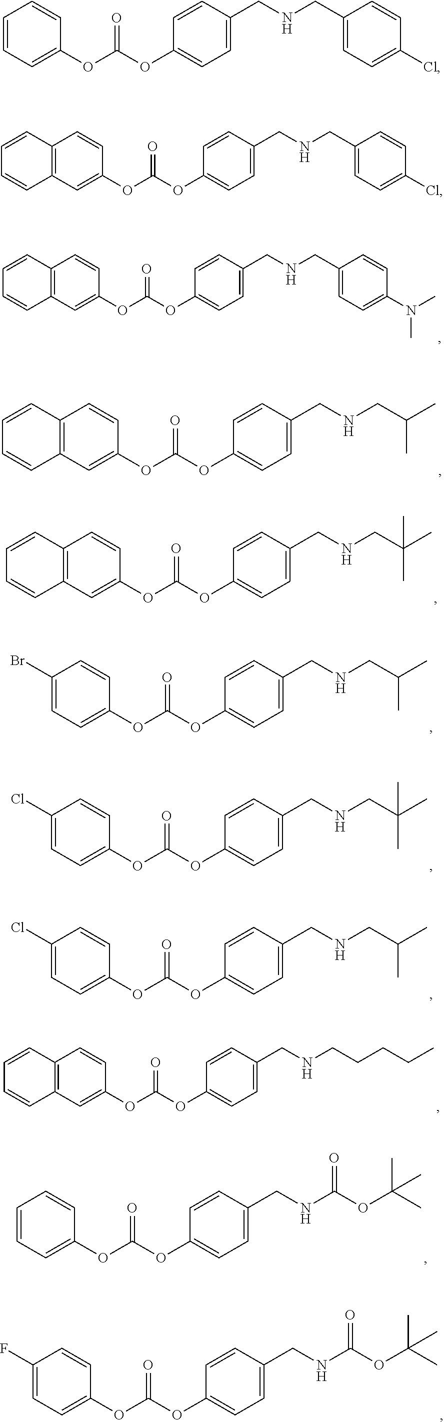

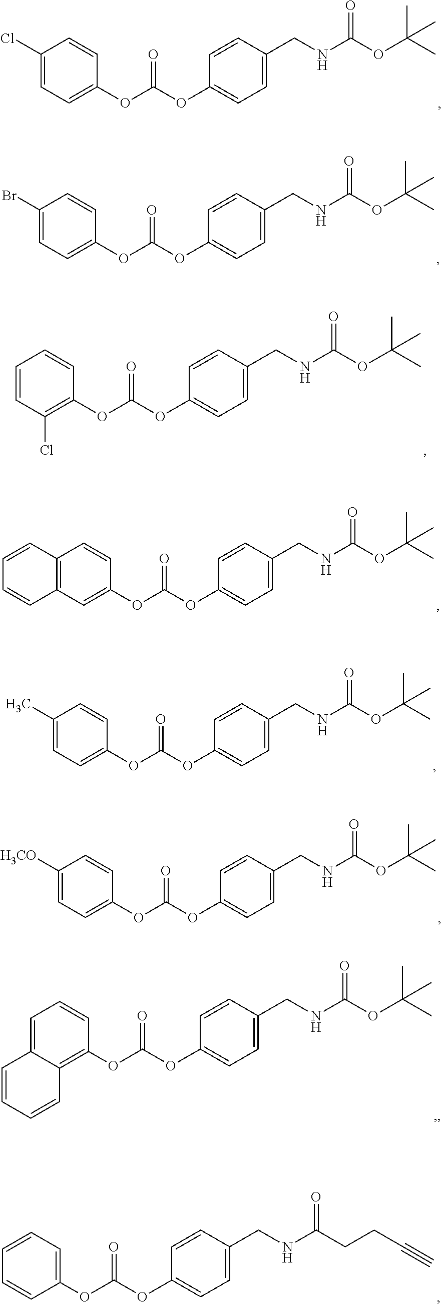

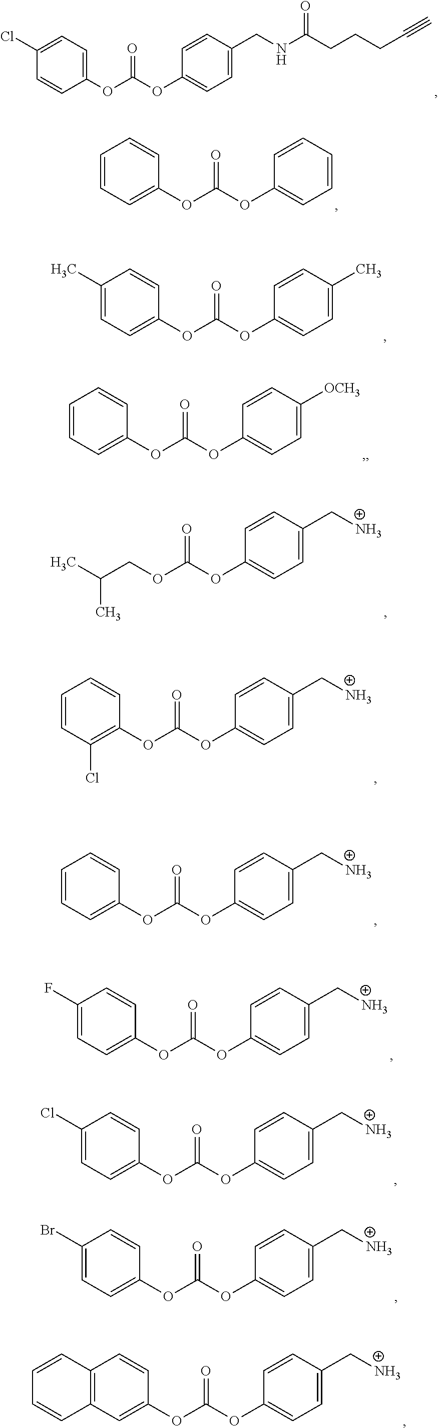

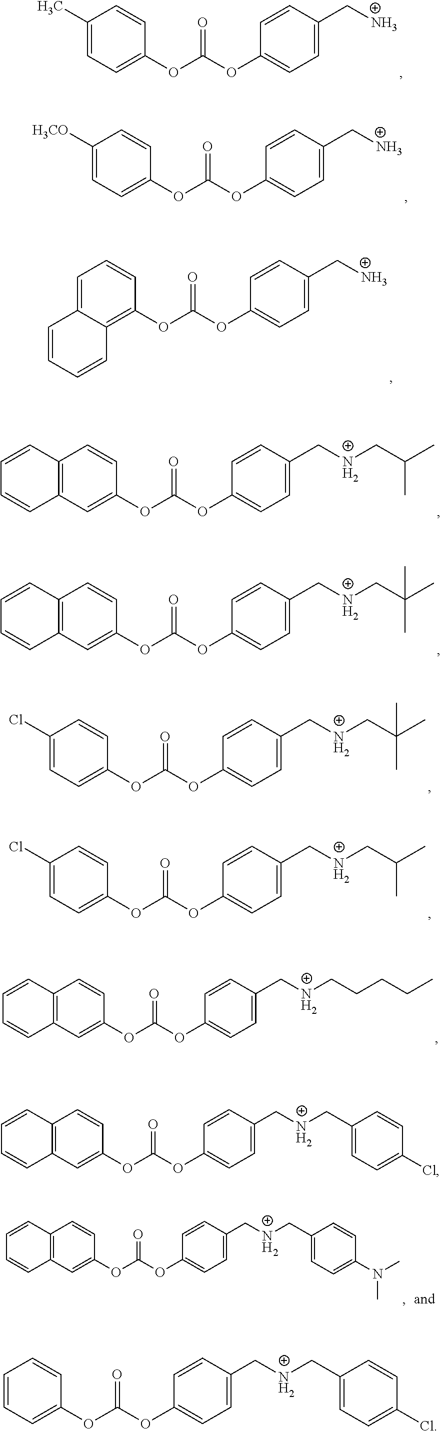

In certain embodiments, the invention relates to a compound selected from the group consisting of:

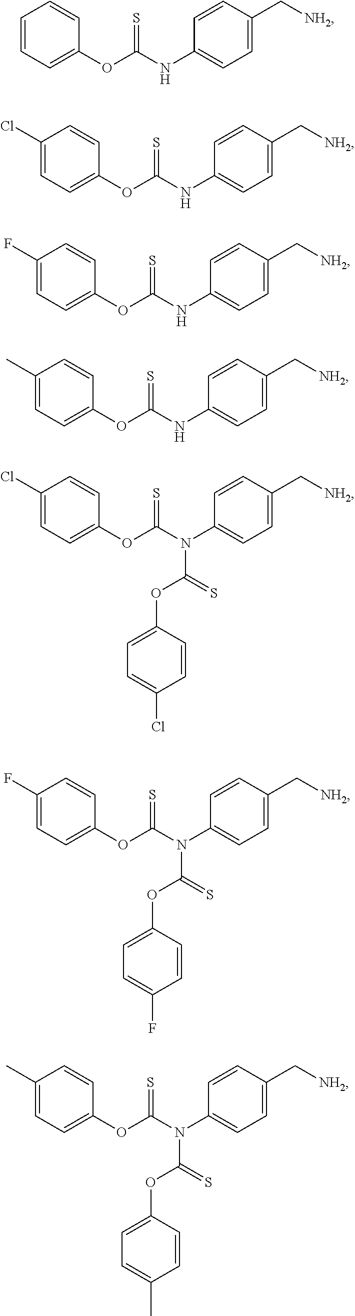

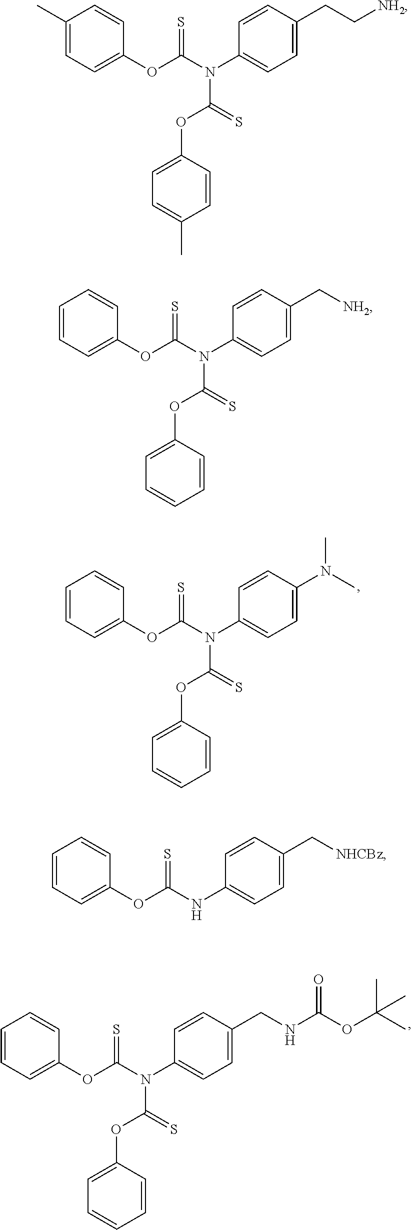

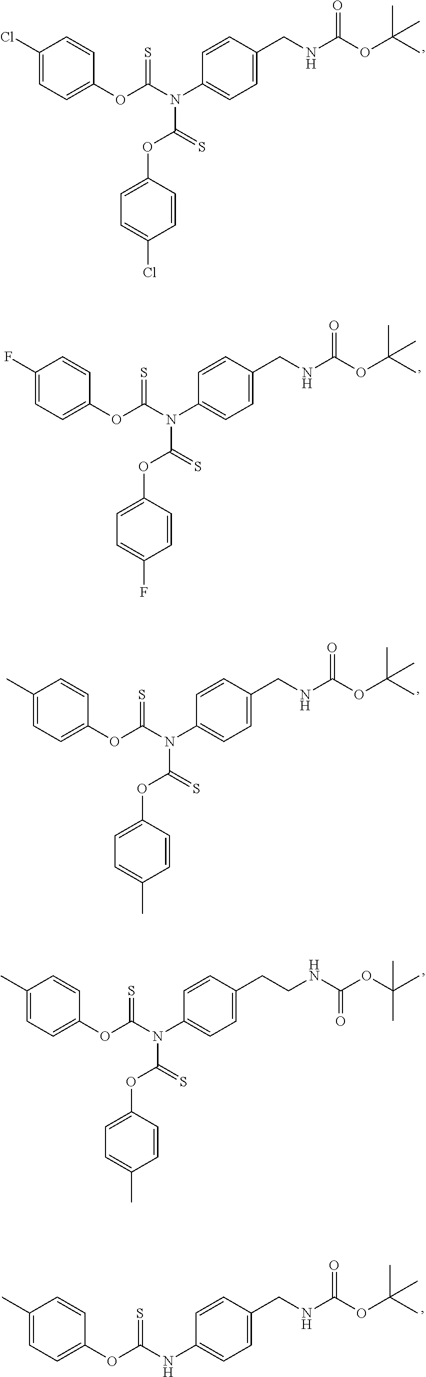

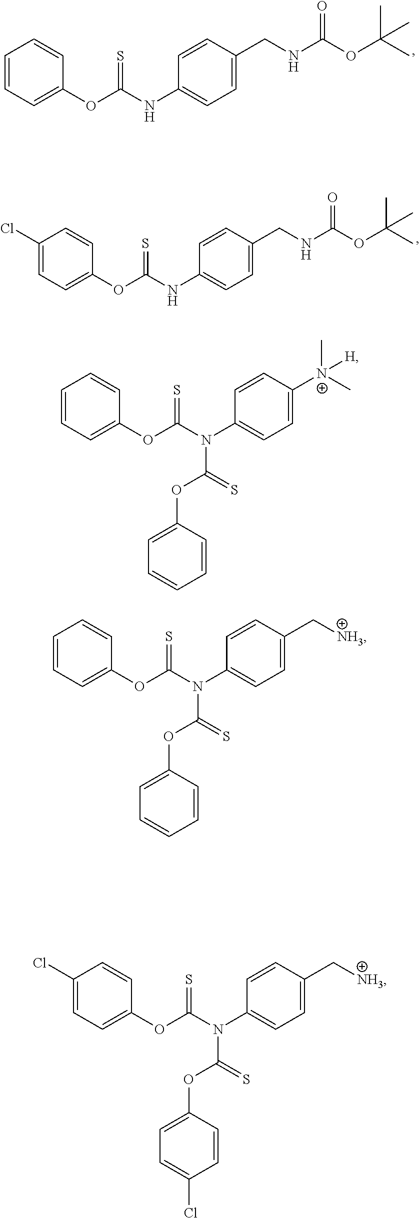

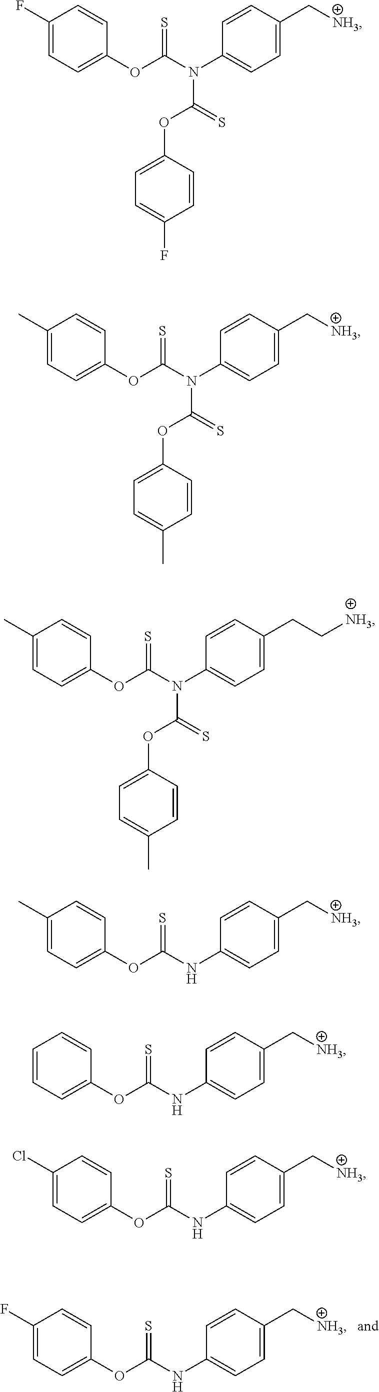

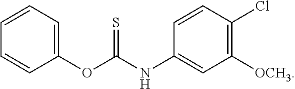 [SMILES: COc1cc(NC(=S)Oc2ccccc2)ccc1Cl]

In certain embodiments, the invention relates to a compound selected from the group consisting of:

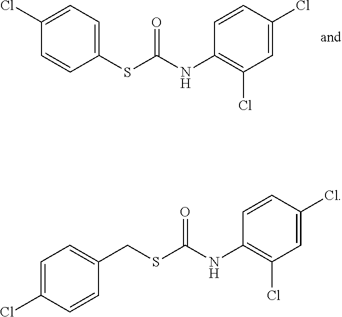 [SMILES: O=C(Nc1ccc(Cl)cc1Cl)Sc1ccc(Cl)cc1 ; O=C(Nc1ccc(Cl)cc1Cl)SCc1ccc(Cl)cc1]

In certain embodiments, the invention relates to a compound selected from the group consisting of:

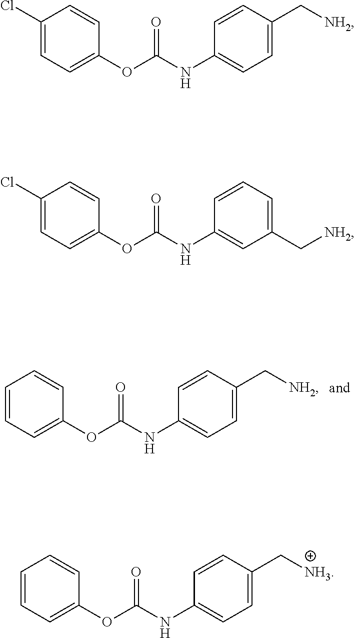

In certain embodiments, the invention relates to a compound selected from the group consisting of:

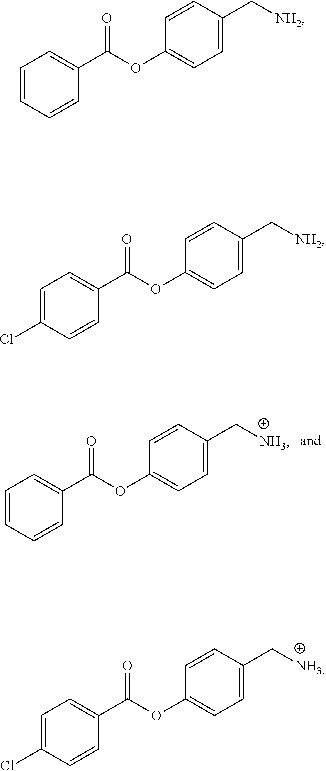

In certain embodiments, the invention relates to a compound selected from the group consisting of:

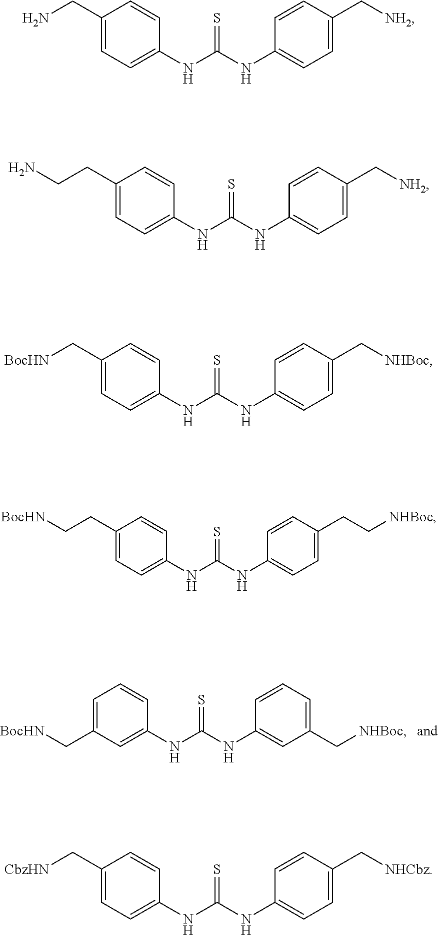 [SMILES: NCc1ccc(NC(=S)Nc2ccc(CN)cc2)cc1 ; NCCc1ccc(NC(=S)Nc2ccc(CN)cc2)cc1 ; CC(C)(C)OC(=O)NCc1ccc(NC(=S)Nc2ccc(CNC(=O)OC(C)(C)C)cc2)cc1 ; CC(C)(C)OC(=O)NCCc1ccc(NC(=S)Nc2ccc(CCNC(=O)OC(C)(C)C)cc2)cc1 ; CC(C)(C)OC(=O)NCc1cccc(NC(=S)Nc2cccc(CNC(=O)OC(C)(C)C)c2)c1 ; O=C(NCc1ccc(NC(=S)Nc2ccc(CNC(=O)OCc3ccccc3)cc2)cc1)OCc1ccccc1]

In certain embodiments, the invention relates to a compound of Formula III or Formula IV:

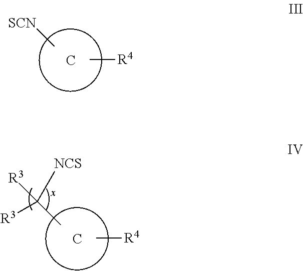

or a pharmaceutically acceptable salt thereof,
wherein, independently for each occurrence,

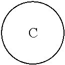

is aryl or heteroaryl;
x is 3, 4, or 5;
R³ is —H, optionally substituted alkyl, optionally substituted aryl, optionally substituted heteroaryl, optionally substituted aralkyl, optionally substituted heteroaralkyl, —C(O)R², or —C(O)OR²;
R² is —H, optionally substituted alkyl, optionally substituted aryl, or optionally substituted heteroaryl; and
R⁴ is absent, or is optionally substituted aminoalkyl, cyano, halo, optionally substituted alkyl, optionally substituted amino, or nitro.

In certain embodiments, the invention relates to any one of the aforementioned compounds, provided the compound is not

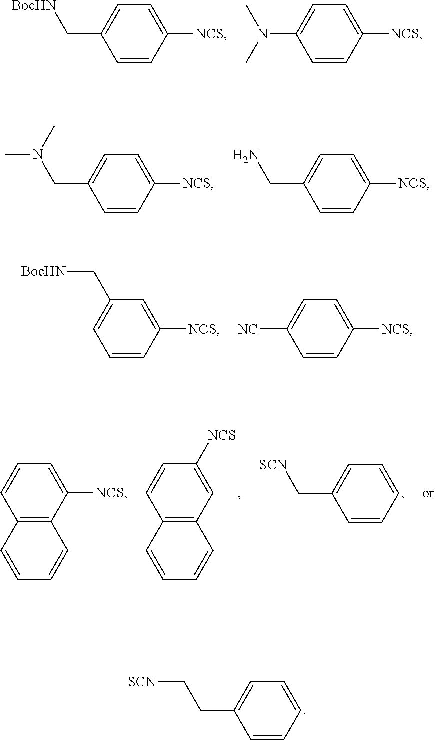

In certain embodiments, the invention relates to any one of the aforementioned compounds, wherein

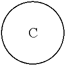

is aryl. In certain embodiments, the invention relates to any one of the aforementioned compounds, wherein

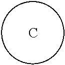

is phenyl or naphthyl. In certain embodiments, the invention relates to any one of the aforementioned compounds, wherein R⁴ is present; and

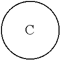

is para-substituted phenyl. In certain embodiments, the invention relates to any one of the aforementioned compounds, wherein R⁴ is present; and

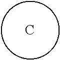

is meta-substituted phenyl.

In certain embodiments, the invention relates to any one of the aforementioned compounds, wherein R³ is —H.

In certain embodiments, the invention relates to any one of the aforementioned compounds, wherein R⁴ is absent. In certain embodiments, the invention relates to any one of the aforementioned compounds, wherein R⁴ is substituted aminoalkyl.

In certain embodiments, the invention relates to any one of the aforementioned compounds, wherein the compound is a pharmaceutically acceptable salt.

In certain embodiments, the invention relates to any one of the aforementioned compounds, wherein the compound has a molecular weight less than about 300 Da.

In certain embodiments, the invention relates to a compound selected from the group consisting of:

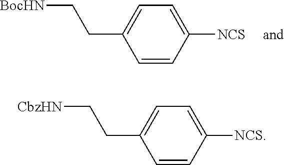

### Exemplary Pharmaceutical Compositions

In certain embodiments, the invention relates to a pharmaceutical composition comprising any one of the aforementioned compounds and a pharmaceutically acceptable carrier.

Patients, including but not limited to humans, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

In certain embodiments, a dose of the compound will be in the range of about 0.1 to about 100 mg/kg, more generally, about 1 to 50 mg/kg, and, preferably, about 1 to about 20 mg/kg, of body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3,000 mg, preferably 70 to 1400 mg of active ingredient per unit dosage form. An oral dosage of 50-1,000 mg is usually convenient.

Ideally the active ingredient should be administered to achieve peak plasma concentrations of the active compound from about 0.2 to 70 μM, preferably about 1.0 to 15 μM. This can be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### EXEMPLARY METHODS OF THE INVENTION

In certain embodiments, the invention relates to a method of preventing or treating a disease in a subject in need thereof comprising the step of: administering to the subject a therapeutically effective amount of any one of the aforementioned compounds.

In certain embodiments, the invention relates to a method of preventing or treating a disease in a subject in need thereof comprising the step of: administering to the subject a therapeutically effective amount of a compound selected from the group consisting of:

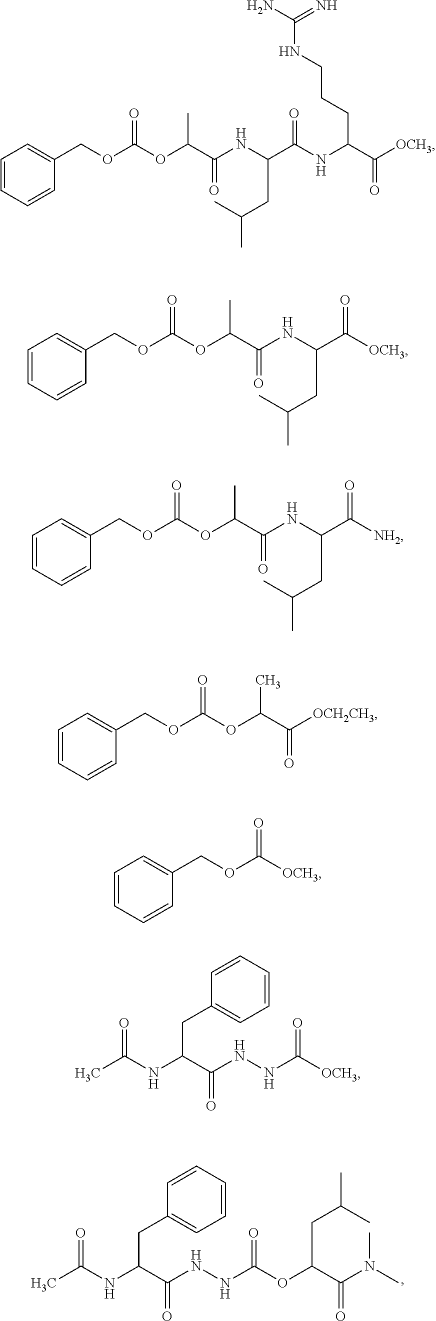

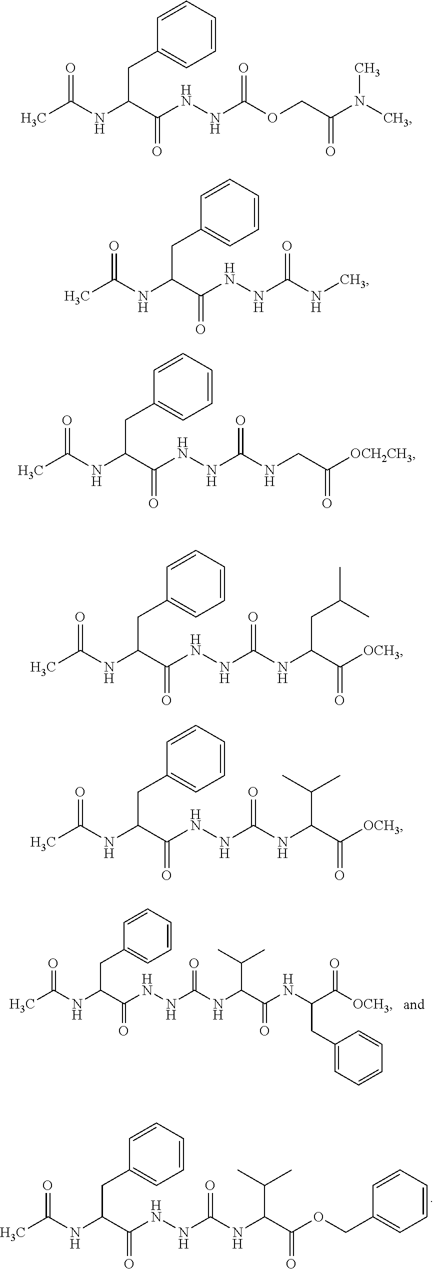

In certain embodiments, the invention relates to a method of preventing or treating a disease in a subject in need thereof comprising the step of: administering to the subject a therapeutically effective amount of a compound selected from the group consisting of:

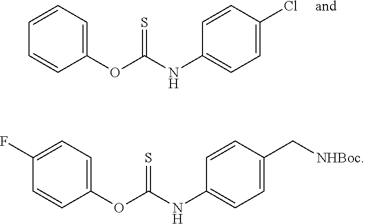 [SMILES: S=C(Nc1ccc(Cl)cc1)Oc1ccccc1 ; "and" ; CC(C)(C)OC(=O)NCc1ccc(NC(=S)Oc2ccc(F)cc2)cc1]

In certain embodiments, the invention relates to a method of preventing or treating a disease in a subject in need thereof comprising the step of: administering to the subject a therapeutically effective amount of a compound selected from the group consisting of:

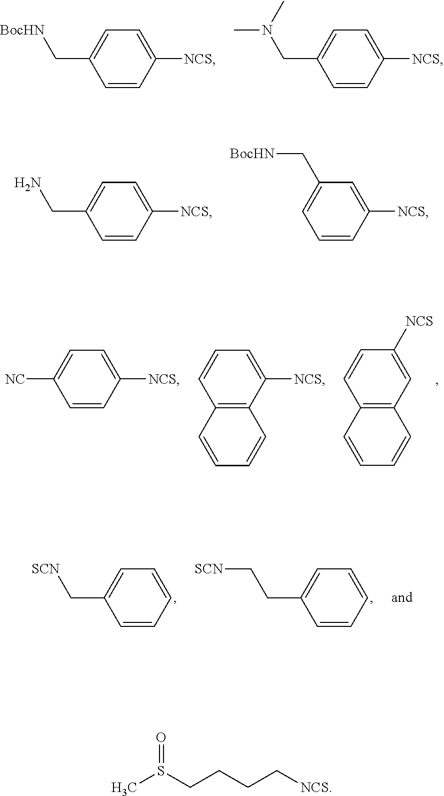

In certain embodiments, the invention relates to any one of the aforementioned methods, wherein the disease is a proteinopathy. Examples of such proteinopathies include, but are not limited to, Alzheimer's disease, cerebral β-amyloid angiopathy, retinal ganglion cell degeneration, prion diseases (e.g., bovine spongiform encephalopathy, kuru, Creutzfeldt-Jakob disease, variant Creutzfeldt-Jakob disease, Gerstmann-Straussler-Scheinker syndrome, fatal familial insomnia) tauopathies (e.g., frontotemporal dementia, Parkinson's disease, progressive supranuclear palsy, corticobasal degeration, frontotemporal lobar degeneration), frontemporal lobar degeneration, amyotrophic lateral sclerosis, Huntington's disease, familial British dementia, Familial Danish dementia, hereditary cerebral hemorrhage with amyloidosis (Icelandic), CADASIL, Alexander disease, Seipinopathies, familial amyloidotic neuropothy, senile systemic amyloidosis, serpinopathies, AL amyloidosis, AA amyloidosis, type II diabetes, aortic medial amyloidosis, ApoAI amyloidosis, ApoII amyloidosis, ApoAIV amyloidosis, familial amyloidosis of the Finish type, lysozyme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amylois, seminal vesical amyloid, cystric fibrosis, sickle cell disease, critical illness myopathy, von Hippel-Lindau disease, spinocerebellar ataxia 1, Angelman syndrome, giant axon neuropathy, inclusion body myopathy with Paget disease of bone, and frontotemporal dementia (IBMPFD).

In certain embodiments, the invention relates to any one of the aforementioned methods, wherein the disease is a cell proliferative disorder or disease. In certain embodiments, the disease is cancer, tumor, neoplasm, neovascularization, vascularization, cardiovascular disease, intravasation, extravasation, metastasis, arthritis, infection, blood clot, atherosclerosis, melanoma, skin disorder, rheumatoid arthritis, diabetic retinopathy, macular edema, or macular degeneration, inflammatory and arthritic disease, autoimmune disease or osteosarcoma. Certain therapeutic methods of the invention include treating malignancies, including solid tumors and disseminated cancers. Exemplary tumors that may be treated in accordance with the invention include e.g., cancers of the lung, prostate, breast, liver, colon, breast, kidney, pancreas, brain, skin including malignant melanoma and Kaposi's sarcoma, testes or ovaries, or leukemias or lymphoma including Hodgkin's disease. Exemplary autoimmune diseases include, but are not limited to lupus.

In certain embodiments, the invention relates to any one of the aforementioned methods, wherein the disease is an infection.

In certain embodiments, the invention relates to any one of the aforementioned methods, wherein the infection is a protozoan, helminthic, fungal, bacterial, or viral infection.

In certain embodiments, the invention relates to any one of the aforementioned methods, wherein the infection is malaria, toxoplasmosis, schistosomaisis, a trypanosomal parasitic infection, Chagas' disease, leishmaniasis, or human African trypanosomiasis.

In certain embodiments, the invention relates to any one of the aforementioned methods, wherein the infection is an Entamoeba histolytica infection or a Giardia infection.

In certain embodiments, the invention relates to any one of the aforementioned methods, wherein the infection is an Opisthorchis viverrini infection, a Clonorchis sinensis infection, an Angiostrongylus cantonensis infection, an Angiostrongylus cantonensis infection, a Fasciola hepatica infection, a Fasciola gigantica infection, a Dictyocaulus viviparous infection, a Haemonchus contortus infection, or a Schistosoma infection.

In certain embodiments, the invention relates to any one of the aforementioned methods, wherein the infection is a Cryptococcus neoformans infection.

In certain embodiments, the invention relates to any one of the aforementioned methods, wherein the infection is a SARS infection, a Picornaviral infection, a Coronaviral infection, a Epstein Barr infection, an arterivirus or a nairovirus infection, a Kaposi's sarcoma-associated herpesvirus infection, a foot-and-mouth disease virus infection, a Crimean Congo hemorrhagic fever virus (CCHFV) infection, a Hepatitis B virus infection, or a human cytomegalovirus infection.

In certain embodiments, the invention relates to any one of the aforementioned methods, wherein the infection is a Staphylococcus aureus infection, Porphyromonas gingivalis infection, a Yersinia pestis infection, a Salmonella infection, a Chlamydia infection, or a Clostridium difficile infection.

In certain embodiments, the invention relates to any one of the aforementioned methods, wherein the subject is a mammal. In certain embodiments, the invention relates to any one of the aforementioned methods, wherein the subject is human.

In certain embodiments, the invention relates to a method of inhibiting a cysteine protease comprising the step of: contacting the cysteine protease with an effective amount of any one of the aforementioned compounds.

In certain embodiments, the invention relates to a method of inhibiting a cysteine protease comprising the step of: contacting the cysteine protease with an effective amount of a compound selected from the group consisting of:

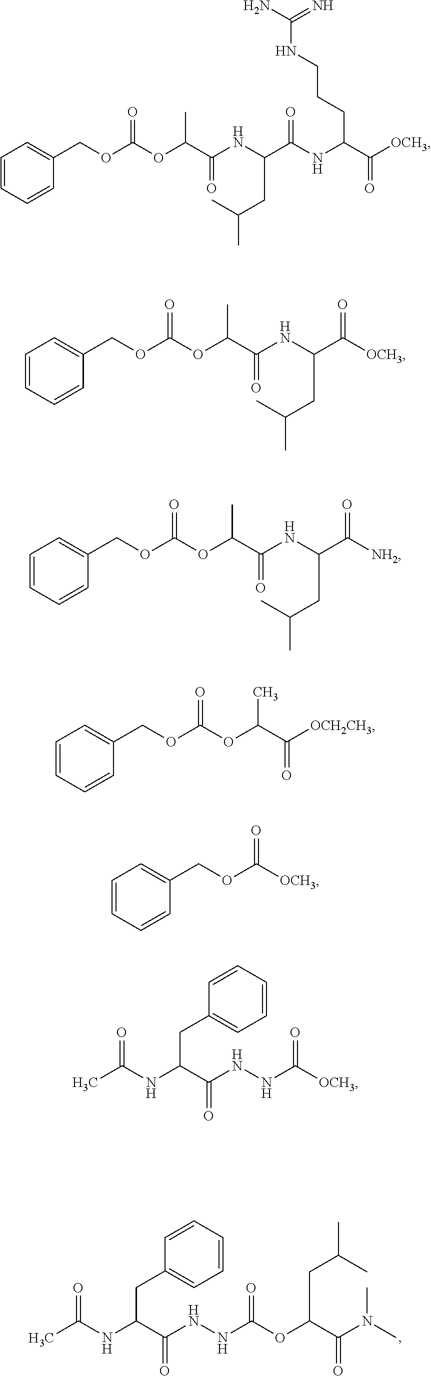

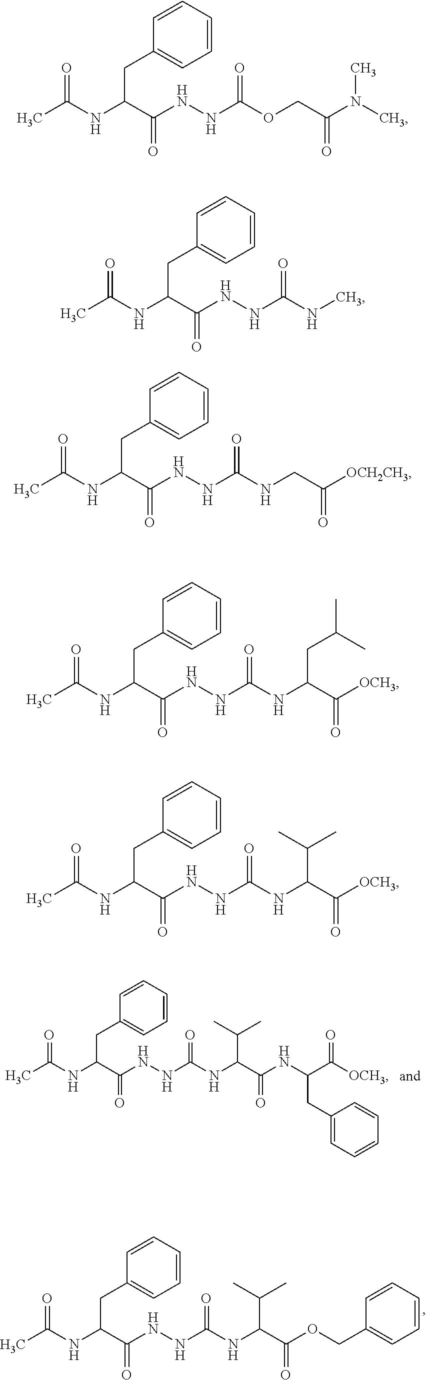

wherein the cysteine protease is not papain.

In certain embodiments, the invention relates to a method of inhibiting a cysteine protease comprising the step of: contacting the cysteine protease with an effective amount of a compound selected from the group consisting of:

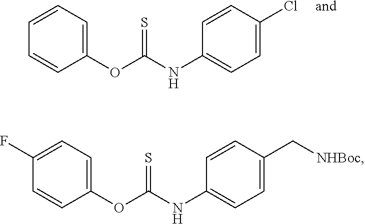 [SMILES: S=C(Nc1ccc(Cl)cc1)Oc1ccccc1 ; "and" ; CC(C)(C)OC(=O)NCc1ccc(NC(=S)Oc2ccc(F)cc2)cc1]

wherein the cysteine protease is not papain.

In certain embodiments, the invention relates to a method of inhibiting a cysteine protease comprising the step of: contacting the cysteine protease with an effective amount of a compound selected from the group consisting of:

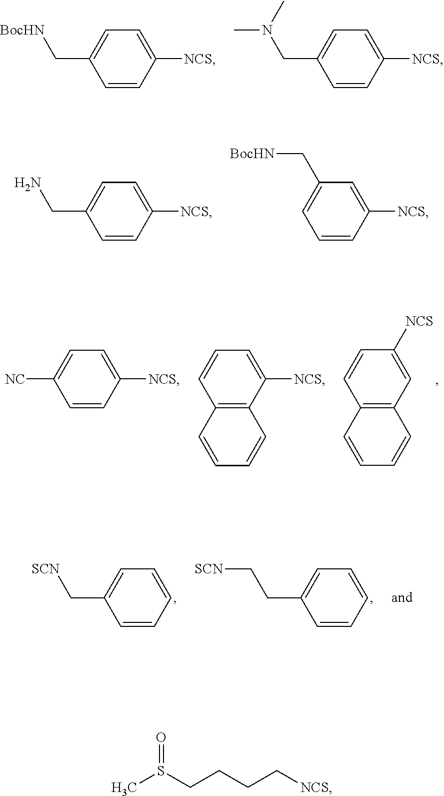

wherein the cysteine protease is not papain.

In certain embodiments, the invention relates to any one of the aforementioned methods, wherein the cysteine protease is cathepsin. In certain embodiments, the invention relates to any one of the aforementioned methods, wherein the cysteine protease is cathepsin C. In certain embodiments, the invention relates to any one of the aforementioned methods, wherein the cysteine protease is cathepsin B. In certain embodiments, the invention relates to any one of the aforementioned methods, wherein the cysteine protease is cathepsin K. In certain embodiments, the invention relates to any one of the aforementioned methods, wherein the cysteine protease is cathepsin L. In general, cathepsins are involved in inflammatory or autoimmune diseases such as atherosclerosis, obesity, rheumatoid arthritis, cardiac repair, cardiomyopathy, and cancer.

In certain embodiments, the invention relates to any one of the aforementioned methods, wherein the cysteine protease is a MALT1 protease.

In certain embodiments, the invention relates to any one of the aforementioned methods, wherein the cysteine protease is a caspase or a calpain. Caspases are involved in cancer, inflammation, and neurodegeneration. Calpains are involved in necrosis, ischemia and reperfusion injury, neurological disorders, muscular dystrophies, cataract, cancer, diabetes, gastropathy, Alzheimer's disease, Parkinson's disease, atherosclerosis, and pulmonary hypertension.

In certain embodiments, the invention relates to any one of the aforementioned methods, wherein the cysteine protease is falcipain, cruzain, Leishmania CPA protease, Leishmania CPB protease, Leishmania CPS protease, an Entamoeba histolytica cysteine protease (e.g., EhCP1, EhCP2, or EhCP3), or a Giardia cysteine protease.

In certain embodiments, the invention relates to any one of the aforementioned methods, wherein the cysteine protease is an Opisthorchis viverrini cysteine protease, a Clonorchis sinensis cysteine protease, an Angiostrongylus cantonensis cathepsin B-like enzyme gene 1, 2 (e.g., AC-cathB-1, AC-cathB-2), an Angiostrongylus cantonensis hemoglobin-type cysteine protease, a Fasciola hepatica virulence-associated cysteine peptidase, a Fasciola gigantica protein, a bovine lungworm Dictyocaulus viviparous cysteine protease, a Haemonchus contortus cysteine protease, or a Schistosoma cysteine protease.

In certain embodiments, the invention relates to any one of the aforementioned methods, wherein the cysteine protease is Cryptococcus neoformans Ubp5.

In certain embodiments, the invention relates to any one of the aforementioned methods, wherein the cysteine protease is a SARS PL protease, a Picornaviral 3C protease, a Coronaviral 3C-like protease, a Epstein Barr virus deubiquitinating protease, an arterivirus or a nairovirus ovarian tumor domain-containing deubiquitinase, a Kaposi's sarcoma-associated herpesvirus-encoded deubiquitinase (e.g., ORF64), a foot-and-mouth disease virus (FMDV) papain-like proteinase, a Crimean Congo hemorrhagic fever virus (CCHFV) deubiquitinase, a Hepatitis B virus protein X, or a human cytomegalovirus high-molecular-weight protein (e.g., HMWP or pUL48)

In certain embodiments, the invention relates to any one of the aforementioned methods, wherein the cysteine protease is a Sortase transpeptidase from a Gram positive bacterium (e.g., Staphylococcus aureus), gingipain (e.g., from Porphyromonas gingivalis), a Yersinia pestis virulence factor (e.g., YopJ), an ElaD ortholog (e.g., Salmonella sseL), Chlamydia DUB1 or DUB2, Streptococcus pyogenes SpeB, Clostridium difficile Cwp84 or Cwp13 cysteine protease, toxin TcdA, or toxin TcdB.

In certain embodiments, the invention relates to any one of the aforementioned methods, wherein the cysteine protease is a deSUMOylase, a deNEDDylase, or a delSGylase.

In certain embodiments, the invention relates to any one of the aforementioned methods, wherein the compound is selective for the cysteine protease.

In certain embodiments, the invention relates to any one of the aforementioned methods, wherein the compound is specific for the cysteine protease.

In certain embodiments, the invention relates to any one of the aforementioned methods, wherein the cysteine protease is in vitro or in vivo.

In certain embodiments, the invention relates to any one of the aforementioned methods, wherein the compound is substantially cell permeable.

In certain embodiments, the invention relates to a method of inhibiting a deubiquitinating enzyme comprising the step of: contacting the deubiquitinating enzyme with an effective amount of any one of the aforementioned compounds.

In certain embodiments, the invention relates to a method of inhibiting a deubiquitinating enzyme comprising the step of: contacting the deubiquitinating enzyme with an effective amount of a compound selected from the group consisting of:

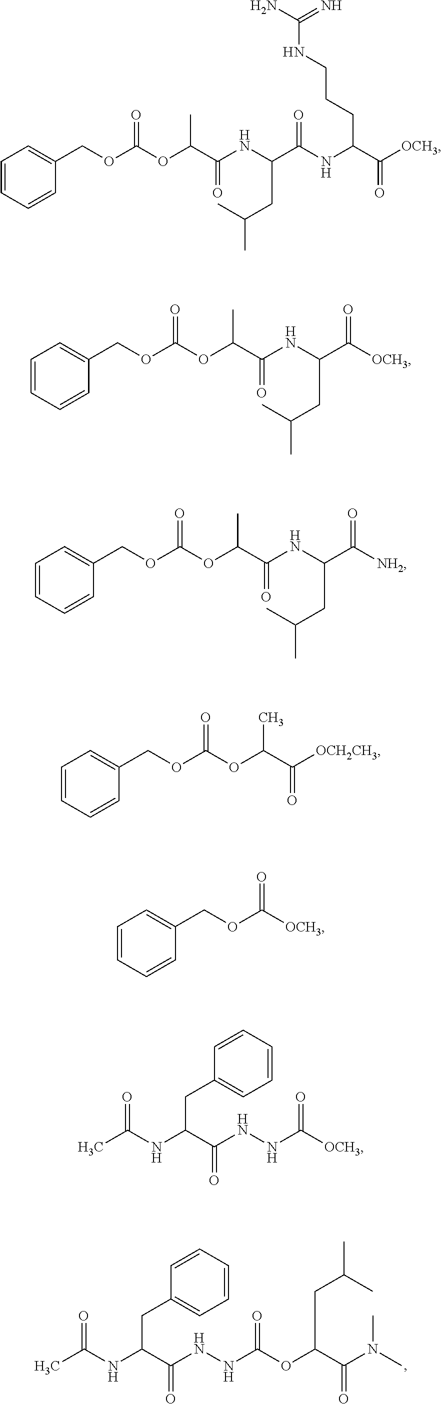

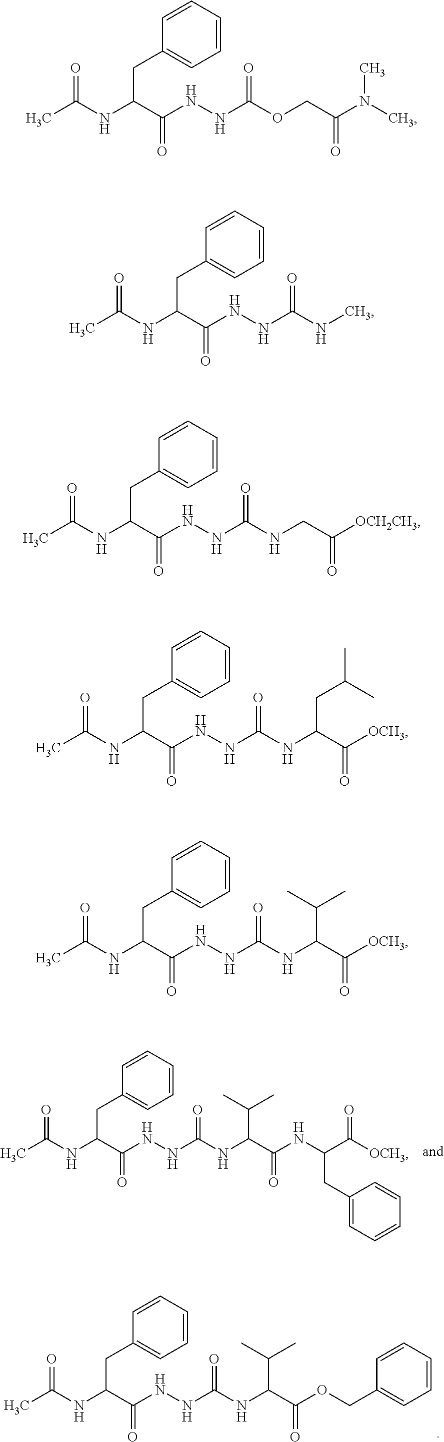

In certain embodiments, the invention relates to a method of inhibiting a deubiquitinating enzyme comprising the step of: contacting the deubiquitinating enzyme with an effective amount of a compound selected from the group consisting of:

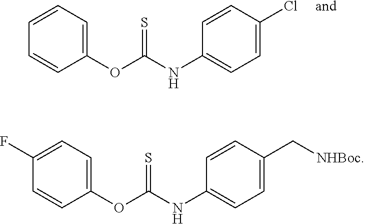 [SMILES: S=C(Nc1ccc(Cl)cc1)Oc1ccccc1 ; "and" ; CC(C)(C)OC(=O)NCc1ccc(NC(=S)Oc2ccc(F)cc2)cc1]

In certain embodiments, the invention relates to a method of inhibiting a deubiquitinating enzyme comprising the step of: contacting the deubiquitinating enzyme with an effective amount of a compound selected from the group consisting of:

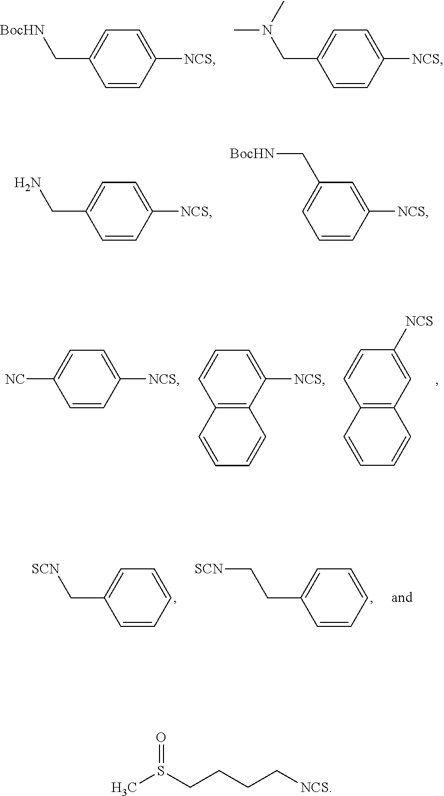

In certain embodiments, the invention relates to any one of the aforementioned methods, wherein the compound is selective for the deubiquitinating enzyme.

In certain embodiments, the invention relates to any one of the aforementioned methods, wherein the compound is specific for the deubiquitinating enzyme.

In certain embodiments, the invention relates to any one of the aforementioned methods, wherein the deubiquitinating enzyme is a member of the ubiquitin-specific processing protease (USP/UBP) superfamily or a member of the ubiquitin C-terminal hydrolyase (UCH) superfamily. In certain embodiments, the invention relates to any one of the aforementioned methods, wherein the deubiquitinating enzyme is selected from the group consisting of: USP9x, USP5, USP7, USP14, UCH37, and UCHL3.

In certain embodiments, the invention relates to any one of the aforementioned methods, wherein the deubiquitinating enzyme is in vitro or in vivo.

In certain embodiments, the invention relates to any one of the aforementioned methods, wherein the compound is substantially cell permeable.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Synthesis

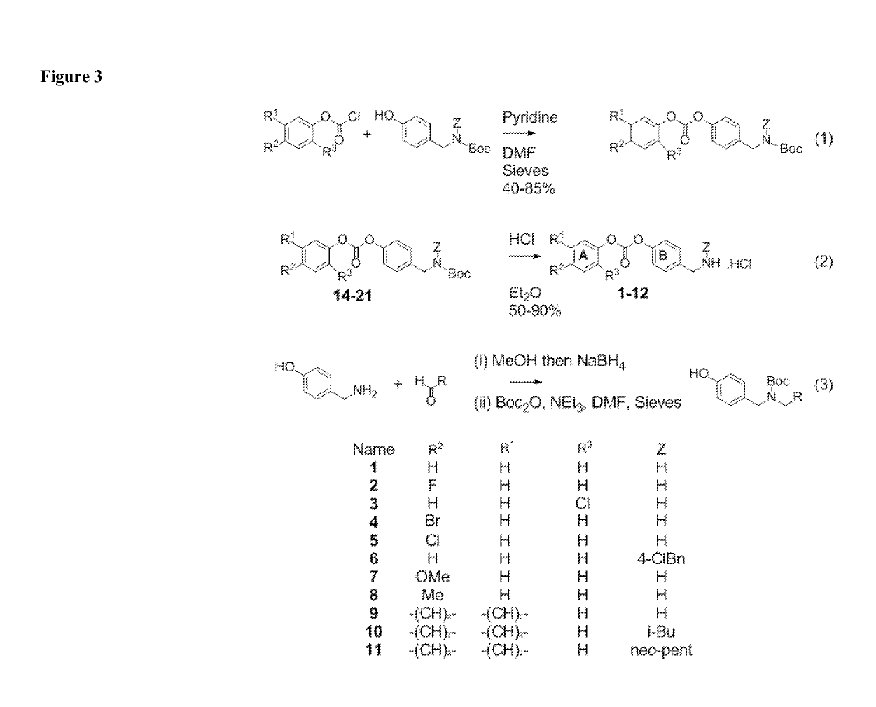

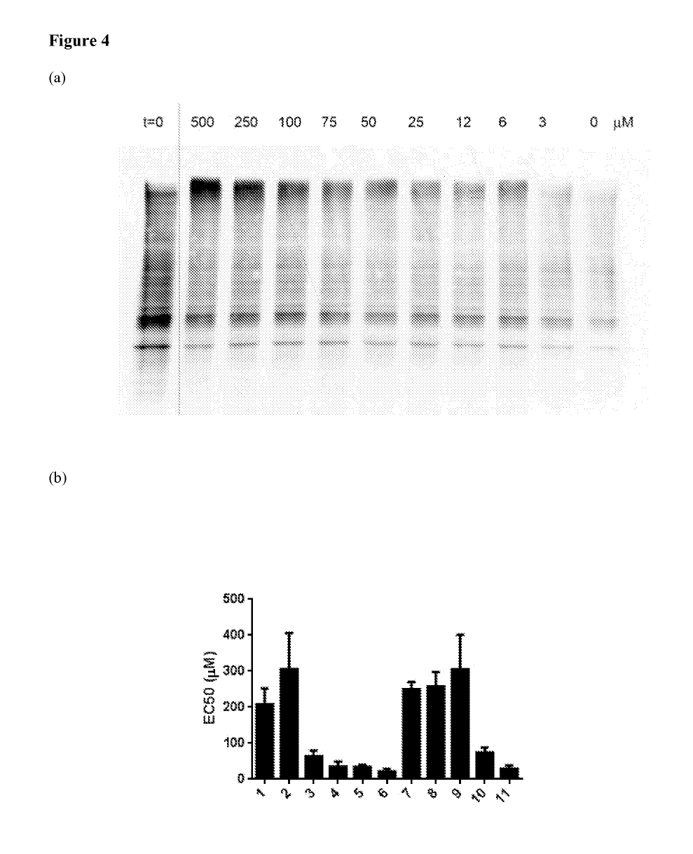

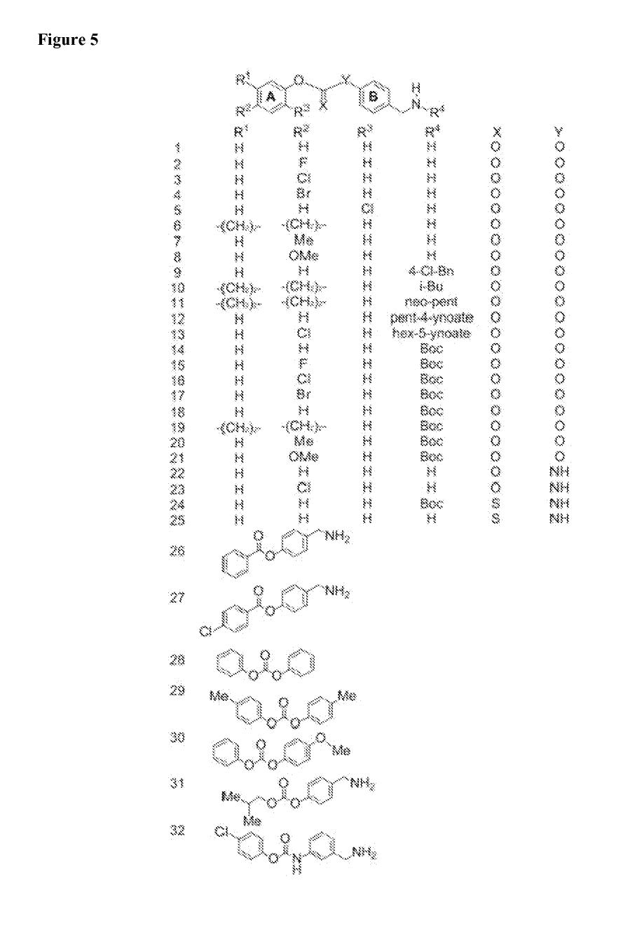

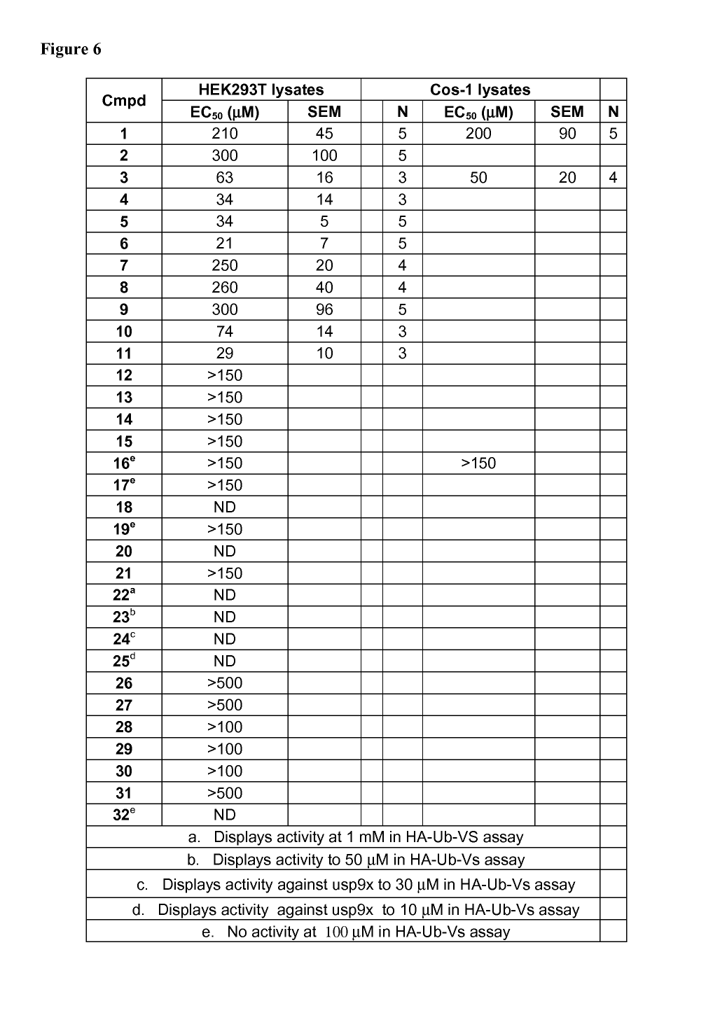

Most inhibitors were synthesized in two steps from commercially available starting materials. One chromatography step was required. See FIG. 3.

### Example 2

### A-Ring Substitution SAR

HEK293T lysates overexpressing ubiquiting-HA were treated with the stated compound for 1.5 h and the total ubiquitin pool was analyzed by western blot (HA). The SAR showed that a good leaving group is needed in the A ring (defined in FIG. 3). Substitution on the amino group is tolerated so long as a positive charge is maintained (16 is not an efficient inhibitor where 9, 10, and 11 (as defined in FIG. 3) have some potency). Similar data were obtained for Cos-1 lysates. See FIG. 4.

### Example 3

### Inhibition of USP9x and USP7

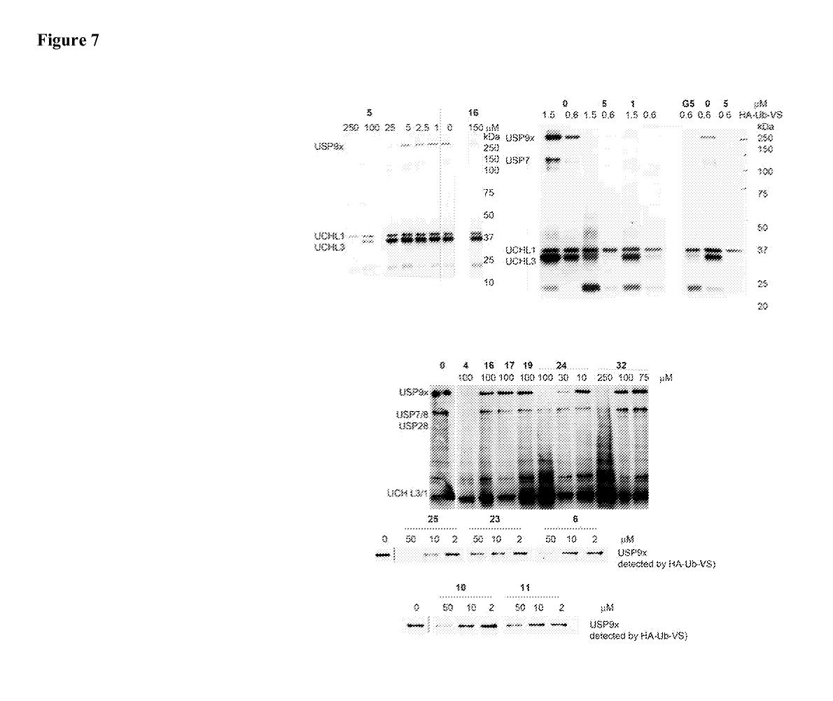

HA-ubiquitin vinylsulfone (HA-Ub-VS) irreversibly labels DUBs by modifying the catalytic cysteine residue Inhibition of the DUB prevents HA-Ub-VS labeling and the band is. Treatment of a HEK293T or Cos-1 lysate with 4 or 5 (defined in FIG. 3) prevents binding of HA-Ub-VS to USP9x (290 kDa) and USP7 (150 kDa) selectively. At higher concentrations UCHL1/3 (37 kDa) are inhibited. The interaction with UCHL1/3 is reversible. See FIG. 7.

### Example 4

### Cell Permeability of Inhibitors

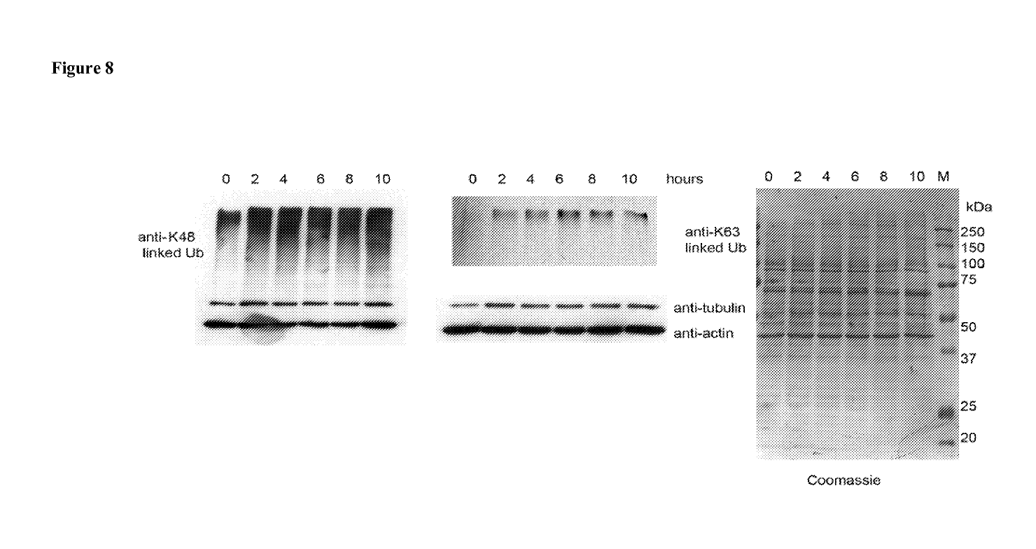

MCF7 cells treated with 4 (100 μM; defined in FIG. 3) show elevated K48- and K63-linked ubiquitin and increased molecular weight of chains. Importantly, the total proteome does not shift to higher molecular weights, as is observed when cells are treated with crosslinking agents such as G5. Similar results were obtained in Cos-1, CHO and HEK293T. See FIG. 8.

### Example 5

### Effect of Inhibitors on Proteasome or Caspases

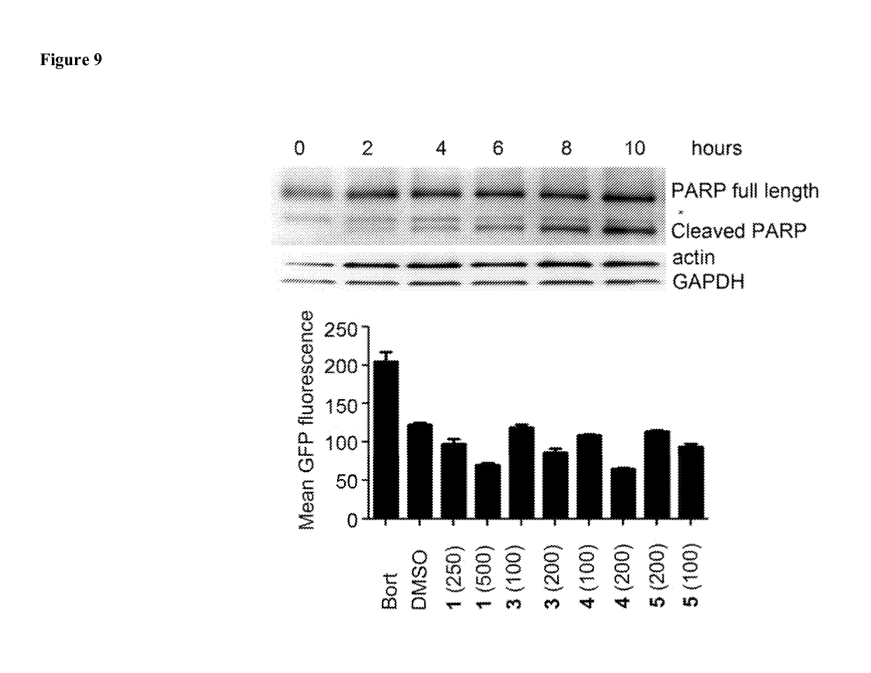

Inhibitor 4 (as defined in FIG. 3) does not inhibit caspases or the proteasome. Cells treated with 4 (100 μM; as defined in FIG. 3) show PARP cleavage that indicates caspases are active. See FIG. 9, top panel. The fluorescence of G76V ubiquitin-GFP fusion protein increases in the presence of inhibitor 4 (as defined in FIG. 3), as expected if degradation increases due to inhibition of DUBs. In contrast, GFP fluorescence decreases when the proteasome is inhibited by bortezomib. See FIG. 9, bottom panel.

### Example 6

### Cellular Response to DUB Inhibition

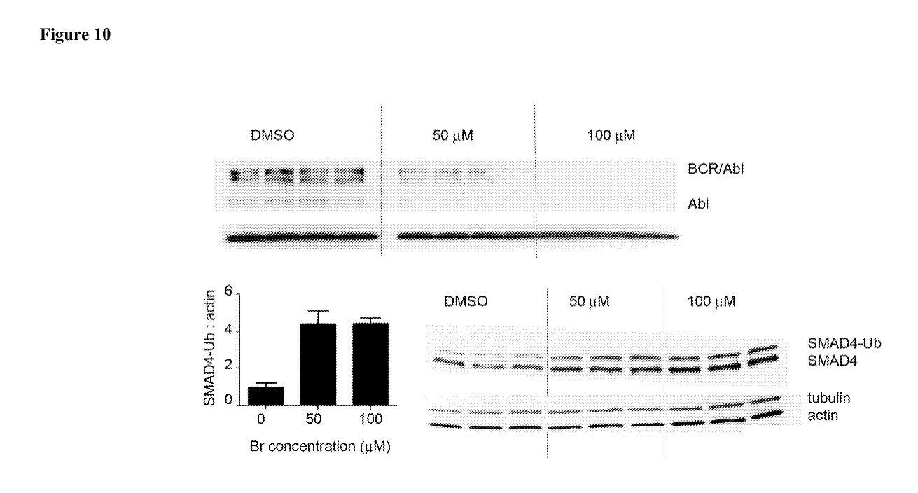

K562 cells treated with 4 (as defined in FIG. 3) show characteristics of USP9x knockout cell lines: decrease in BCR/Abl and increase in SMAD4 monoubiquitination (SMAD4-Ub). See FIG. 10.

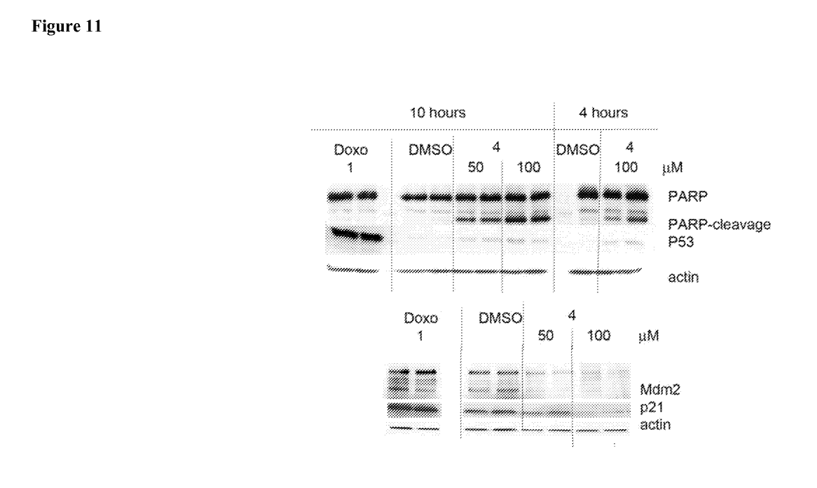

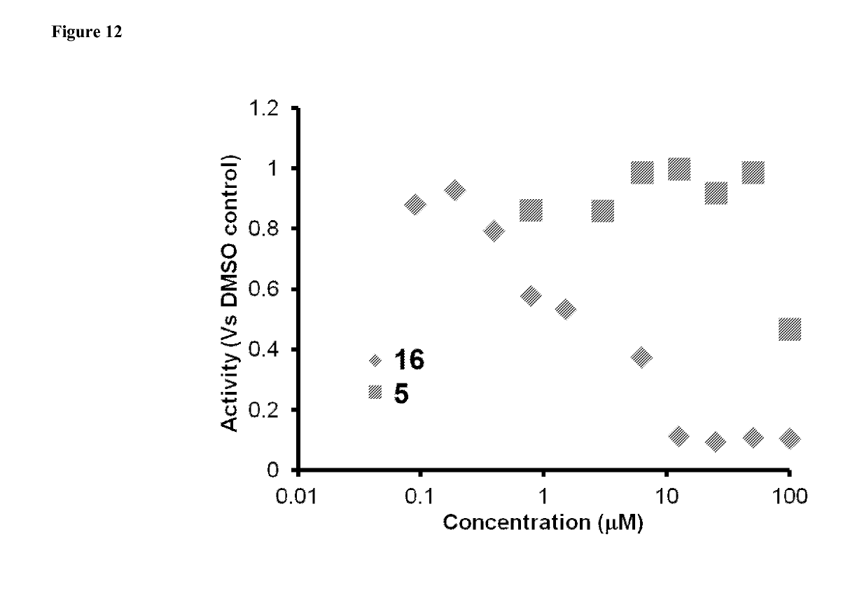

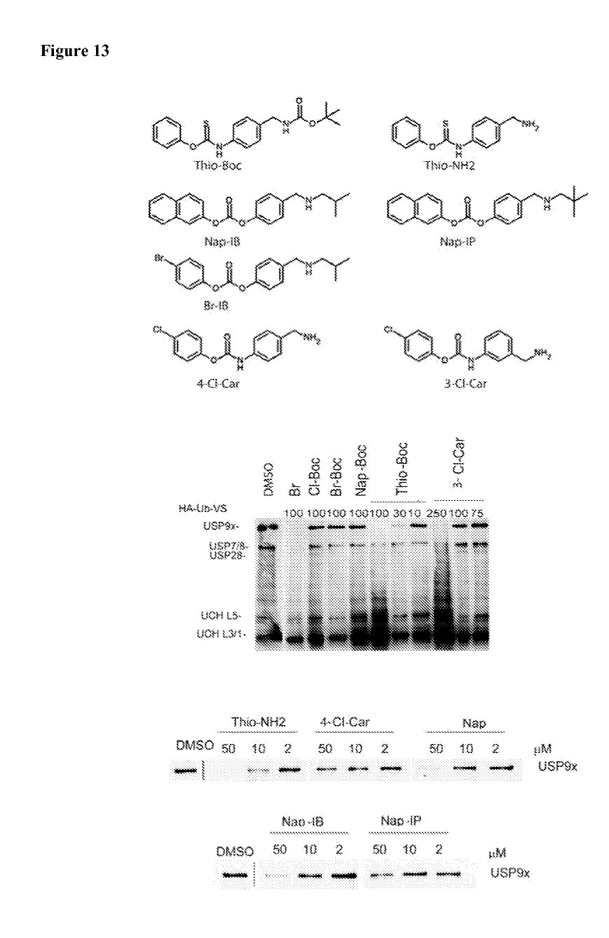

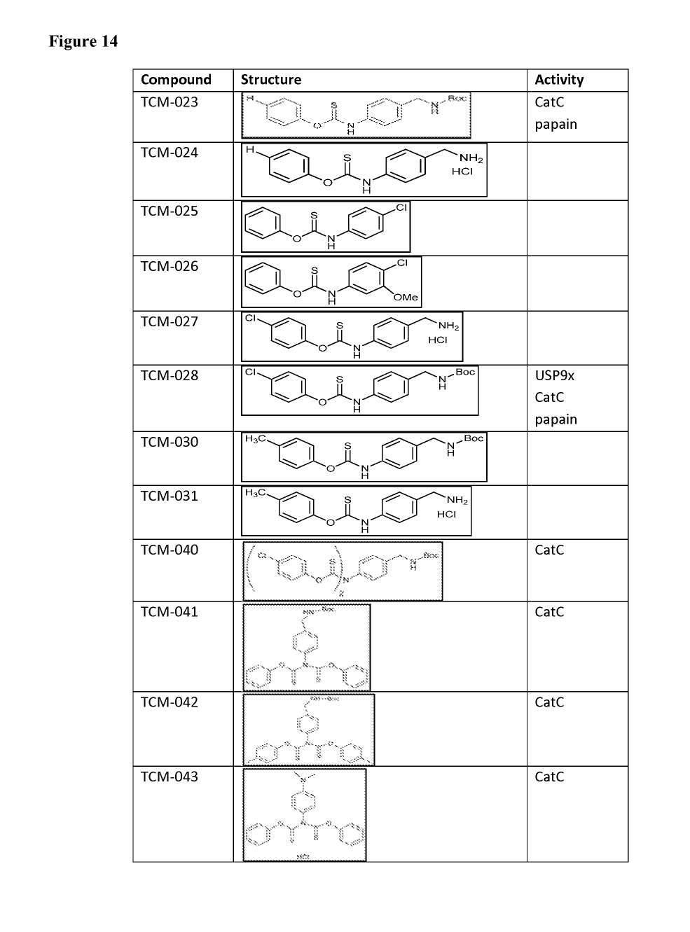

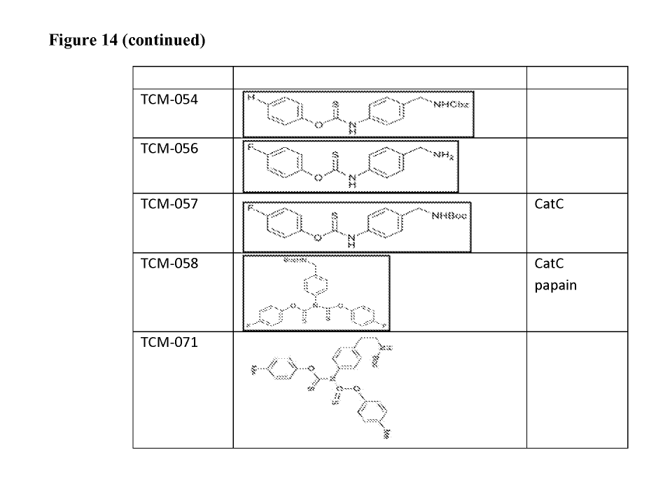

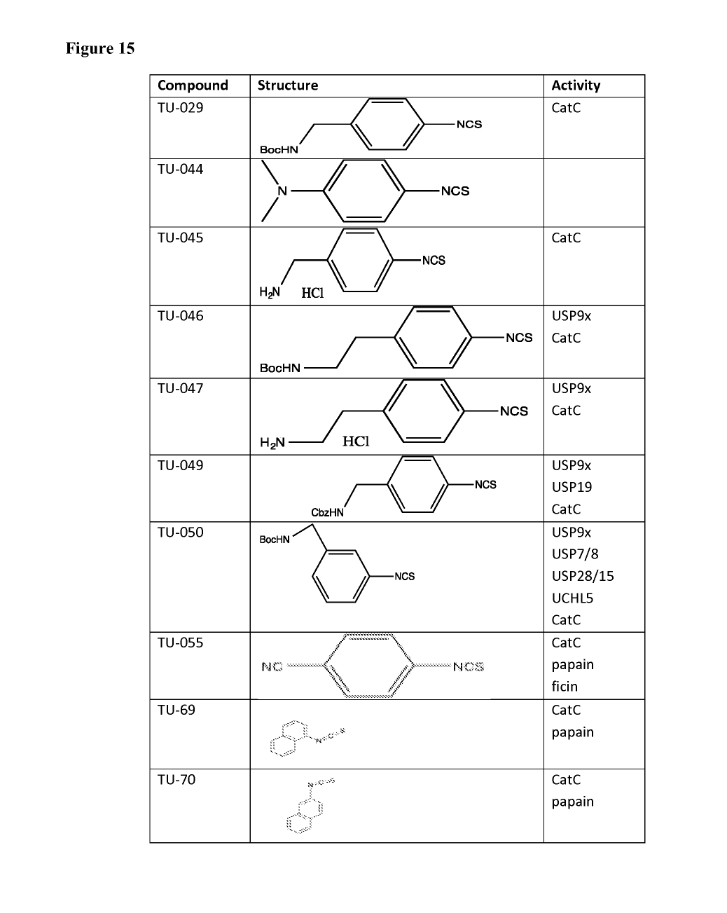

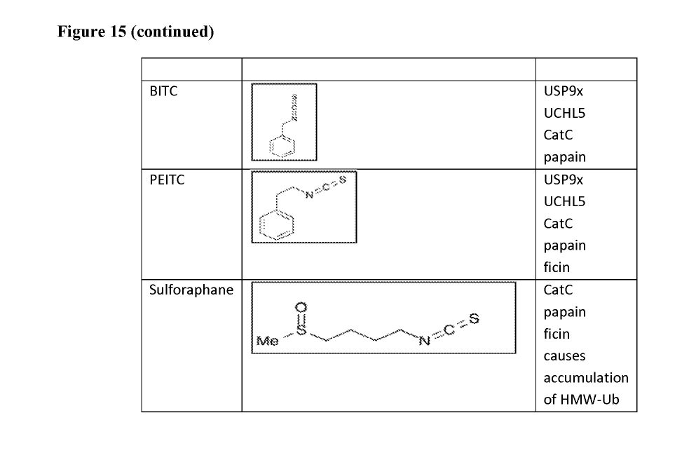

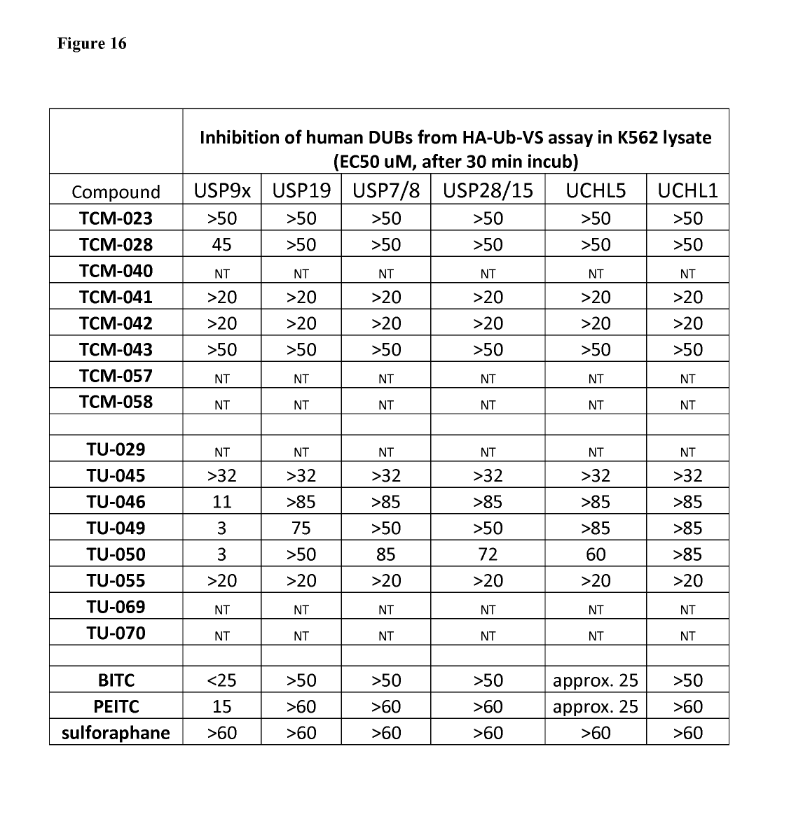

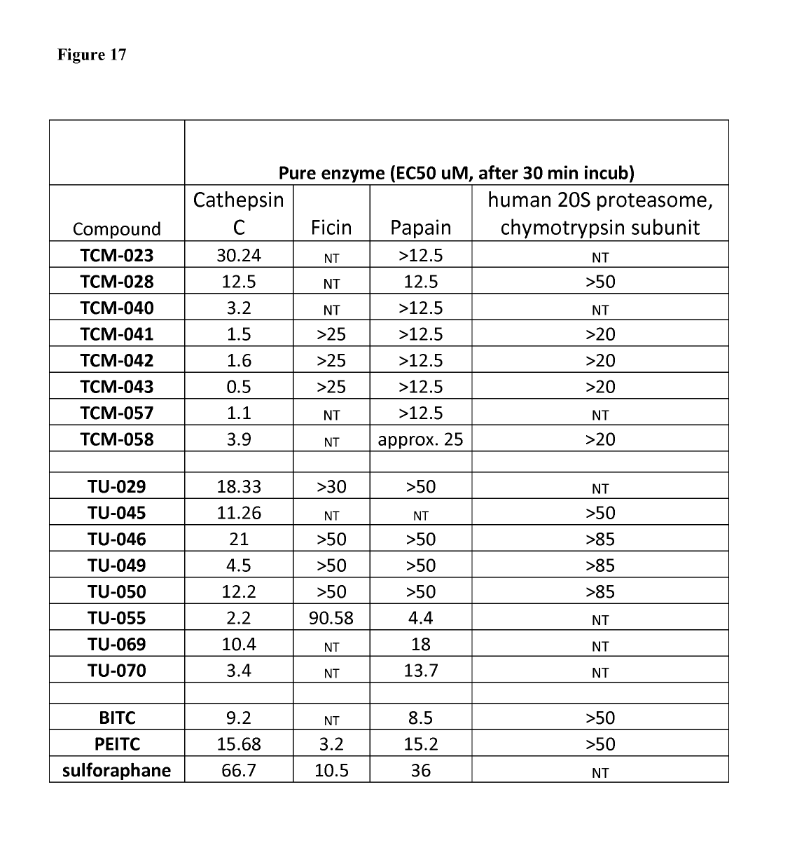

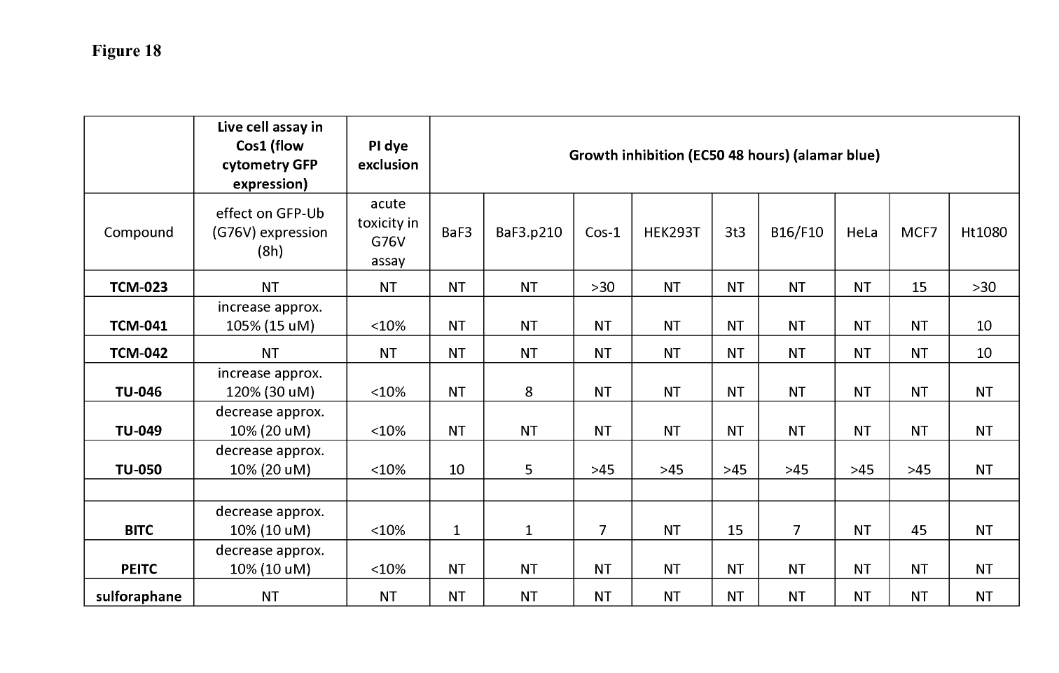

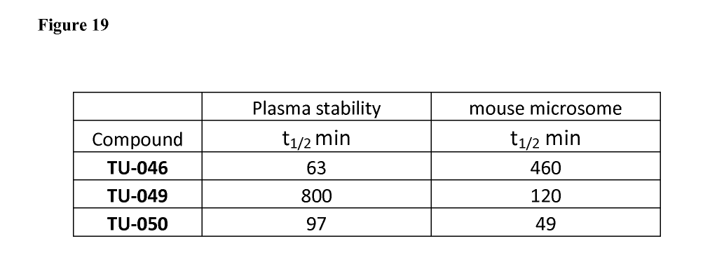

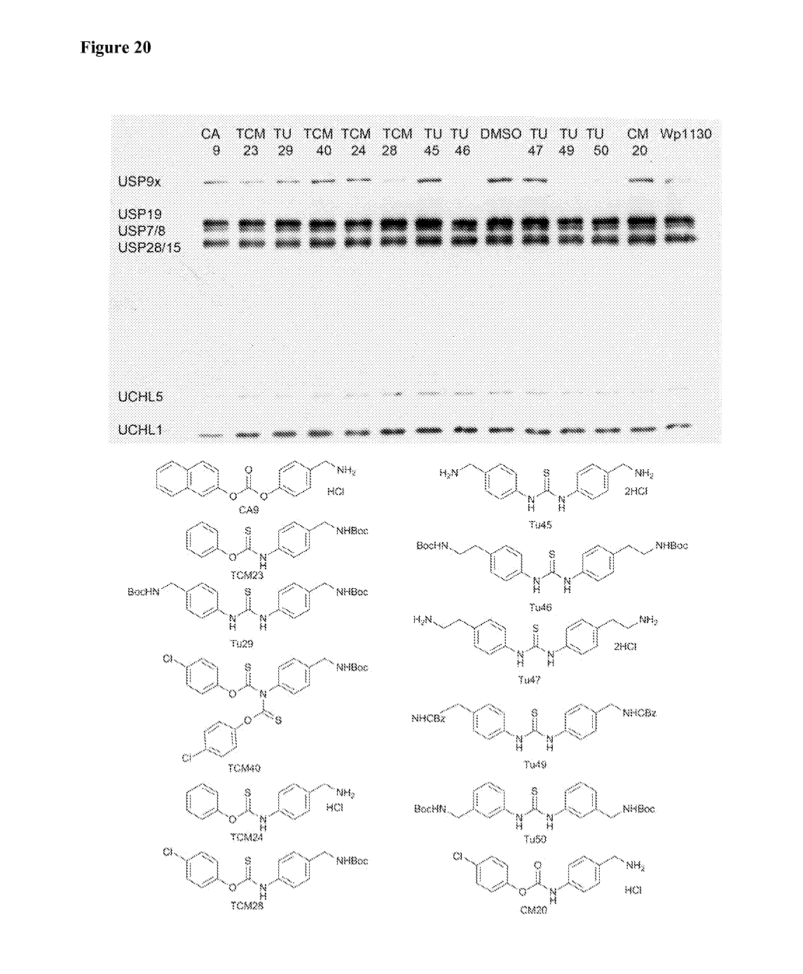

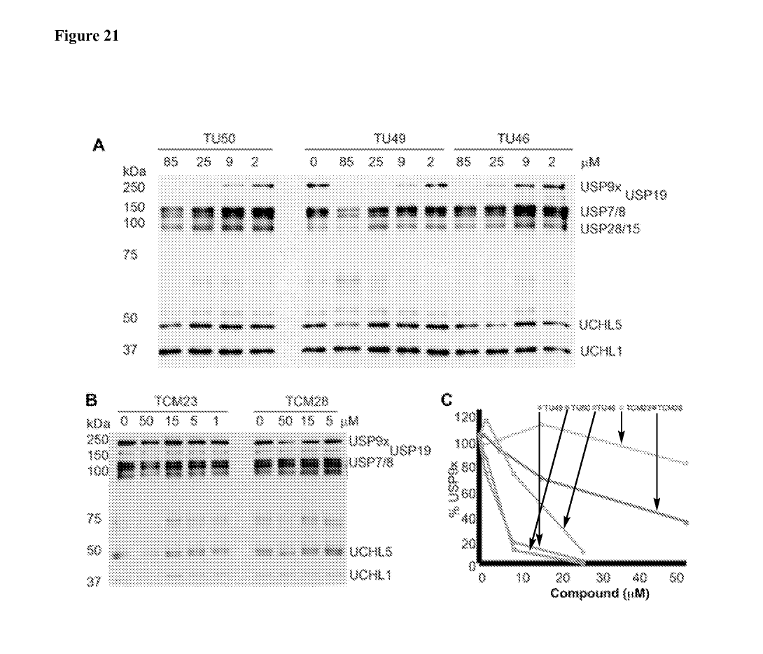

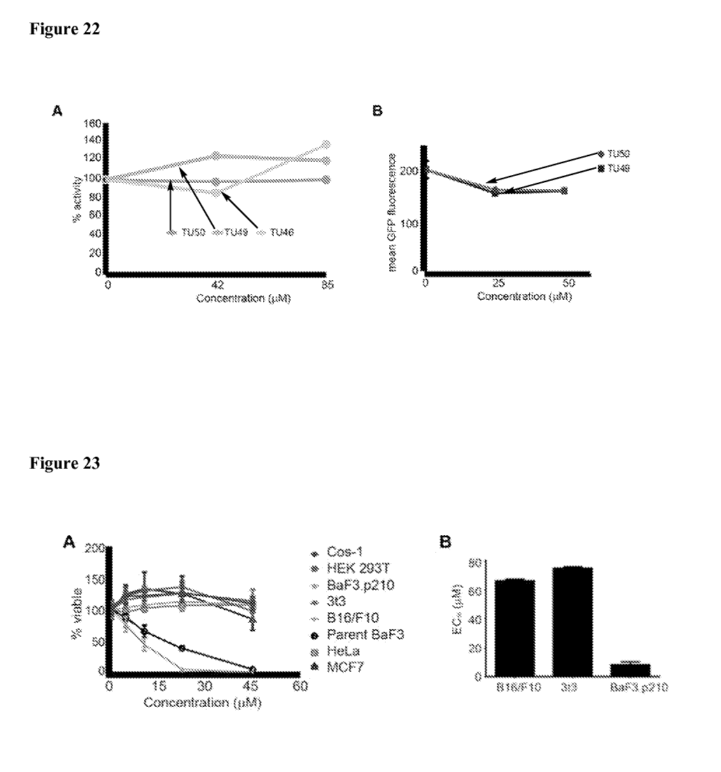

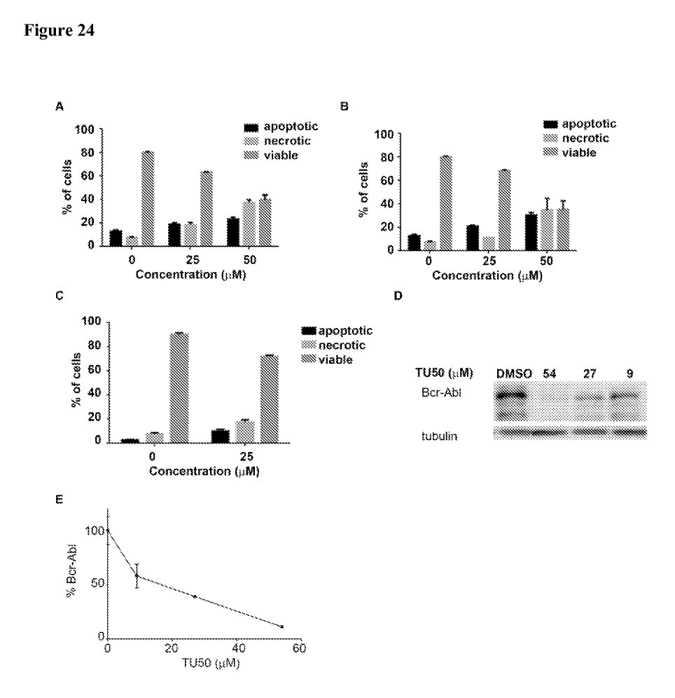

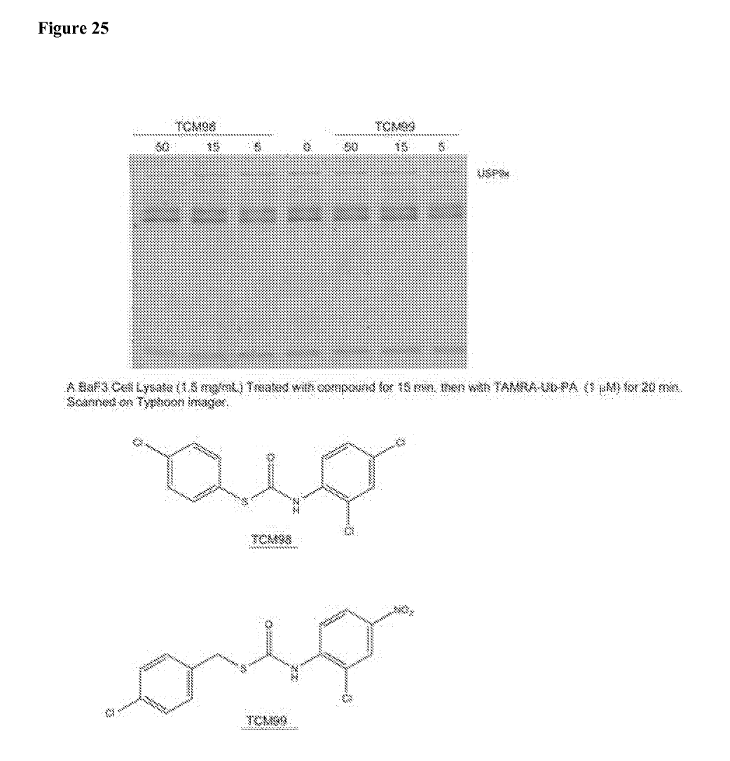

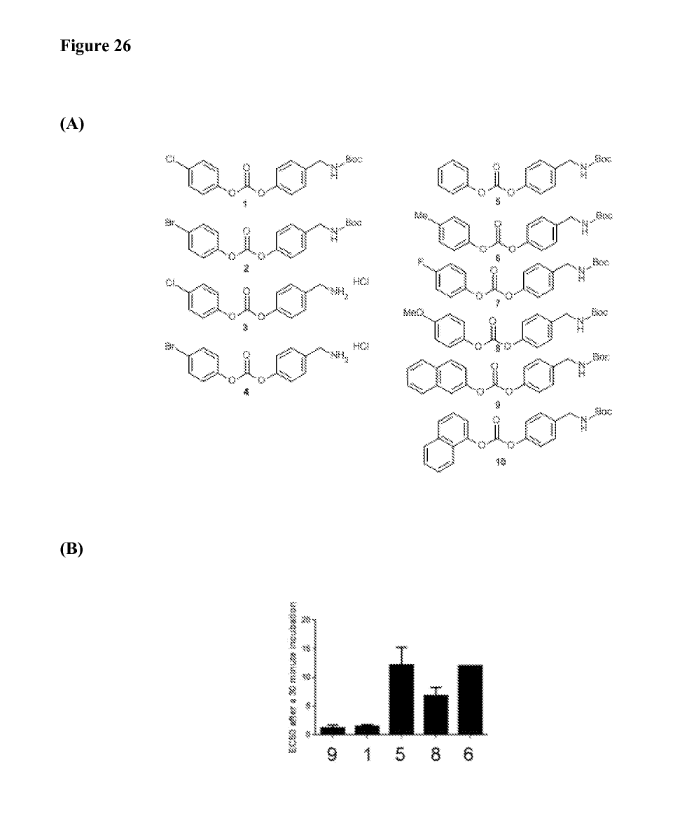

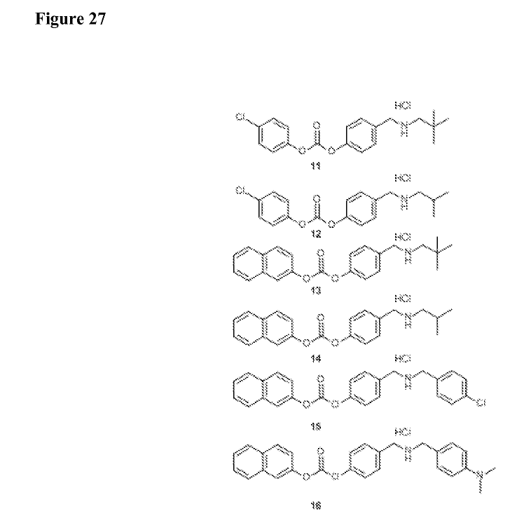

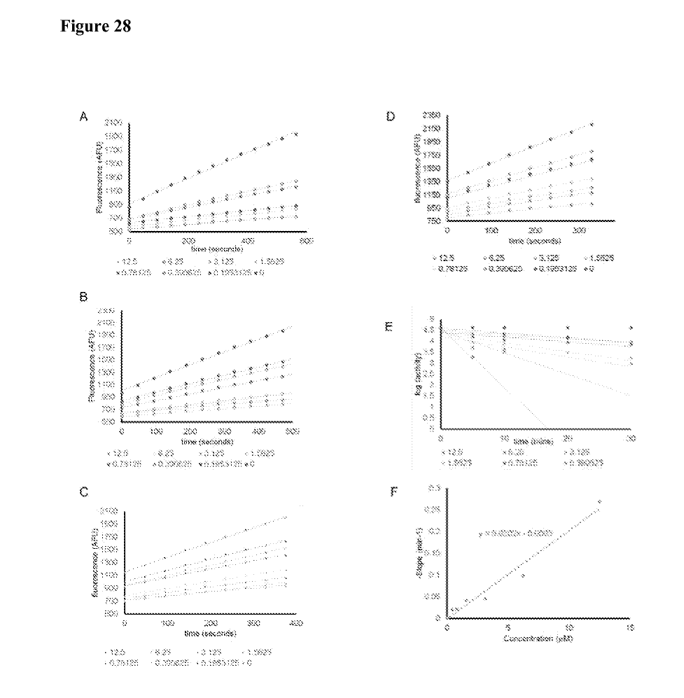

P53 is a tumor suppressor that is rapidly degraded in tumor cells due to ubiquitination by MDM2. In turn, MDM2 is degraded by the ubiquitin/proteasome system. USP7 removes ubiquitin from MDM2, stabilizing the protein, and thereby causing degradation of P53. Inhibition of USP7 consequently causes the degradation of MDM2 and the stabilization of P53. This behavior is observed when cells are treated with 4 (as defined in FIG. 3). Doxorubicin (Doxo) serves as a positive control. See FIG. 11.

### Example 7

### Synthesis of compounds

All reactions were carried out under an atmosphere of dry nitrogen supplied by a balloon. All solvents and amine bases were either distilled before use or bought dry over molecular sieves. All aqueous solutions were saturated unless otherwise stated.

General Procedure 1.

To a double flame dried/vacuum cooled flask, to which had been added 4 Å molecular sieves prior to the first flaming, was added the phenol. This material was placed under a nitrogen atmosphere, dissolved in 3:1 DMF:pyridine (approx. 10 mL to 500 mg phenol) and then the chloroformate was added (either drop wise or via cannula as a solution in DMF over 4 Å sieves if a solid). After 8-15 hours, the liquid phase was decanted off the sieves and water (approx. 0.5 mL per 10 mL) was added to the liquid phase and stirred for 5 mins. After this time, 200 mL each of water and ethyl acetate were added to the mixture and the aqueous phase separated. The organic phase was washed two times with 10% copper (II) sulfate, once with saturated sodium bicarbonate, two times with water and once with brine. The organic phase was then dried over magnesium sulfate, filtered and concentrated in vacuo to yield the crude product.

General Procedure 2.

The Boc protected compound was added to a flame dried flask. The flask was placed under a nitrogen atmosphere and then 2 M HCl in ether was added (100 mL per 500 mg). The reaction was stirred overnight. After this time stirring was stopped, stir bar removed and precipitate allowed to settle. Liquid phase was decanted and fresh ether was added. This cycle was repeated four times, to yield the purified amine as its HCl salt.

General Procedure 3.

The amine and aldehyde were mixed 1:1 in methanol and stirred for 3 hours after which time the mixture was heated to 50° C. for 30 minutes. Reaction mixture was then cooled to 4° C. and sodium borohydride (excess) was added and the reaction mixture left to stir for 1 hour at rt. Reaction mixture was diluted with EtOAc and water was added. Aqueous phase was separated and organic phase extracted 3 times with water then washed with brine. Organic phase was dried over magnesium sulfate, filtered and concentrated in vacuo and crude mixture was used subsequently.

### Synthesis of tert-butyl 4-hydoxybenzylcarbamate

To 4-hyroxybenzylamine (5 g, 40 mmol) in DMF/pyridine (20 mL 5:1) was added Boc2O XS at 4° C. and the reaction was stirred overnight at RT. At this point approx. 0.5 mL 10 M NaOH was added to the reaction and stirring was continued. After 30 minutes 250 ml, water and 200 mL EtOAc were added and the phases separated. The organic layer was washed sequentially with 10% copper sulfate (2 times), sodium bicarbonate and brine. Organic phase was then dried over magnesium sulfate, filtered and concentrated in vacuo. Chromatography on silica gel (elution 60-70% EtOAc in hexane) yielded the purified product as a white solid. δH (400 mHz, CD3SOCD3) 1.144 (9H, s); 3.936 (2H, d, J=5.6 Hz); 6.630 (2H, d, J=8.4 Hz); 6.951 (2H, d, J=8.4 Hz); 7.081 (1H, br s); 9.171 (1H, s).

### Synthesis of tert-butyl(((4-fluorophenoxy)carbonyl)oxy)benzyl carbamate

Following General Procedure 1, tert-butyl 4-hydoxybenzylcarbamate (500 mg, 2.2 mmol) was reacted with p-fluorophenyl chloroformate (0.689 mg, 3.96 mmol) in DMF/pyridine (20 mL). Chromatography on silica gel (gradient from 5% EtOAc in hexanes to 40% EtOAc in hexanes) gave the target compound as a white solid (326 mg, 50%). δH (400 mHz, CD3SOCD3) 1.349 (9H, s); 4.092 (2H, d, J=6.0 Hz); 7.243-7.284 (4H, m); 7.377-7.408 (4H, m).

### Synthesis of 4-(aminomethyl)phenyl(4-fluorophenyl)carbonate

Following General Procedure 2, 15 (100 mg, 0.28 mmol) was dissolved in 2 M HCl in Et2O (20 mL) and stirred overnight at RT. The title compound was obtained as a white solid (50 mg, 60%). δH (400 mHz, CD3SOCD3) 4.008 (2H, s); 7.243-7.284 (4H, m); 7.261 (2H, d, J=8.8 Hz); 7.380-7.409 (4H, m); 7.519 (2H, d, J=8.4 Hz); 8.137 (3H, br s). δC (100 mHz, CD3SOCD3) 44.626; 119.316; 119.553; 124.512; 126.282; 126.384; 135.530; 149.844; 153.647; 154.666; 161.807; 164.219. m/z (ESI+) 262 (100% MH+)

### Synthesis of 4-(aminomethyl)phenyl(phenyl)carbonate

Following General Procedure 2, 14 (90 mg, 0.26 mmol) was dissolved in 2 M HCl in Et2O (20 mL) and stirred overnight at RT. The title compound was obtained as a white solid (36 mg, 50%). δH (400 mHz, DMSO d6) 4.017 (2H, s); 7.294-7.341 (3H, m); 7.524 (2H, d, J=8.4 Hz); 8.165 (3H, s). ESI− (242 M-H+100%)

### Synthesis of tert-butyl(((4-bromophenoxy)carbonyl)oxy)benzyl carbamate

Following General Procedure 1, tert-butyl 4-hydoxybenzylcarbamate (500 mg, 2.2 mmol) was reacted with p-bromophenyl chloroformate (0.931 mg, 3.96 mmol) in DMF/pyridine (20 mL). Chromatography on silica gel (gradient from 5% EtOAc in hexanes to 40% EtOAc in hexanes) gave the target compound as a white solid (509 mg, 55%). m/z (ESI+) 440 (100% MNH4+)

### Synthesis of 4-(aminomethyl)phenyl(4-bromophenyl)carbonate

Following General procedure 2, 17 (150 mg, 0.36 mmol) was dissolved in 2 M HCl in Et2O (20 mL) and stirred overnight at RT. The title compound was obtained as a white solid (51 mg, 40%). δH (400 mHz, CD3OD) 4.100 (2H, s); 7.216 (2H, d, J=4.8 Hz); 7.364 (2H, d, J=8.8 Hz); 7.573 (2H, d, J=4.8 Hz). δC (100 mHz, CD3OCD3) 44.565; 121.971; 124.459; 126.763; 123.653; 135.621; 135.690; 152.972; 153.636; 154.330.

### Synthesis of N-Boc 4-(aminomethyl)phenyl(4-chlorophenyl)carbonate

Following General Procedure 1, tert-butyl 4-hydoxybenzylcarbamate (500 mg, 2.2 mmol) was reacted with p-chlorophenyl chloroformate (0.931 mg, 3.96 mmol) in DMF/pyridine (20 mL). Chromatography on silica gel (gradient from 5% EtOAc in hexanes to 40% EtOAc in hexanes) gave the target compound as a white solid (509 mg, 55%). δH (400 mHz, CD3SOCD3) 1.355 (9H, s); 4.102 (2H, d, J=4.8 Hz); 7.277-7.405 (4H, m); 7.490 (2H, d, J=6.8 Hz); 7.507 (1H, d, J=6.8 Hz). δC (100 mHz, CD3SOCD3) 31.332; 45.893; 124.077; 126.359; 131.227; 132.722; 133.729; 141.021; 152.423; 154.559; 158.885. m/z (ESI+) 378 (100% MH+).

### Synthesis of 4-(aminomethyl)phenyl(4-chlorophenol)carbonate

Following General procedure 2, 16 (110 mg, 0.29 mmol) was dissolved in 2 M HCl in Et2O (20 mL) and stirred overnight at RT. The title compound was obtained as a white solid (54 mg, 60%). δH (400 mHz, CD3SOCD3) 3.995 (2H, s); 7.381-7.413 (4H, m); 7.506 (2H, d, J=8.8 Hz); 7.580 (2H, d, J=8.8 Hz), 8.537 (3H, s). δC (100 mHz, DMSO d6) 44.596; 124.482; 126.374; 132.745; 133.645; 133.783; 135.606; 152.491; 153.651; 154.391. m/z (ESI+) 279 (100% MH+).

### Synthesis of 4-(aminomethyl)phenyl(2-chlorophenol)carbonate

Following General procedure 2, the corresponding Boc protected species (55 mg, 0.15 mmol) was dissolved in 2 M HCl in Et2O (10 mL) and stirred overnight at RT. The title compound was obtained as a white solid (20 mg, 44%). δH (400 mHz, CD3OD) 4.133 (2H, s); 7.216 (2H, d, J=4.8 Hz); 7.364 (2H, d, J=8.8 Hz); 7.573 (2H, d, J=4.8 Hz). δC (100 mHz, CD3SOCD3) 44.581; 124.337; 126.885; 128.632; 131.516; 131.890; 133.477; 133.790; 135.820; 149.455; 153.552; 153.704. m/z (ESI−) 276 (100% M-H+)

### Synthesis of N-Boc 4-(aminomethyl)phenyl(2-napthyl)carbonate

Following General Procedure 1, tert-butyl 4-hydoxybenzylcarbamate (500 mg, 2.2 mmol) was reacted with 2-napthyl chloroformate (0.803 mg, 3.96 mmol) in DMF/pyridine (20 mL). Chromatography on silica gel (gradient from 5% EtOAc in hexanes to 40% EtOAc in hexanes) gave the target compound as a white solid (509 mg, 55%). δH (400 mHz, CD3SOCD3) 1.348 (9H, s); 4.099 (2H, d, J=6.0 Hz); 7.290-7.322 (4H, m); 7.35 (1H, m); 7.497-7.539 (4H, m); 7.875 (1H, d, J=2.0 Hz); 7.925 (1H, dd, J=8.2, 4.0 Hz); 7.990 (1H, d, J=8.8 Hz).

### Synthesis of N-Boc 4-(aminomethyl)phenyl(2-napthyl)carbonate

Following General procedure 2, 19 (200 mg, 0.51 mmol) was dissolved in 2 M HCl in Et2O (40 mL) and stirred overnight at RT. The title compound was obtained as a white solid (67 mg, 40%). δH (400 mHz, CD3SOCD3) 4.023 (2H, s); 7.444 (2H, d, J=8.4 Hz); 7.533-7.551 (5H, m); 7.865-8.014 (4H, m); 8.213 (3H, s). m/z (ESI+) 294 (100% MH+).

### Synthesis of 4-(aminomethyl)phenyl(4-methoxyphenyl)carbonate

Following General procedure 2, the corresponding Boc protected compound (160 mg, 0.43 mmol) was dissolved in 2 M HCl in Et2O (30 mL) and stirred overnight at RT. The title compound was obtained as a white solid (60 mg, 45%). δH (400 mHz, CD3SOCD3) 3.751 (3H, s); 4.052 (2H, s); 6.964 (2H, d, J=8.2 Hz); 7.252 (2H, d, J=8.2 Hz); 7.402 (2H, d, J=8.5 Hz); 7.522 (2H, d, J=8.5 Hz); 8.215 (3H, s). δC (100 mHz, CD3SOCD3) 160.404; 155.017; 153.743; 147.242; 135.469; 133.607; 125.237; 124.527; 117.668; 58.604; 44.603. m/z (ESI+) 274 (100% MH+).

### Synthesis of 4-(aminomethyl)phenyl(4-methylphenyl)carbonate

Following General procedure 2, the corresponding Boc protected compound (100 mg, 0.28 mmol) was dissolved in 2 M HCl in Et2O (20 mL) and stirred overnight at RT. The title compound was obtained as a white solid (24 mg, 30%). δH (400 mHz, CD3SOCD3) 2.278 (3H, s); 4.006 (2H, s); 7.186-7.216 (4H, m); 7.387 (2H, d, J=8.4 Hz); 7.535 (2H, d, J=8.5 Hz); 8.295 (3H, s). δC (100 mHz, CD3SOCD3) 23.476; 44.588; 123.993; 124.527; 133.111; 133.615; 135.492; 138.902; 151.606; 153.712; 154.796. m/z (ESI+) 258 (100% MH+).

### Synthesis of 4-chlorobenzyl 4-hydroxybenzylamine

4-chlorobenzaldehyde and 4-hydroxybenzylamine were mixed 1:1 in methanol (4 mL) and the reaction was stirred for 1 hour at room temp followed by a further hour at reflux. After cooling to 4° C. and dilution into 20 mL total methanol, sodium borohydride (3 equivalents) was added portionwise over 1 hour. The reaction was allowed to stir for a further hour, after which time 200 mL EtOAc was added and 250 mL water. Phases were separated and the organic layer was washed 3 times with sodium bicarbonate and then with brine. Organic layer was dried with magnesium sulfate, filtered and concentrated to give the crude amine which was used without further purification. δH (400 mHz, CD3OD) 3.594 (2H, s); 3.664 (2H, s); 7.711 (2H, d, J=8.8 Hz); 7.107 (2H, d, J=8.8 Hz); 7.282-7.295 (4H, m).

### Synthesis of N-Boc 4-chlorobenzyl 4-hydroxybenzylamine

4-chlorobenzyl 4-hydroxybenzylamine was dissolved in DMF/pyridine (20 mL 5:1) was added Boc₂O XS at 4° C. and the reaction was stirred overnight at RT. At this point approx. 0.5 mL 10 M NaOH was added to the reaction and stirring was continued. After 30 minutes 250 mL water and 200 mL EtOAc were added and the phases separated. The organic layer was washed sequentially with 10% copper sulfate (2 times), sodium bicarbonate and brine. Organic phase was then dried over magnesium sulfate, filtered and concentrated in vacuo. Chromatography on silica gel (elution 30-40% EtOAc in hexane) yielded the purified product as a white solid. (note: peaks are broad due to rotameric equilibria about the N-Boc bond). δH (400 mHz, CD3OD) 1.399; 4.195 (4H, br s); 6.664 (2H, d, J=8.4 Hz); 6.979 (2H, d, J=7.8 Hz); 7.156 (2H, m); 7.282-7.340 (2H, d, J=8.4 Hz).

### Synthesis of 4-(((4-chlorobenzyl)N-Boc amino)methyl)phenyl phenyl carbonate

Following General procedure 2, the corresponding Boc protected compound was dissolved in 2 M HCl in Et₂O (30 mL) and stirred overnight at RT. The title compound was obtained as a white solid. δH (400 mHz, CD₃SOCD₃) 1.339 (9H, s); 4.316 (4H, br s); 7.274-7.366 (1H, br s); 6.979 (11H, m); 7.441 (2H, t, J=8.0 Hz).

### Synthesis of 4-(((4-chlorobenzyl)amino)methyl)phenyl phenyl carbonate

Following General procedure 2, the corresponding Boc protected compound (100 mg, 0.22 mmol) was dissolved in 2 M HCl in Et₂O (20 mL) and stirred overnight at RT. The title compound was obtained as a white solid (30 mg, 35%). δH (400 mHz, CD₃SOCD₃) 4.145 (4H, br s); 7.279-7.560 (13H, m); 9.481 (2H, br s). δC (100 mHz, CD₃SOCD₃) 52.256; 124.329; 124.543; 129.662; 131.639; 132.844; 133.410; 134.042; 134.813; 135.240; 136.758; 153.727; 154.040; 154.605. m/z (ESI+) 368 (100% MH+).

### Synthesis of 4-(pent-4-ynamidomethyl)phenyl phenyl carbonate

Following General procedure 2, the corresponding Boc protected compound (100 mg, 0.22 mmol) was dissolved in 2 M HCl in Et₂O (20 mL) and stirred overnight at RT. The title compound was obtained as a white solid.

### Synthesis of 4-chlorophenyl(4-(hex-5-ynamidomethyl)phenyl)carbonate

Following General procedure 2, the corresponding Boc protected compound was dissolved in 2 M HCl in Et₂O (20 mL) and stirred overnight at RT. The title compound was obtained as a white solid.

### Synthesis of carbamate 22

Following General procedure 2, the corresponding Boc protected compound was dissolved in 2 M HCl in Et₂O (20 mL) and stirred overnight at RT. The title compound was obtained as a white solid. δH (400 mHz, CD₃SOCD₃) 3.903 (2H, s); 7.180 (2H, d, J=7.6 Hz); 7.402 (4H, m); 7.493 (2H, d, J=7.6 Hz); 8.407 (3H, s), 10.319 (1H, s). δC (100 mHz, CD3SOCD3) 44.825; 121.483; 125.023; 128.587; 131.555; 132.531; 132.852; 141.901; 153.537; 154.803. m/z (ESI+) 243 (100% MH+).

### Synthesis of 4-(aminomethyl)phenyl 4-chlorobenzoate

Ester was prepared by EDCI coupling of the corresponding alcohol with the corresponding carboxylic acid. m/z (ESI+) 262 (100% MH+).

### Synthesis of 4-(aminomethyl)phenyl benzoate 26

m/z (ESI+) 228 (100% MH+)

### Synthesis of 31

According to general procedure 1, isobutyl chloroformate was reacted with tert-butyl 4-hydoxybenzylcarbamate in DMF:pyridine. Purification by chromatography on silica gel yielded the Boc protected intermediate. Then according to general procedure 2, the Boc product was treated with 2 M HCl in Et₂O to yield the title compound. δH (400 mHz, CD₃SOCD₃) 0.876 (6H, d, J=6.5); 1.921 (1H, m); 3.95 (2H, d, J=6.8); 7.209 (2H, d, J=7.6 Hz); 7.533 (2H, d, J=7.6), 8.645 (3H, s). δC (100 mHz, CD₃SOCD₃) 21.813; 30.328; 44.527; 77.328; 124.436; 133.125; 135.125; 153.758; 156.156. m/z (ESI+) 224 (100% MH+).

### Example 8

### General Materials and Methods for Example 9

### Materials

All chemicals and reagents were from Sigma Aldrich unless otherwise stated. Bortezomib was from LC laboratories (Woburn, Mass.). Solvents were from Fisher (Pittsburgh, Pa.). G5 isopeptidase inhibitor 1 (50-230-7928) was from Calbiochem (Philadelphia, Pa.). Diphenylcarbonate and ditolylcarbonate were from Alfa Aesar (Ward Hill, Mass.). Alamar Blue® was from Invitrogen (Grand Island, N.J.). Ubiquitin aldehyde, HA-ubiquitin vinylsulfone, ubiquitin vinylsulfone, NSC 632839 hydrochloride and LDN 54777 were from Boston Biochem (Cambridge, Mass.). Boc₂O, 2-naphthyl chloroformate, water soluble carbodiimide and HATU were from TCI America (Portland, Oreg.). Column chromatography was performed on silica gel (Siliaflash, Silicycle, Quebec, Canada) and TLC was performed on SiliaPlates and visualized by UV. NMR spectroscopy (¹H) was performed on a Bruker 400 MHz instrument in D₃CSOCD₃, CD₃OD, or CDCl₃. Deuterated solvents were purchased from Cambridge Isotope Laboratories (Cambridge, Mass.). DMEM, glutamax, penicillin/streptomycin were from Gibco (Grand Island, N.J.). Trypsin (0.25%) was from Hyclone (Logan, Utah). Bradford dye and Chill-out wax were from BioRad (Hercules, Calif.). USP 7 inhibitor P005091 was from RnD Systems (Minneapolis, Minn.). Dithiothreitol reagent was from Gold Biotech (St Louis, Mo.). ECL II was from Pierce (Rockland, Ill.). Blue Biofilm was from Denville Scientific (Metuchen, N.J.). PVDF was from Millipore (Billerica, Mass.). LC/MS was performed on a Waters Acuity Ultra Performance LC with Waters MICROMASS detector. Antibodies: anti-K48-linked ubiquitin, clone APU2; anti-K63-linked ubiquitin, clone APU3, were from Millipore (Billerica, Mass.); anti-SMAD4, H-552; anti-Mdm2, SC-13161 were from Santa Cruz (Santa Cruz, Tex.); anti-PARP, 9542; anti-Abl, 2862; β-tubulin, 2156 were from Cell Signaling Technologies (Beverley, Mass.). Anti-actin was clone AC-40, A3853 and anti-GAPDH was clone G9295. Anti-HA Clone 3F10 was from Roche (Indianapolis, Ind.). HRP conjugated secondary antibodies were from AbCam (Cambridge, Mass.).

### Vehicle

All compounds were administered as solutions in DMSO. For in vitro assays final DMSO concentration was 1%. For cell culture studies, final DMSO concentration was 0.1%.

### Tissue Culture Assays

All cells were grown at 37° C. in a 5% CO₂ humidified atmosphere in DMEM supplemented with 10% heat inactivated FBS, 1× glutamax, and 1× penicillin/streptomycin. Cells were transfected using Mirus 2020 (Madison, Wis.) as per the manufacturer's instructions. For G76V assay, confluence at transfection was approximately 75% whereas for HA-ubiquitin 50-60% was used. Transfected cells were harvested 1.5 days post transfection. Prior to harvesting, medium was replaced with fresh medium containing either 0.1% DMSO or 0.1% DMSO plus compound. Cells were harvested after 2-8 hours by aspiration of media, trypsinization, resuspension in complete media, centrifugation 700 g, and washing 3 times in PBS. Cells were lysed using 3× freeze thaw cycles in 75 mM potassium phosphate pH 7.5, 150 mM NaCl (lysis buffer) with protease inhibitors then centrifuged at 20 000 rpm (microcentrifuge, Eppendorf 5417 C) for 10 minutes. Typically clarified lysate was analyzed. Where indicated, SDS was added to the pellet and supernatant and this mixture was sonicated and centrifuged (20,000 rpm, microcentrifuge, Eppendorf 5417 C) to give a whole cell lysate. When studying Bcr-Abl, 10 mM HEPES (pH 7.9), 5 mM MgCl₂, 140 mM KCl, 1% NP40, protease inhibitors and Phosphatase Inhibitor Cocktail II was used as the lysis buffer. Lysates were centrifuged (20 000 rpm, microcentrifuge, Eppendorf 5417 C) concentration was measured, then 0.1% SDS was added and lysates were sonicated for a total of 30 s (in 10 s bursts). Protein concentration was determined using Bradford assay with IgG as standard and analyzed by western blot as delineated below {[9 μg total protein for K48-linked ubiquitin (1:9000 antibody dilution), SMAD4 (1:500) or PARP (1:1000)]; [30-40 μg was loaded for K63-linked ubiquitin (1:1500), Mdm2 (1:1000) or Abl (1:1500). Signals were normalized to actin (1:10000 for 9 μg lysate; 1:25000 for 30-40 μg lysate), β-tubulin (1:8000) or GAPDH (1:35000)]}. Proliferation assays were conducted by plating cells at 5% confluence in 96 well plates together with compound or 0.1% DMSO. Cells were allowed to grow for 72 hours (a 24 h dosing regimen was used for carbonate compounds) and then Alamar Blue® was added and number of cells was measured by fluorescence on a microplate reader.

### FACS Analysis

FACS was carried out on a Beckman FACS-Calibur. For HEK 293T and K562, cells were resuspended by repeated pipetting/agitation of the incubation media, followed by dilution into PBS. For Cos-1, MCF-7, and CHO cells, media was removed and trypsin was added. Harvested cells were placed in FACS buffer (0.5% FBS in PBS with 3 μg/mL propidium iodide) 30 s prior to analysis. All data were analyzed using FlowJo V10, from TreeStar (Ashland, Oreg.). Approximately 2500 cells were sorted per replicate. Cells were sorted by propidium iodide dye exclusion to give a “viable population”. GFP positive cells within this group were identified relative to untransfected controls. Then the geometric mean of the whole GFP positive population within the viable population was calculated. Typical transfection efficiencies for G76V ubiquitin were 60-70% for both Cos-1 and HEK 293T and 25-40% for CHO cells, based on GFP positive cells.

### Lysate Assays

Cells overexpressing HA-ubiquitin were prepared as above. Pellets were typically stored at −80° C. until required, at which time they were thawed on ice. Cell lysis was performed in lysis buffer using a Dounce homogenizer (10 strokes, with grinding, on ice: typical yield approx. 2-5 mg protein per transfected T75 flask for HEK 293T cells; 1-3 mg protein from a T75 flask for Cos-1). Crude lysate was centrifuged at 17000 g for 10 min at 4° C., after which time the concentration of the lysate was normalized to 1 mg/mL. The lysate was aliquoted into PCR strip tubes (typical volumes 75-50 μL) and compound in DMSO was added to this to give a final concentration of DMSO of 1%. Tubes were briefly centrifuged, overlaid with Chill-out wax (50 μL) and placed in a PCR machine at 37° C. with heated lid set to 37° C. Aliquots (9 μL) were removed at the stated times and immediately quenched in (2× final concentration) reducing (dithiothreitol) loading buffer and frozen (−19° C.) till required. Western blot analysis was carried out using standard methods. Samples were resolved by SDS-PAGE, transferred to PVDF [(0.45 μm) (Towbin buffer, tank apparatus, 90 V 1 hour, then overnight at 30 V, 4° C.)] then blocked in 15% milk in TBS-T HS (100 mM Tris HCl, pH 7.6, 500 mM NaCl, 0.5% Tween-19) for at least 2 hours at RT. Afterward, membrane was washed in TBS-T HS then probed with anti HA-HRP (1:18000) for 1.33 hours at RT. Membrane was washed 3 times in TBS-T HS (15 mins) then once in TBS (15 mins) and exposed to ECL II and visualized using blue biofilm. The dynamic range of the assay at the 2 hour time point was approximately 5 for HEK 293T and 2.5 for Cos-1 cell lysates, which showed the same trend as observed for HEK 293T cells. When required, membranes were stripped in 100 mM glycine pH 4, 500 mM NaCl, 1% SDS, 5 mM BME, at 55° C. for 19 mins, then analyzed.

HA-ubiquitin-vinylsulfone activity profiling

Lysate labeling assay on untransfected cells (1.5 mg/mL) was run with the stated concentration of inhibitor (or 1% DMSO control) for between 19-60 mins. After this time HA-Ub-VS (1.5-0.7 μM) was added and incubated for 19 mins. After this time reaction mixture (9 μL) was removed and quenched in 2× (final concentration) reducing loading buffer. For recovery experiments, a lysate of 6 mg/mL was treated with saturating compound C14 (250 μM) and incubated for 40 mins. Afterward, the lysate was diluted to 0.6 mg/mL (final concentration of inhibitor 25 μM) in lysis buffer (final volume 100 μL), then HA-Ub-VS was added. Aliquots (15 μL) were removed at the stated time (5-119 mins) and immediately quenched in 2× (final concentration) reducing loading buffer. For all HA-Ub-VS experiments samples in loading buffer were heated only to 37° C. prior to loading on a gel. This assay is highly susceptible to concentration of lysate and HA-Ub-VS. Cell experiments were carried out as above with some modifications. For Cos-1 and MCF-7 cells, after trypsinization, media with compound was added to give a final concentration of compound equal to that used in the assay. Cell pellets were lysed on an ice/salt bath with a temperature of −5° C. and lysate was centrifuged for only 5 mins.

### Enzyme Assays

Enzyme was preincubated for 30 min at 25° C. with inhibitor prior to addition of substrate. The release of AMC was measured by monitoring the change in fluorescence (excitation wavelength 360 nm, emission wavelength 460 nm) every 47 sec using a Biotek plate reader for 30 minutes. The final concentration of DMSO in all assays was 2%. The concentration of compound required to inhibit the enzyme by 50% was calculated using Prism Prism (GraphPad Software Inc., La Jolla, Calif., using the equation: activity=1/(1+([inhibitor]/IC₅₀))). Ficin and papain (8 μg/mL) were assayed in 100 mM potassium phosphate, pH 6.8, 0.4 mM β-mercaptoethanol with the substrate Z-Arg-AMC (300 μM) (BaChem, Torrance, Calif.).

### Example 9

### Inhibition of DUBs

### The Methylamino Diphenylcarbonate C4 is a Broad Spectrum DUB Inhibitor

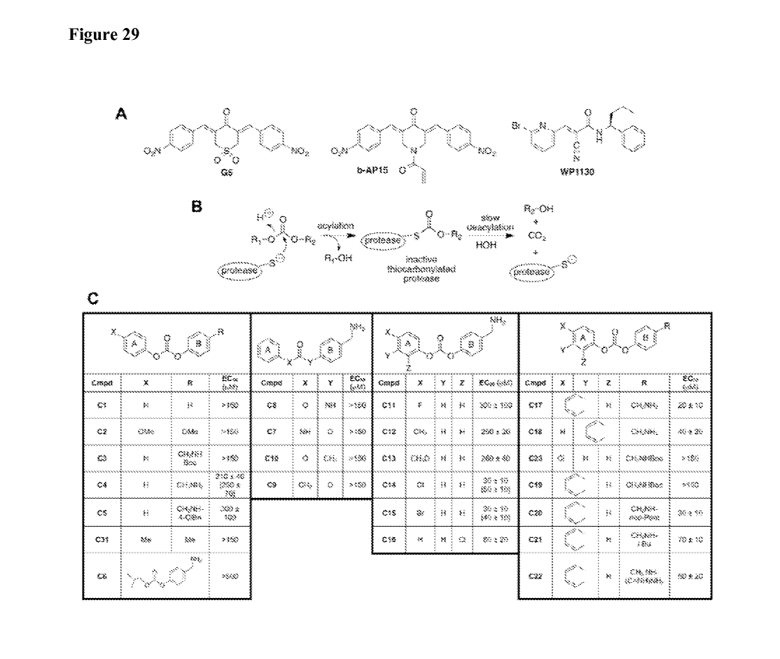

Carbonate esters inhibit chymotrypsin by forming a stable carbonylated enzyme that mimics the acylenzyme intermediate formed during the catalytic cycle. To investigate whether carbonate esters might similarly inhibit cysteine proteases via an analogous reaction to form a stable thiocarbonate (FIG. 29B), a small set of diphenyl carbonates was screened (compounds C1-C6, FIG. 29C) by monitoring the accumulation of high molecular weight ubiquitinated proteins (HMW-Ub).

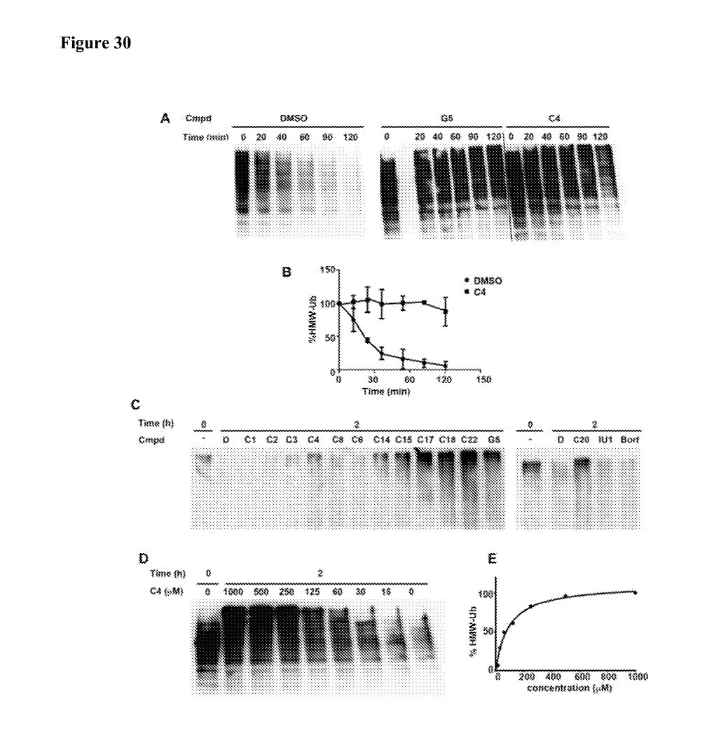

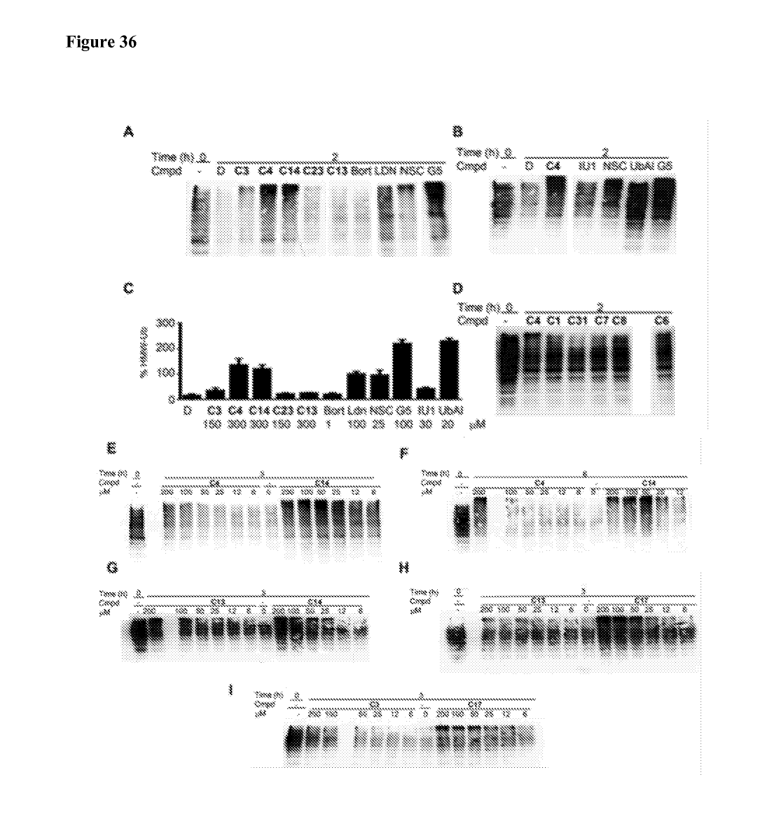

Lysates were prepared from HEK 293T cells expressing N-terminally HA-tagged ubiquitin (HA-Ub) to facilitate the observation of ubiquitinated proteins. In the absence of a DUB inhibitor, the HMW-Ub pool decomposed with a half-life of 34 min (FIG. 30A,B). The pan-DUB inhibitor G5 isopeptidase inhibitor I (G5) stabilized the HMW-Ub pool (FIG. 30A). G5 also caused the accumulation of HMW-Ub species that were not observed in untreated lysates, suggesting that additional ubiquitin conjugation occurred during the incubation. Similar stabilization of HMW-Ub was observed with two other pan-DUB inhibitors, ubiquitin-aldehyde and LDN 54777 (FIG. 36). In contrast, the proteasome inhibitor bortezomib failed to stabilize the HMW-Ub pools in these lysates (FIG. 36). Similar results were obtained in lysates prepared from Cos-1 cells expressing HA-Ub. These observations demonstrate that the stabilization and accumulation of HMW-Ub can be used to screen for DUB inhibition.

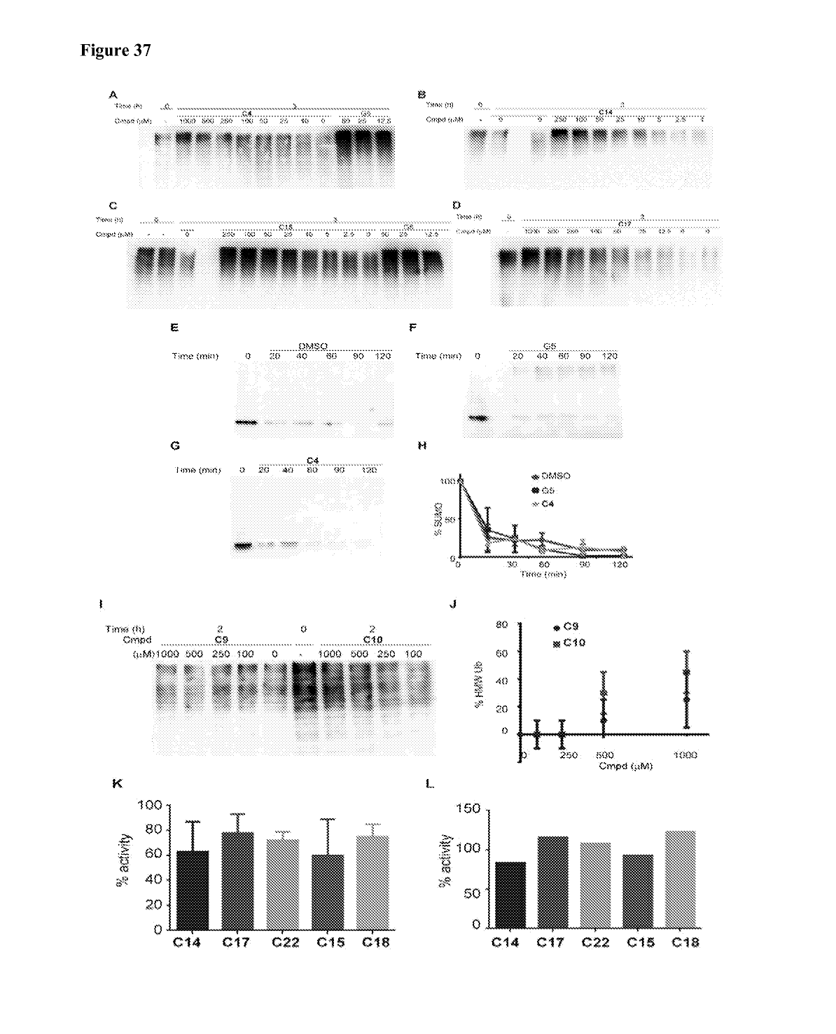

Compounds C1-C3, C6 and C31 failed to substantially stabilize the HMW-Ub pool. The methylamino diphenylcarbonate C4 (500 μM) prevented decomposition of the HMW-Ub pool, increasing half-life to ≧150 min (FIG. 30A). Like G5, this compound caused the accumulation of new HMW-Ub species. Compound C5 also inhibited the decomposition of the HMW-Ub pool and caused the accumulation of new HMW-Ub species. These effects were dose-dependent, with values of EC₅₀ of 210 04 and 310 04 for C4 and C5, respectively, after 2 h incubation (FIG. 30C-E). Similar effects were observed when the endogenous K48-linked ubiquitin pool was monitored in lysates prepared from wild-type HEK 293T cells (FIG. 37A-D). Unfortunately, K63-linked ubiquitin could not be detected in these lysates. The ability of C4 to stabilize SUMOylated proteins in lysates from HA-SUMO transfected HEK 293T cells was also assessed. Neither G5 nor C4 inhibited desumoylation (FIG. 37E-H). These results establish C4 and C5 as new DUB inhibitors.

### Structure-Activity Relationship Study of C4

The importance of the carbonate functionality in DUB inhibition was evaluated. Neither the analogous carbamates (C7 and C8, FIG. 30 and FIG. 36), nor the analogous esters (C9 and C10) stabilized HMW-Ub (FIG. 3714), indicating that the carbonate is required for DUB inhibition.

The structure activity relationship (SAR) of the A ring was also investigated. The p-F (C11), p-Me (C12) and p-MeO (C13) substitutions had no effect on inhibitory activity, suggesting that this position does not interact directly with the DUBs (FIG. 29). In contrast, the p-Cl (C14) and p-Br (C15) substitutions increased inhibitory potency by a factor of approximately 10. The half-life of HMW-Ub pools in HEK 293T cell lysates treated with C14 (250 μM) was >6 h (FIG. 36F). The superiority of p-Cl over the isosteric p-Me substitution also suggests that electronic properties, rather than steric interactions, account for the improved activity of C14 and C15. The p-Cl and p-Br groups are more electron withdrawing than the other three substitutions (pKa≦9.4 for the corresponding p-Cl and p-Br phenols versus pKa≧9.9 for the unsubstituted, p-F, p-Me and p-MeO phenols). The o-Cl (C16), 1-naphthyl (C17) and 2-naphthyl (C18) analogs also displayed improved potency relative to C4. These groups are more electron-withdrawing than p-Me (pKa≦9.5 for the corresponding phenol/naphthols). Addition of electron withdrawing substituent makes the A ring phenol/naphthol a much better leaving group than the B ring phenol, which might suggest that the inactivated enzymes are methylaminophenylthiocarbonylated. However, these substitutions also activate the carbonyl for attack by the cysteine nucleophile, so formation of alternative phenylthiocarbonylated enzymes cannot be ruled out. It is possible that some DUBs react to form phenylthiocarbonylated enzymes while others form methylaminophenylthiocarbonylated enzymes.

The screening results suggested that the amine functionality of ring B is required for activity (FIG. 29C). Further exploration of the SAR of the B ring phenol confirmed this finding. Modification of the amino group with a benzyl (C5) retained DUB inhibitory activity, while activity was lost with Boc modification (C3). Inhibitory activity was not recovered when the A ring phenol contained p-Cl (C23) or was replaced with naphthol (C19). In contrast, inhibitory activity was retained with neopentyl substitution (compare C20 to C17), but isobutyl substitution was somewhat deleterious (C21). Lastly, replacement of the amine with guanidinium was also efficacious (C22).

The ability of diphenylcarbonates to inhibit the cysteine proteases papain and ficin was also tested. None of the compounds was an effective inhibitor of either enzyme (FIG. 37K-L).

### Diphenyl Carbonates are Broad Spectrum DUB Inhibitors

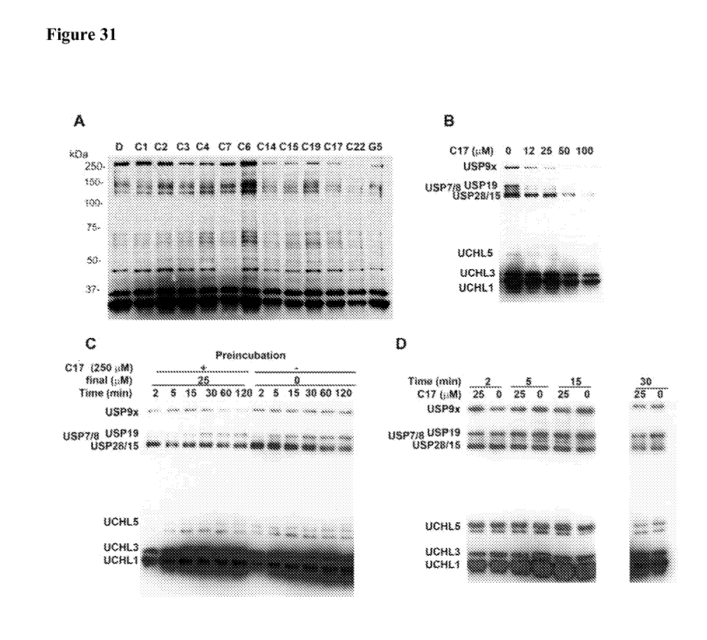

HA-Ub-VS is an irreversible inhibitor of DUBs that is widely used in activity profiling. If the DUB inhibitors react to form a thiocarbonylated enzyme as proposed (FIG. 29B), then HA-Ub-VS labeling will be blocked. Treatment of HEK 293T lysates with HA-Ub-VS produced the characteristic pattern of protein bands at 250, 150-100, 45, 38 and 36 kDa, generally ascribed to USP9x (292 kDa), USP19 (146 kDa), USP7/8 (128 and 127 kDa, respectively), USP28/15 (122 and 112 kDa, respectively), UCH-L5 (38 kDa), UCH-L3 (26 kDa) and UCH-L1 (25 kDa) as depicted in FIG. 31. As expected, preincubation with G5 decreased the labeling of all the USPs and UCH-L1 but not UCH-L3, confirming that this assay can be used to profile DUB inhibition.

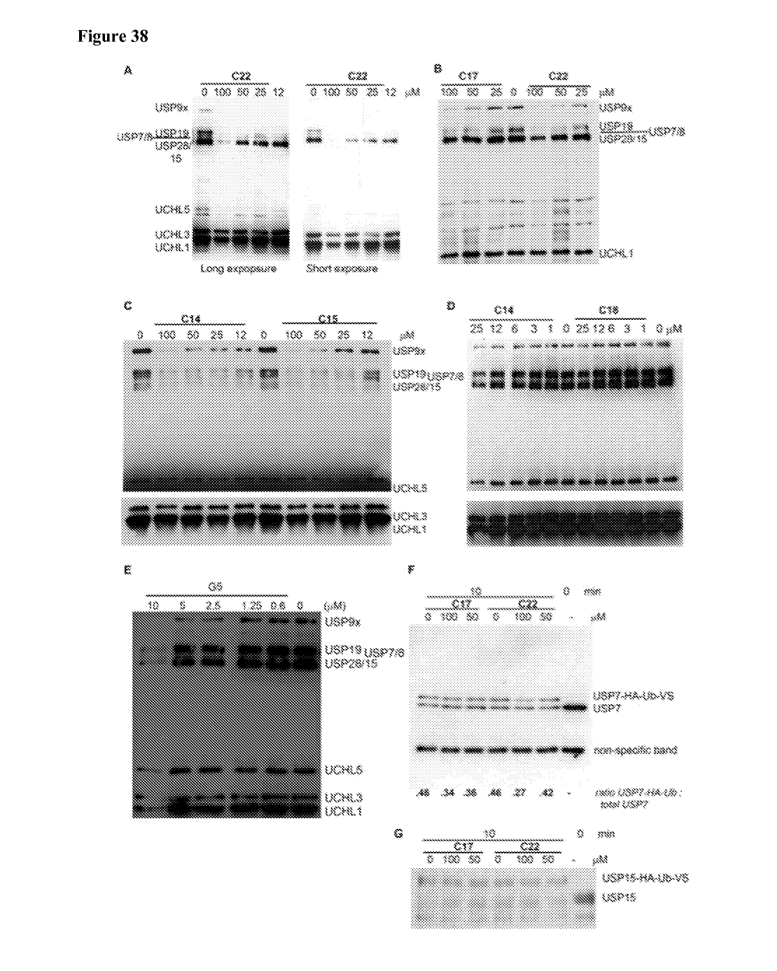

The effect of diphenylcarbonates on HA-Ub-VS labeling was assessed to investigate the selectivity of DUB inhibition. Lysates were preincubated with diphenyl carbonates (75 μM), then treated with HA-Ub-VS (FIG. 31A). The most potent compounds in the HMW-Ub assay, C14, C15, C17, C18 and C22, decreased HA-Ub-VS labeling of several USPs (FIGS. 31 and 38A-D). In contrast, these compounds had relatively little effect on the UCHL enzymes. Dose response curves showed that best compounds, C17 and C22, significantly inhibited the labeling of several high molecular weight proteins at 12 μM. These C17 and C22-sensitive DUBs are most likely USP9x, USP19, USP7/8 and UCHL5, based on molecular weight (FIGS. 31B and 38). The identity of USP7 was confirmed by immunoblotting (FIG. 38F). In contrast, little inhibition of UCH-L1/3/5 was observed below 50 μM. Similar behavior was observed on Cos-1 cell lysates (FIG. 38B).

The kinetics of HA-Ub-VS labeling was examined in order to determine if C17 forms stable DUB complexes as proposed (FIG. 29B). In the absence of C17, eight DUBs were labeled when HEK 293T cell lysates were treated with HA-Ub-VS (FIGS. 31C and D). Labeling was largely complete within 5 min. The presence of C17 (25 μM) was not sufficient to inhibit the labeling of any of the DUBs under these conditions (FIG. 31D), indicating that HA-Ub-VS (1.5 μM) out-competed C17 (25 μM). However, labeling was reduced when lysate was pre-incubated with C17 (250 μM), then diluted 10-fold prior to HA-Ub-VS treatment (FIG. 31C). Thus the C17•DUBs complexes were stable, as expected if thiocarbonylated enzymes formed.

Thiocarbonylated DUBs are expected to hydrolyze, albeit slowly, regenerating active enzymes (FIG. 29A). Indeed, HA-Ub-VS labeling recovered with longer incubation times (FIG. 31C). The labeling of UCH-L5, UCH-L3 and UCH-L1 was recovered within 15 min. However, labeling of USP9x, USP19 and USP7/8 did not recover in 2 h (FIG. 31C). These observations are consistent with the hypothesis that inhibition involves the formation and subsequent decomposition of thiocarbonylated enzymes, and further suggest that the selective inhibition of USPs over UCH-Ls may derive from the stability of their respective thiocarbonylated enzymes.

### Diphenyl Carbonates Inhibit DUBs in Cells

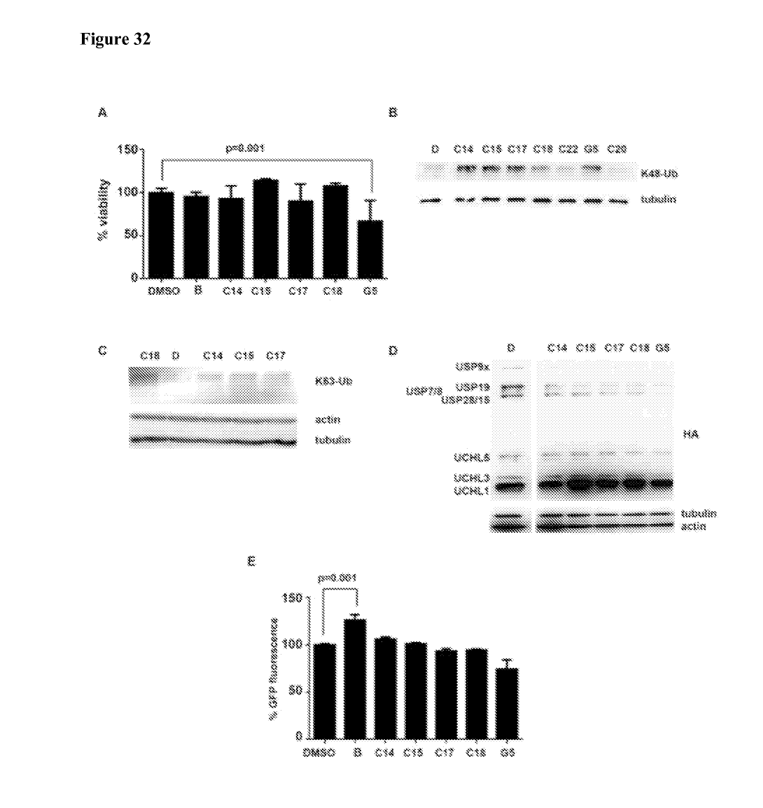

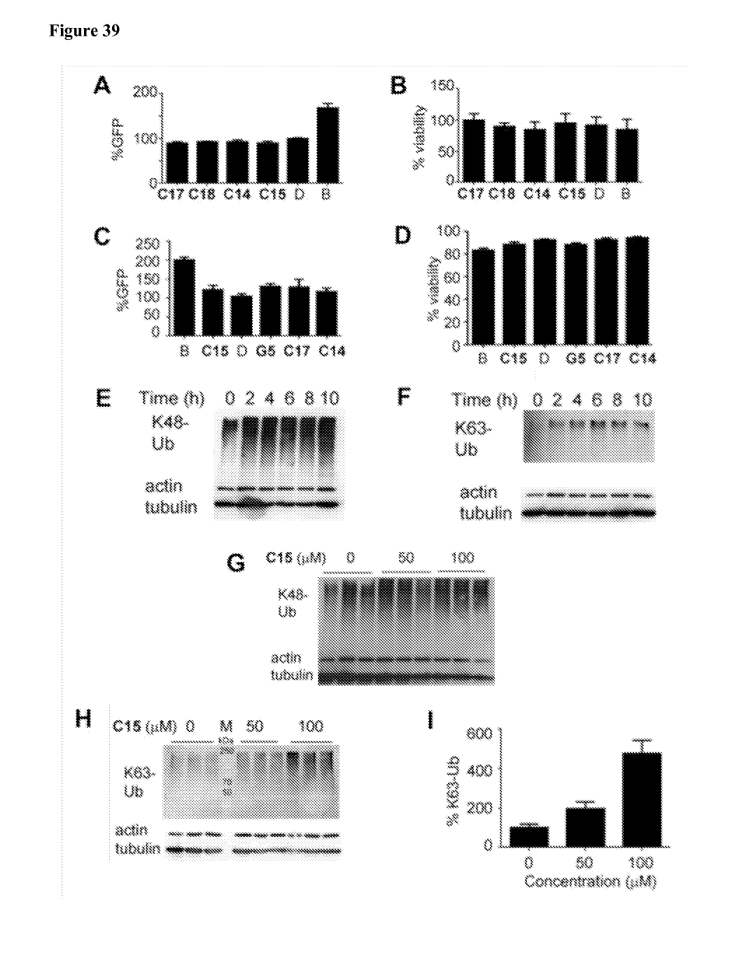

Compounds C14, C15, C17, C18, C20 and C22 (EC₅₀≦50 μM) were candidates for testing in whole cells. The diphenyl carbonates displayed much lower toxicity than the pan-DUB inhibitor G5 in HEK 293T, Cos-1 and CHO cells (FIG. 32A and FIG. 39A-D). Compounds C20 and C22 failed to cause the accumulation of HMW-Ub, suggesting that these compounds were not cell permeable. In contrast, K48-linked HMW-Ub species accumulated when HEK 293T cells were treated with C14, C15, C17 and C18 (FIG. 32B). These compounds also increase the accumulation of K63-linked Ub chains (FIG. 32C).

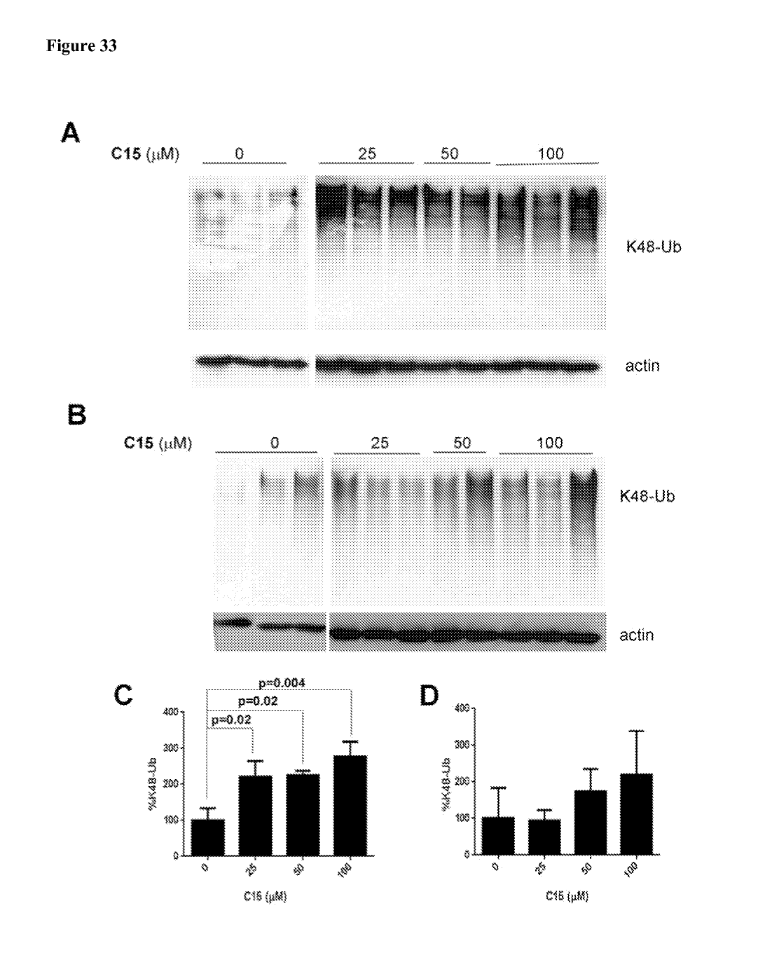

Similar results were obtained in Cos-1 cells. The presence of C15 caused a 3-5-fold increase in total K48-linked and K63-linked HMW-Ub (FIG. 39). Others have reported that pan-DUB inhibitors induce the formation of insoluble K48-linked Ub aggregates. Therefore, the increase of K48-linked ubiquitin in the soluble lysate fraction and whole cell fraction were compared (FIG. 33). A statistically significant increase in K48-linked HMW ubiquitin was only detectable in the soluble fraction (FIG. 33).

Lysates from HEK 293T cells treated with diphenylcarbonates were analyzed by HA-Ub-VS activity profiling to assess the selectivity of DUB inhibition in the context of a cell. All of the compounds decreased the labeling of USP9x and USP7, but had little effect on UCH-L1/3 (FIG. 32D). The most potent compounds were C17 and C18.

The activity of the diphenylcarbonates in the GFP-G76V-Ub assay, which monitors flux through the ubiquitin-proteasome system, was also investigated. The G76V mutation creates an unstable Ub fusion protein that is degraded in a proteasome dependent process. GFP fluorescence increased when HEK 293T cells expressing GFP-G76V-Ub were treated with the proteasome inhibitor bortezomib and decreased upon treatment with G5 (FIG. 39). This decrease has been attributed to increased flux through the ubiquitin-proteasome system triggered by the accumulation of HMW-Ub. Curiously, no change in fluorescence was observed when cells were treated with C14, C15, C17 and C18 (FIG. 32E), even though, as noted above, these compounds caused HMW-Ub to accumulate. Similar effects were observed in Cos-1 cells expressing GFP-G76V-Ub. Perhaps the inability of the diphenylcarbonates to inhibit UCH-L1/3 accounts for the stability of GFP-G76V-Ub. This selectivity might also explain the low cytoxicity of these compounds relative to G5.

### Diphenyl Carbonates Induce the Degradation of Bcr-Abl

Chronic myeloid leukemia and several other blood cancers depend on the oncogenic fusion protein Bcr-Abl kinase for survival. Bcr-Abl has a relatively long lifetime (>24 h) and, like many long-lived proteins, its degradation occurs via an autophagy-mediated process that involves ubiquitination. USP9x removes ubiquitin from Bcr-Abl, preventing degradation. Thus the inhibition of USP9x promotes Bcr-Abl degradation, making USP9x an attractive target for leukemia chemotherapy.

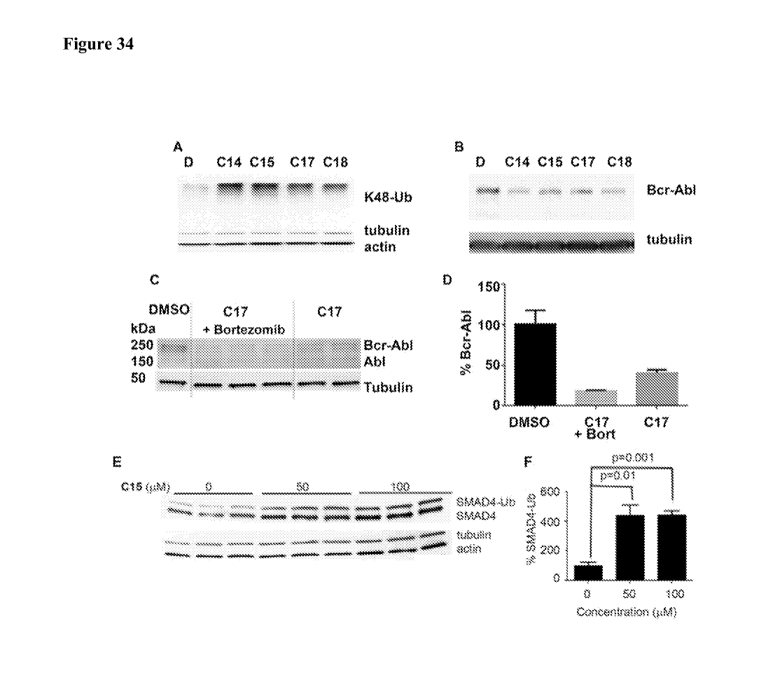

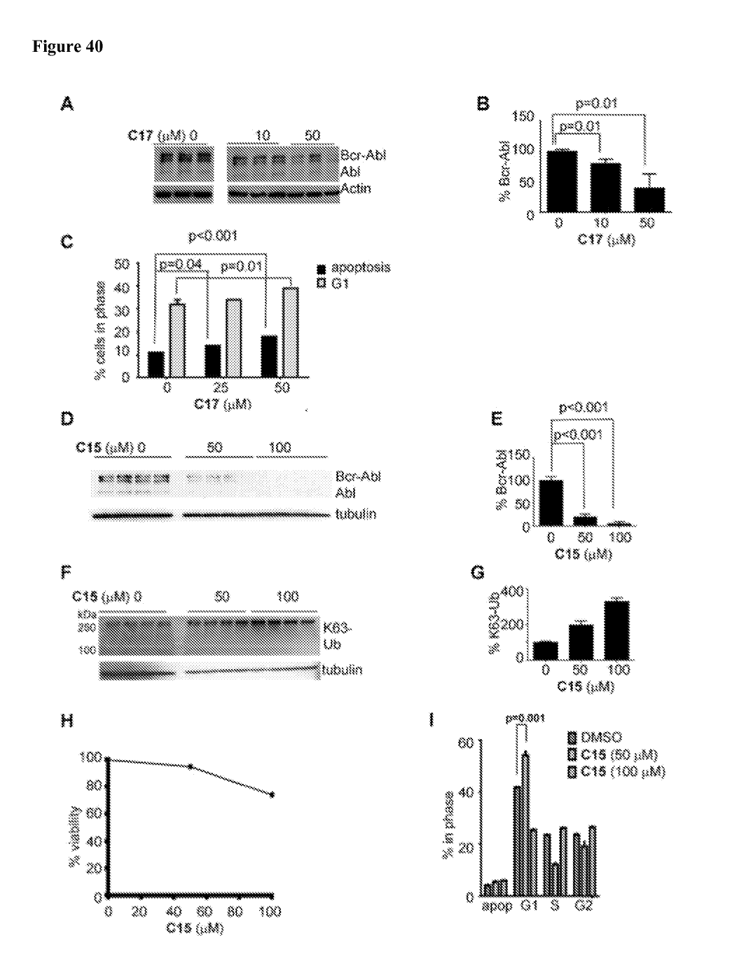

The effect of diphenylcarbonates on K562 leukemia cells was tested. Compounds C14, C15, C17 and C18 caused the accumulation of K48-linked HMW-Ub in K562 cells (FIG. 34A). These compounds also caused a decrease in the levels of Bcr-Abl, consistent with USP9x inhibition (FIGS. 34B and 40). The decrease in Bcr-Abl was dose-dependent (FIG. 40A,B). The presence of bortezomib did not prevent Bcr-Abl degradation, as expected for an autophagy-mediated process (FIG. 34C,D). C17 also caused a dose-dependent increase in G1 and apoptotic cells after a 24 h incubation (FIG. 40C). A similar, dose dependent decrease in Bcr-Abl was observed with C15 treatment (FIG. 40D,E). Also, C15 caused the accumulation of K63-linked ubiquitin (FIG. 40F,G). Like C17, C15 caused a decrease in viability, and increase in G1 and apoptotic cells at 50 μM (FIG. 40H,I).

The semi-selective USP9x inhibitor WP1130 also causes a decrease in the levels of soluble Bcr-Abl. However, this decrease is accompanied by an increase of Bcr-Abl in insoluble protein aggregates. In contrast, the samples used in these experiments were prepared with sonication in SDS to solubilize protein aggregates prior to PAGE analysis. Therefore the decrease in Bcr-Abl levels cannot be attributed to sequestration into insoluble aggregates, and must instead result from an increase in degradation. The different consequences of treatment with diphenylcarbonates or WP1130 suggest may derive from differences in their mechanism of action or target repertoire.

USP9x also regulates the ubiquitination and localization of the signaling protein SMAD4. Treatment with C15 increased SMAD4 monoubiquitination (FIG. 34E,F), further demonstrating that diphenyl carbonates block USP9x functions in whole cells.

### Diphenyl Carbonates Stabilize p53

The ability of diphenyl carbonates to inhibit USP7 function in cells was also assessed. The ubiquitin-ligase Mdm2 is a substrate for USP7. Mdm2 is responsible for the ubiquitination and subsequent degradation of p53. Mdm2 is over-expressed in many cancer cells, resulting in the depletion of p53. Mdm2 is itself degraded via an ubiquitin-dependent process. USP7 removes ubiquitin from Mdm2, protecting it from degradation. Like Mdm2, USP7 is over-expressed in many cancers. Inhibition of USP7 promotes the proteasome-mediated degradation of Mdm2, which causes an increase in p53 levels, as well as those of the downstream signaling protein p21/WAF1, and ultimately induces apoptosis.

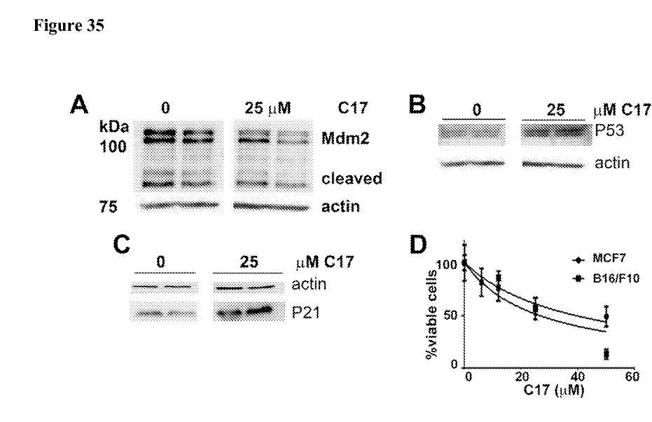

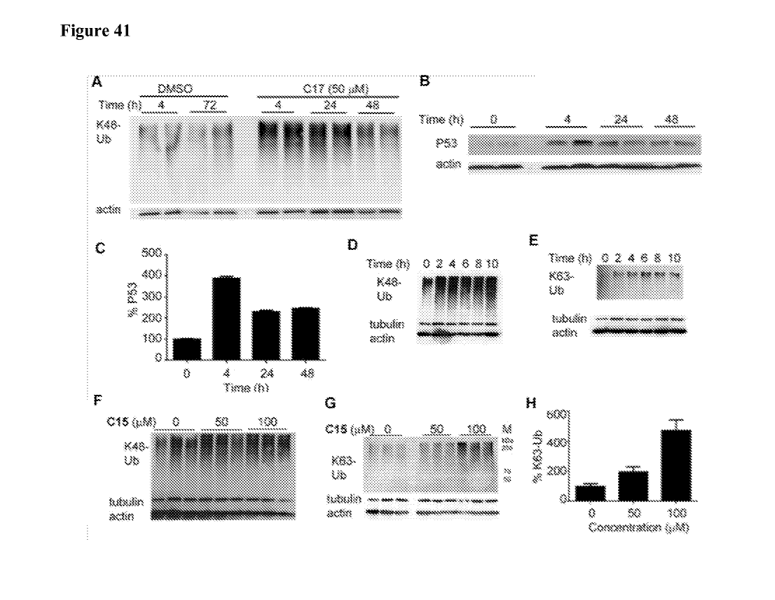

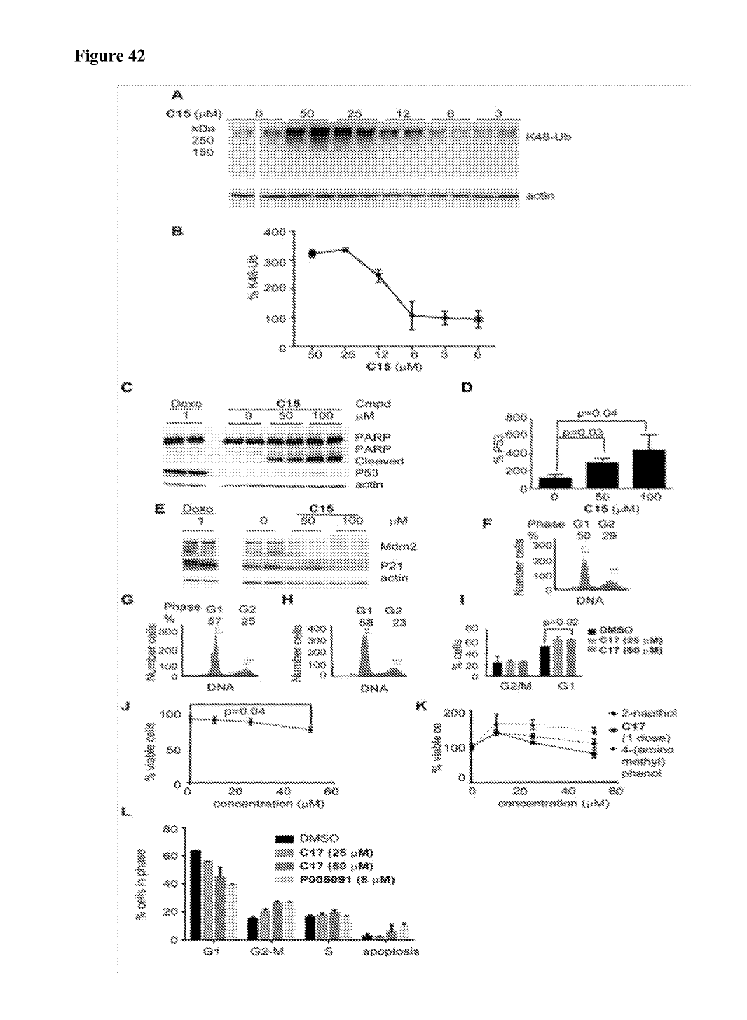

The effects of diphenyl carbonates on MCF7 breast cancer cells that express wild-type P53 but downregulate its expression through Mdm2 were tested. As observed in other cell lines, C17 caused the sustained accumulation of soluble HMW-Ub (FIG. 41A). Gratifyingly, Mdm2 levels decreased with a concomitant increase in p53 (FIG. 35A,B and FIG. 41B,C). A robust increase in p21/WAF1 levels was also observed (FIG. 35C). Likewise, C15 increased soluble K48 and K63 linked ubiquitin (FIG. 41D-H) and also decreased Mdm2 and increased P53 levels (FIG. 42C,D). Curiously, this compound decreased p21/WAF1 (FIG. 42E).

### C17 Inhibits Growth and Induces Apoptosis in Cancer Cells Suppressing p53 Levels Via Mdm2

The increase in p53 levels observed when MCF7 cells were treated with C15 and C17 should lead to G1 arrest and growth inhibition. To test this hypothesis, MCF7 cells were treated with C17 every 24 h for a total of 72 hours (approximately 2 cell cycles in MCF7 cells). \Cell viability decreased by 50% (FIG. 35D). FACS analysis revealed that C17 induced a significant increase in G1 phase cells (FIG. 42F-I). When MCF7 cells were treated with a single dose of C17, a small, but significant, decrease in viability was observed after 24 h (FIG. 42J). However, no cytotoxicity was observed after 72 h after a single dose of C17 (FIG. 42K). These observations suggest C17 was not stable under these conditions. 2-Naphthol and aminomethylphenolhydrolysis products of C17, were not cytotoxic (FIG. 42K). These observations demonstrate that the cytotoxic effects of C17 are reversible.

Compound C17 also inhibited proliferation in B16/F10 cells, a melanoma cell line that suppresses p53 via Mdm2-mediated degradation (FIG. 35D). Interestingly in the case of B16/F10 cells, both the USP7 specific inhibitor P005091 and C17 showed a decrease in cells in G1 phase and an increase in G2 (FIG. 42L). Collectively, these results demonstrate that C17 inhibited USP7 in cells.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula I, Formula II, Formula III, or Formula IV: 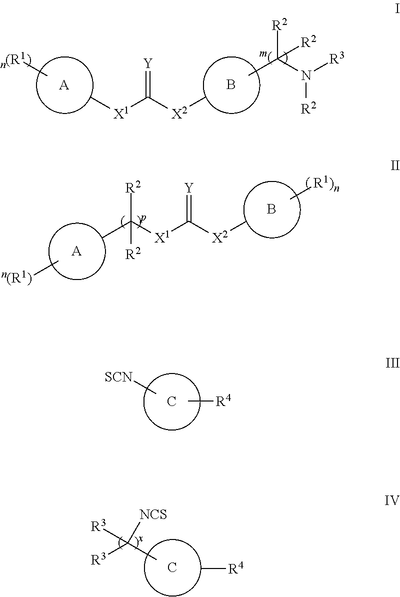
or a pharmaceutically acceptable salt thereof,
wherein, independently for each occurrence, 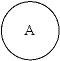
is optionally substituted aryl or optionally substituted heteroaryl; 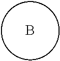
is optionally substituted aryl or optionally substituted heteroaryl; 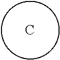
is aryl or heteroaryl;
n is 0, 1, 2, or 3;
R¹ is halo, optionally substituted alkyl, —OSO₂R², —OSO₃H, —OC(O)R², —ONO₂, —OP(O)(OR²)₂, alkoxy, or aryloxy;
R² is —H, optionally substituted alkyl, optionally substituted aryl, or optionally substituted heteroaryl;
R³ is —H, optionally substituted alkyl, optionally substituted aryl, optionally substituted heteroaryl, optionally substituted aralkyl, optionally substituted heteroaralkyl, —C(O)R², or —C(O)OR²;
R⁴ is absent, or is optionally substituted aminoalkyl, cyano, halo, optionally substituted alkyl, optionally substituted amino, or nitro;
X¹ is O, S, or NR²;
X² is O, S, or NR²;
Y is O, S, or NR²;
m is 1, 2, or 3;
p is 0, 1, 2, or 3; and
x is 3, 4, or 5,
provided the compound is not

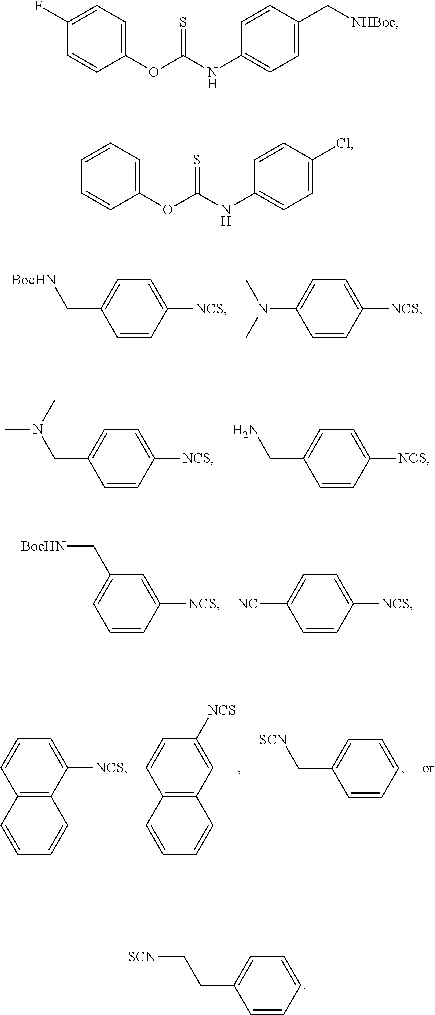

2-3. (canceled)

4. The compound of claim 1, wherein the compound is a compound of Formula I or a compound of Formula II; and 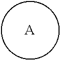
is optionally substituted naphthyl or optionally substituted phenyl.

5-8. (canceled)

9. The compound of claim 1, wherein the compound is a compound of Formula I or a compound of Formula II; n is 1; and 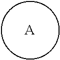
is para-substituted phenyl.

10-12. (canceled)

13. The compound of claim 1, wherein the compound is a compound of Formula I or a compound of Formula II; and 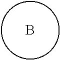
is optionally substituted phenyl.

14. (canceled)

15. The compound of claim 1, wherein the compound is a compound of Formula I or a compound of Formula II; and 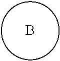
is para-substituted phenyl.

16. (canceled)

17. The compound of claim 1, wherein the compound is a compound of Formula I or a compound of Formula II; and n is 1.

18. The compound of claim 1, wherein the compound is a compound of Formula I or a compound of Formula II; and m is 1.

19. (canceled)

20. The compound of claim 1, wherein the compound is a compound of Formula I or a compound of Formula II; and R² is —H.

21-27. (canceled)

28. The compound of claim 1, wherein R³ is —C(O)OR² or an optionally substituted benzyl.

29-38. (canceled)

39. A compound selected from the group consisting of:

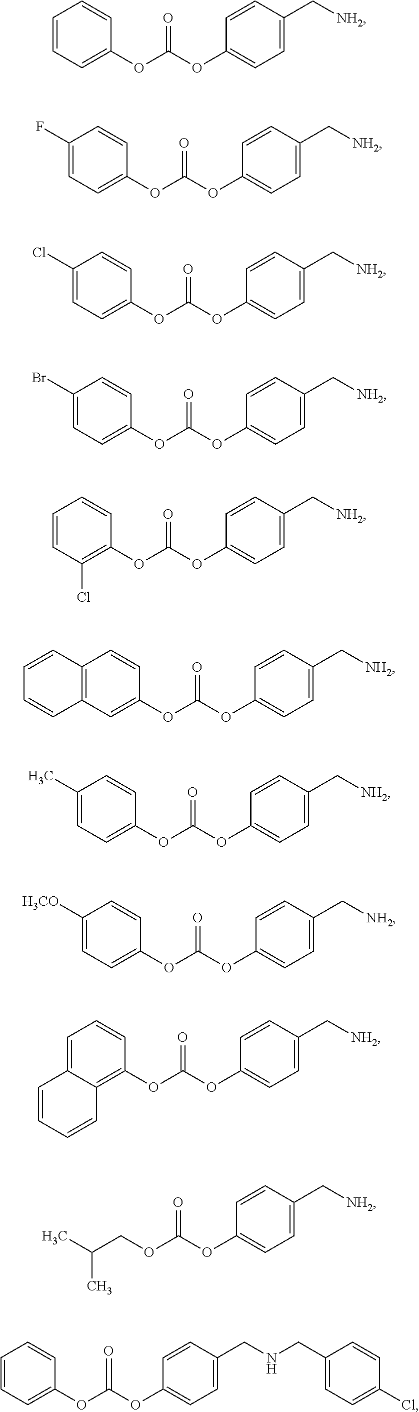

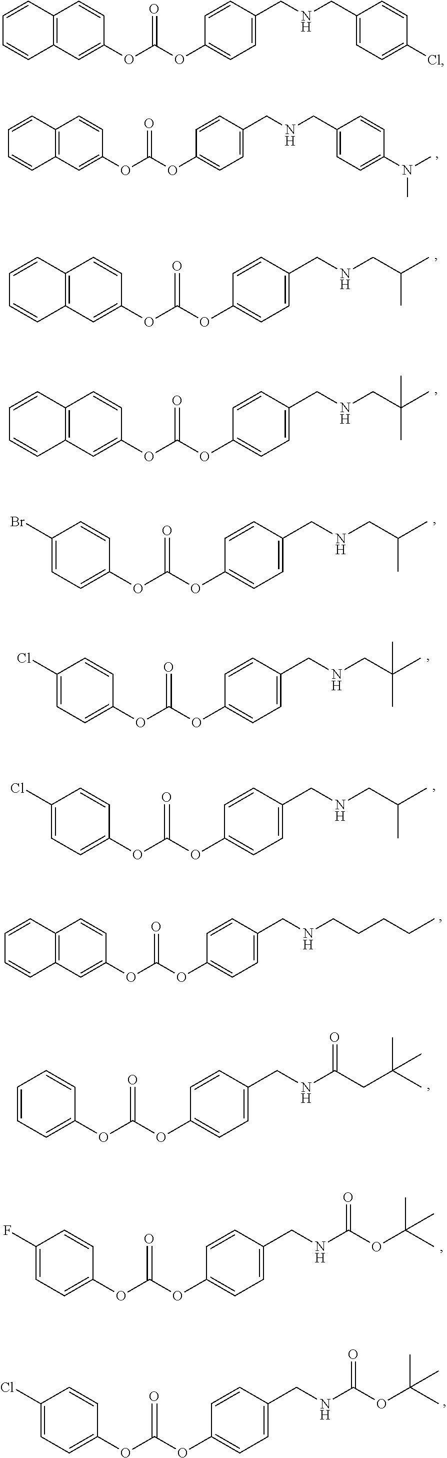

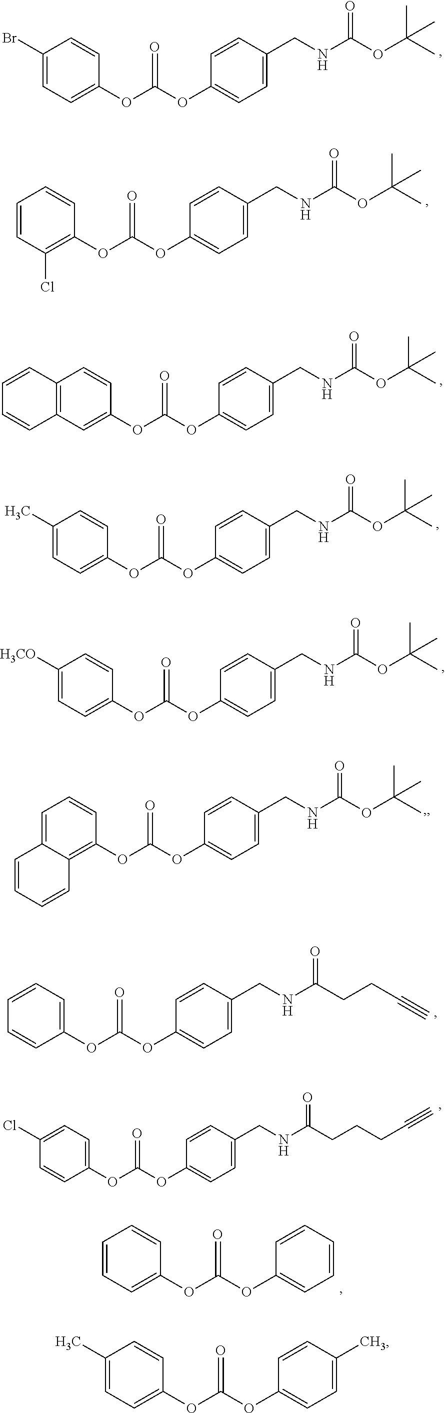

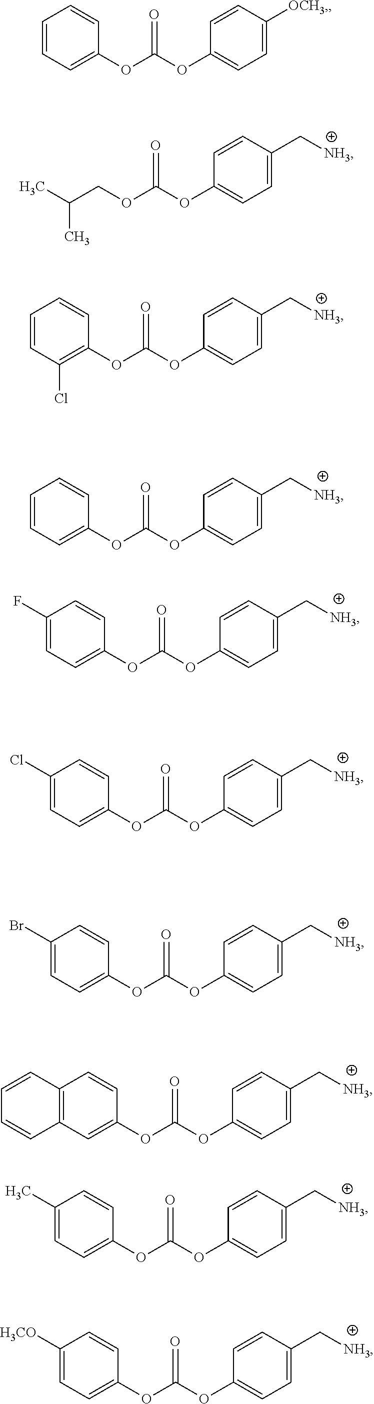

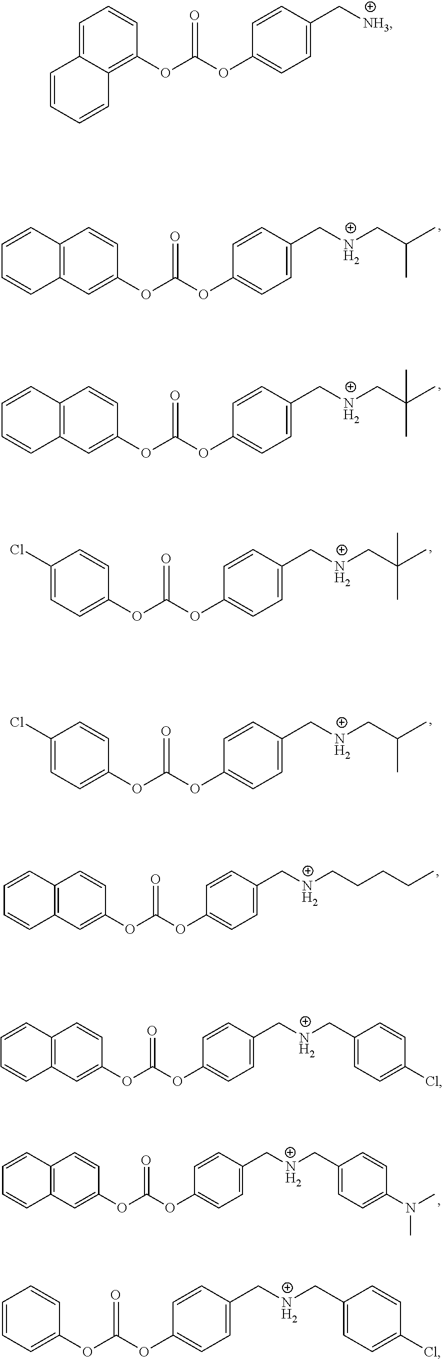

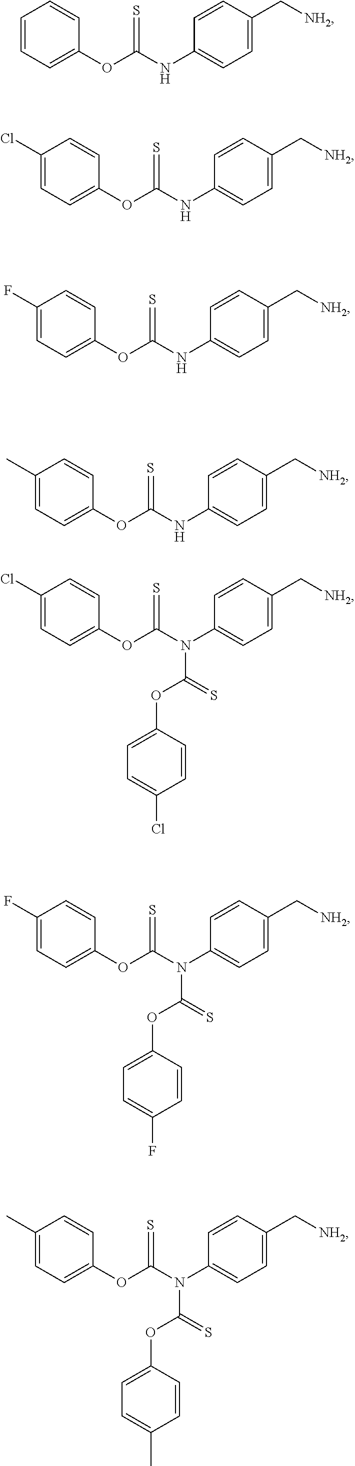

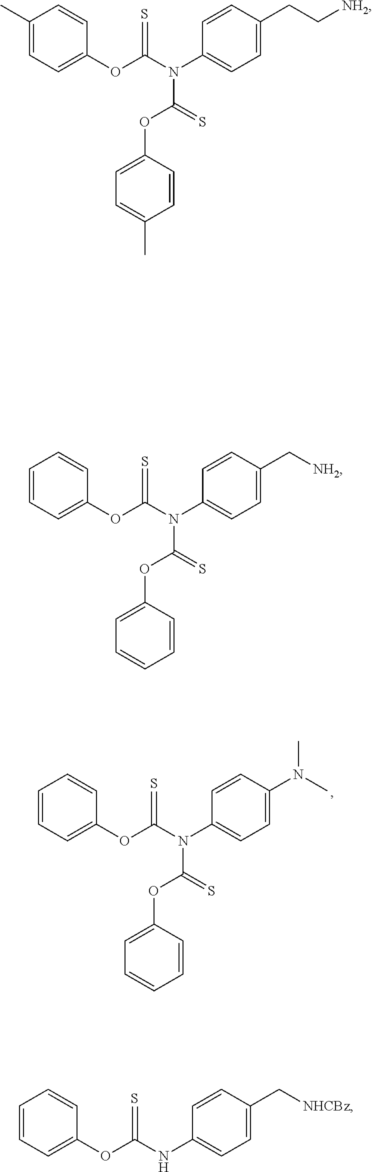

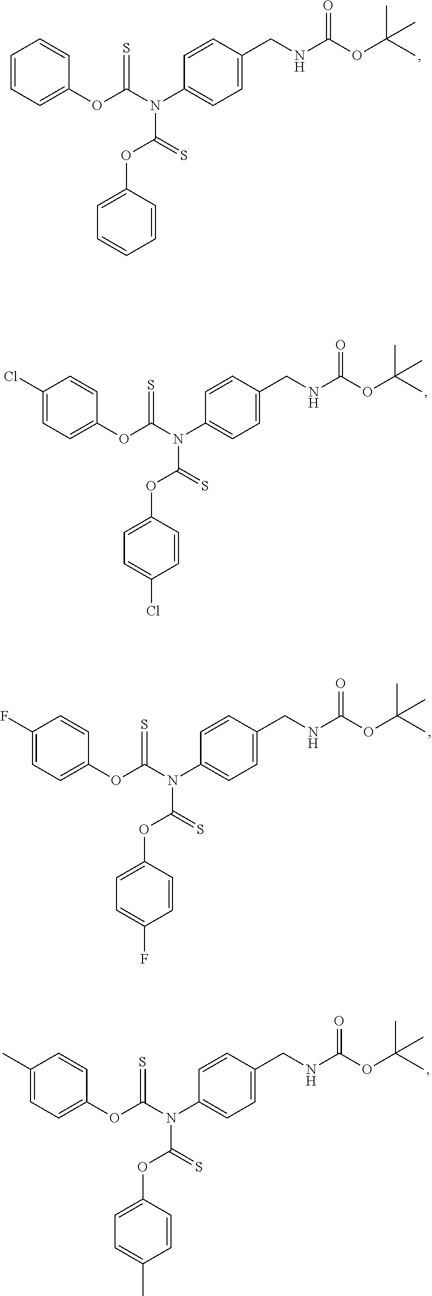

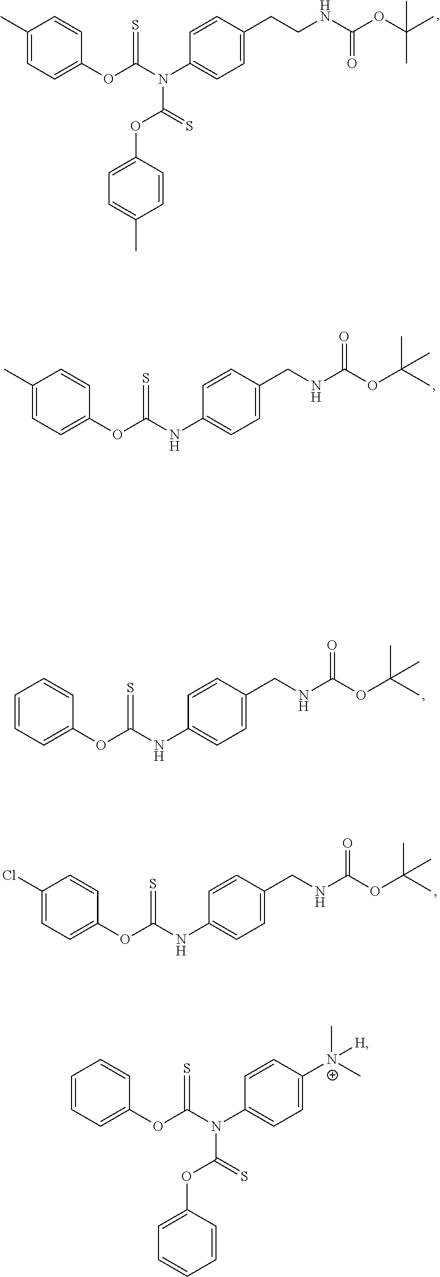

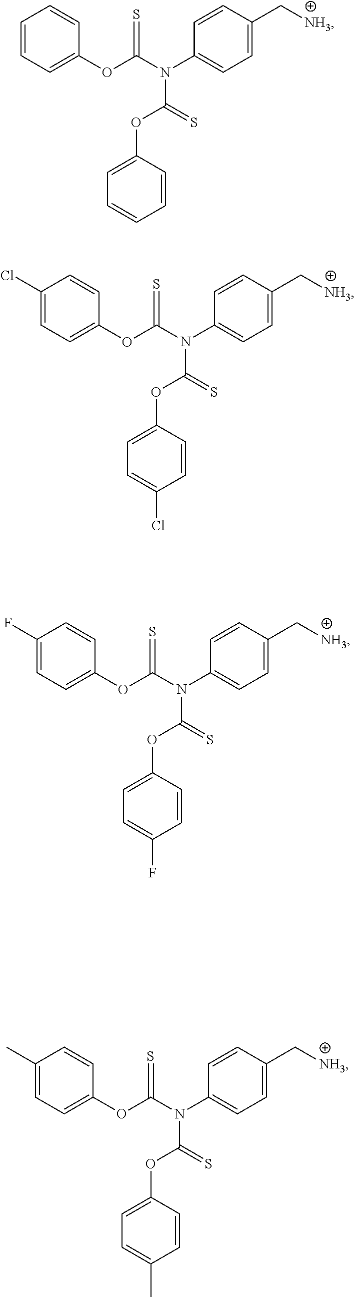

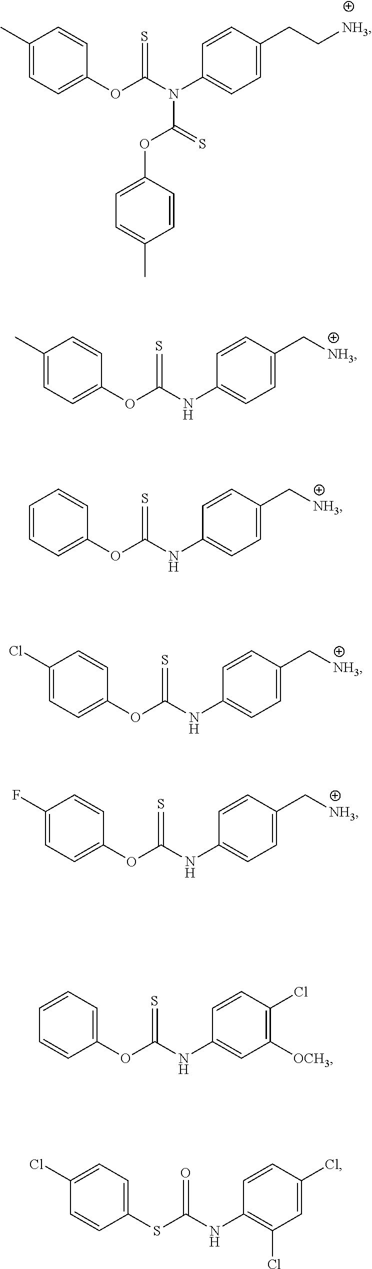

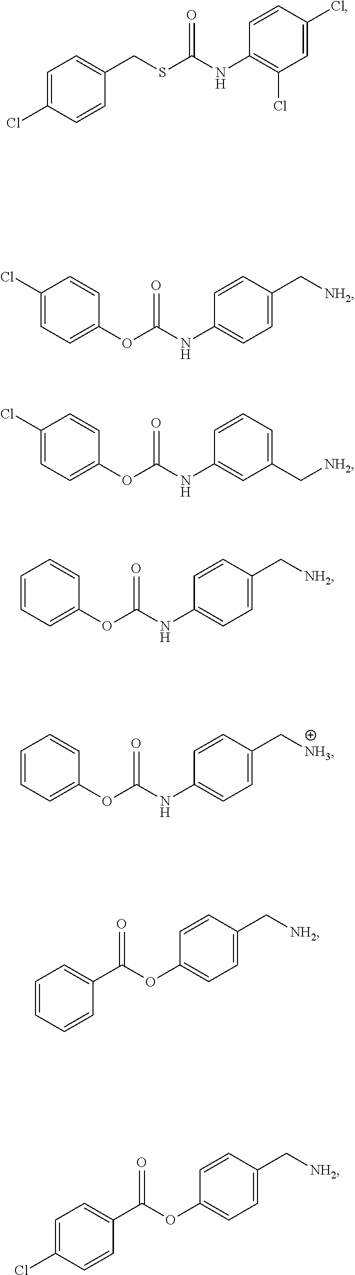

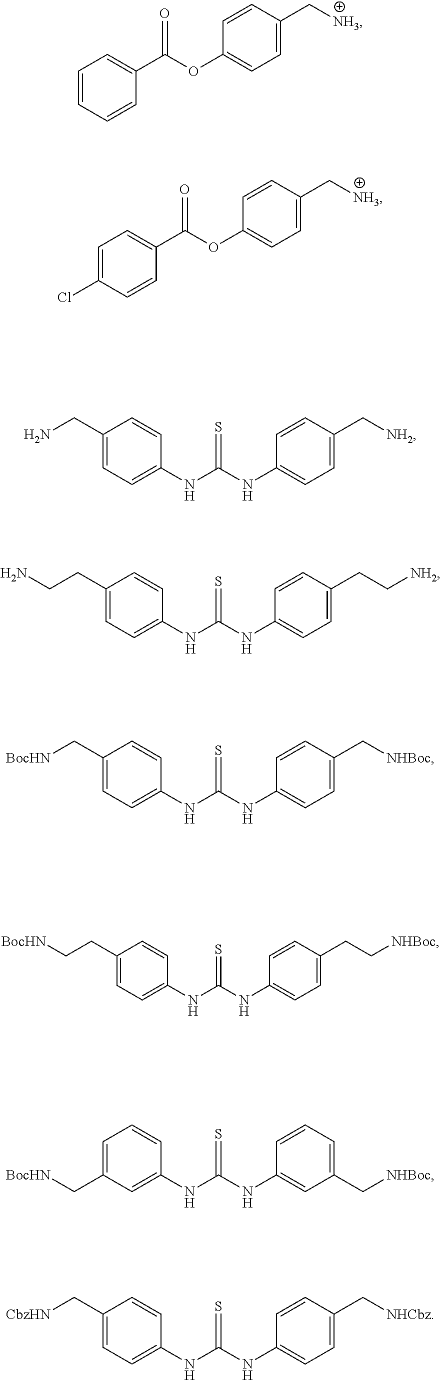

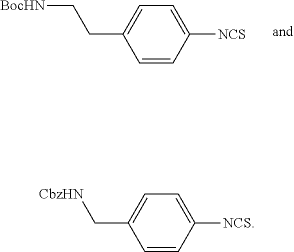

40-46. (canceled)

47. The compound of claim 1, wherein the compound is a compound of Formula III or a compound of Formula IV; and 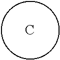
is phenyl or naphthyl.

48-53. (canceled)

54. A pharmaceutical composition comprising a pharmaceutically acceptable carrier, and
(i) a compound of Formula I or Formula II;
(ii) a compound of Formula III or Formula IV; or
(iii) a compound selected from the group consisting of

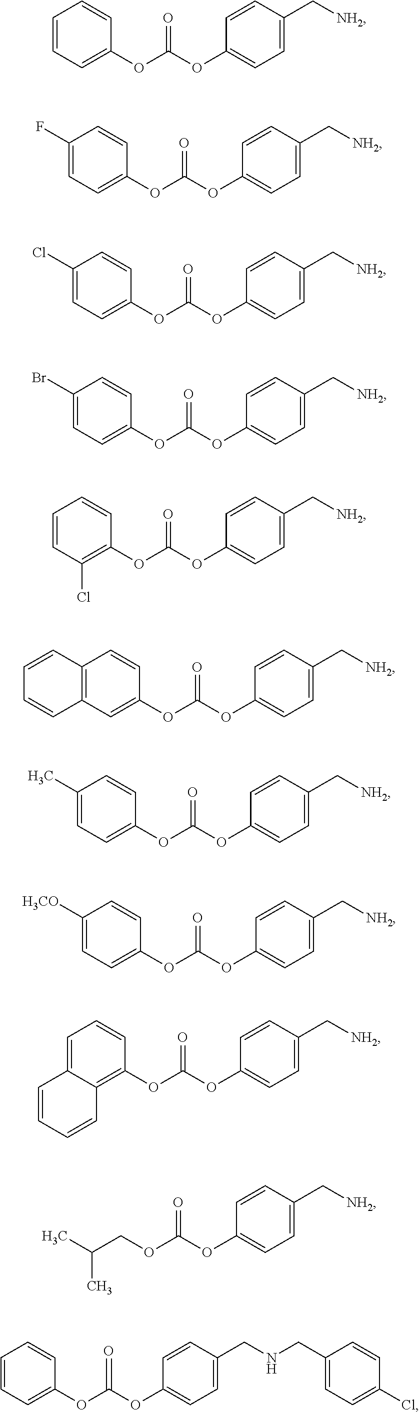

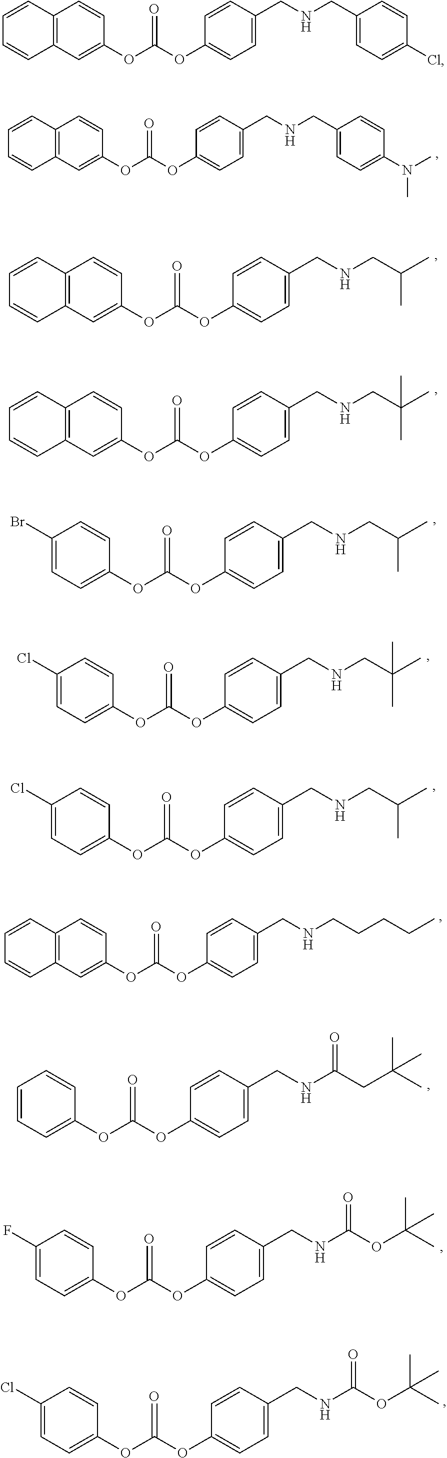

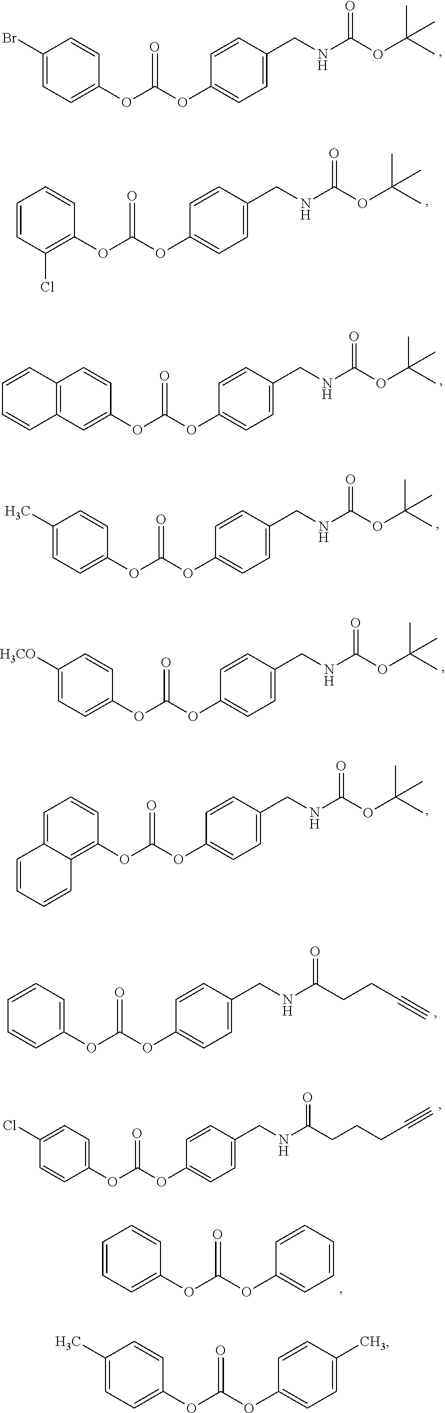

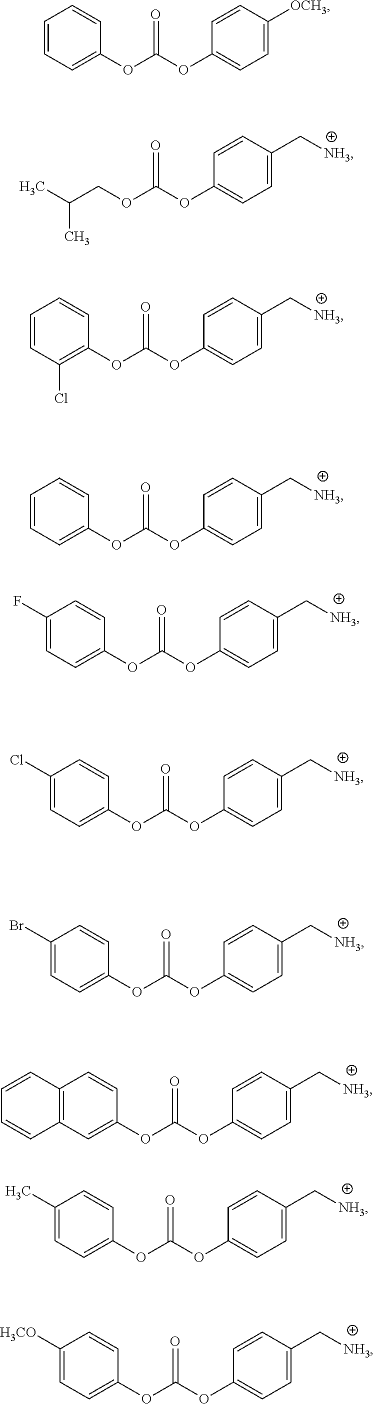

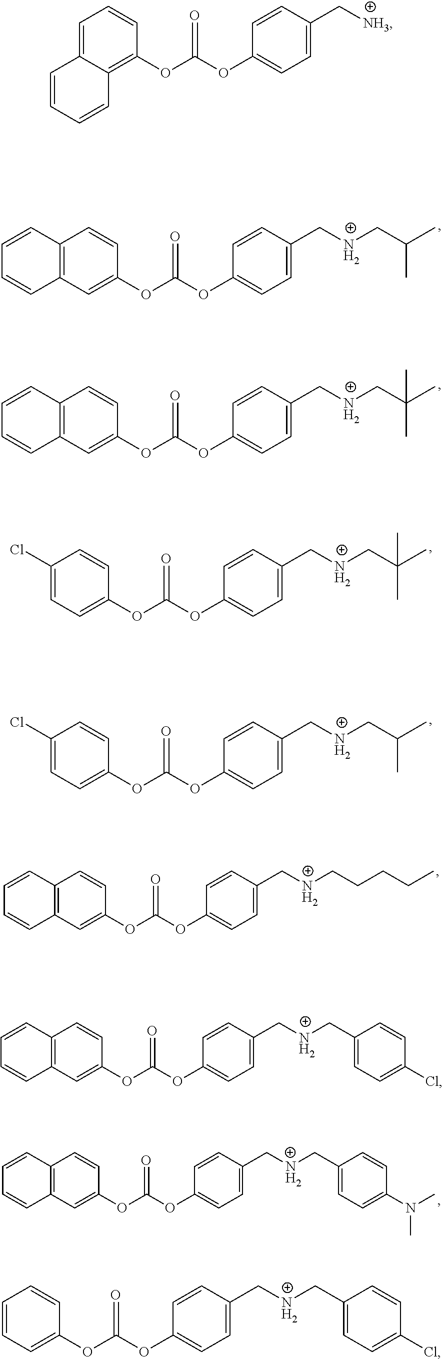

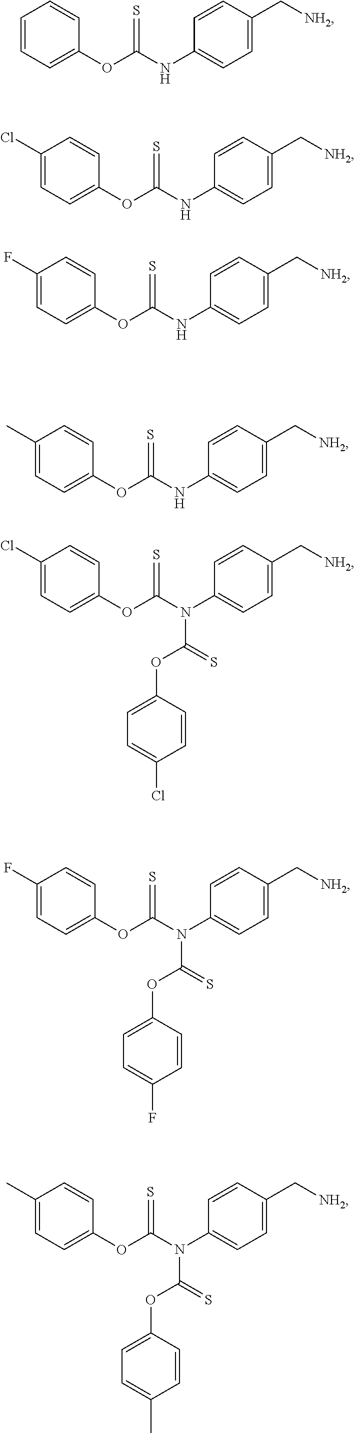

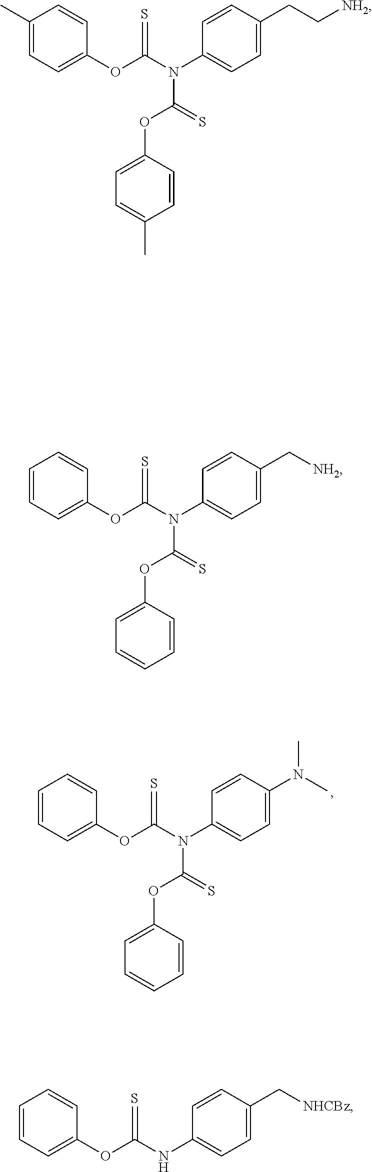

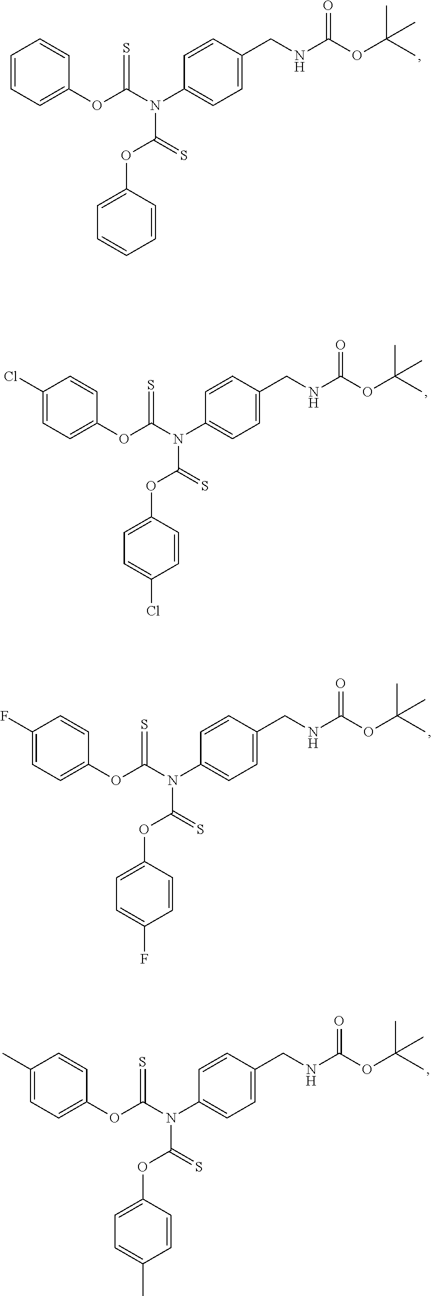

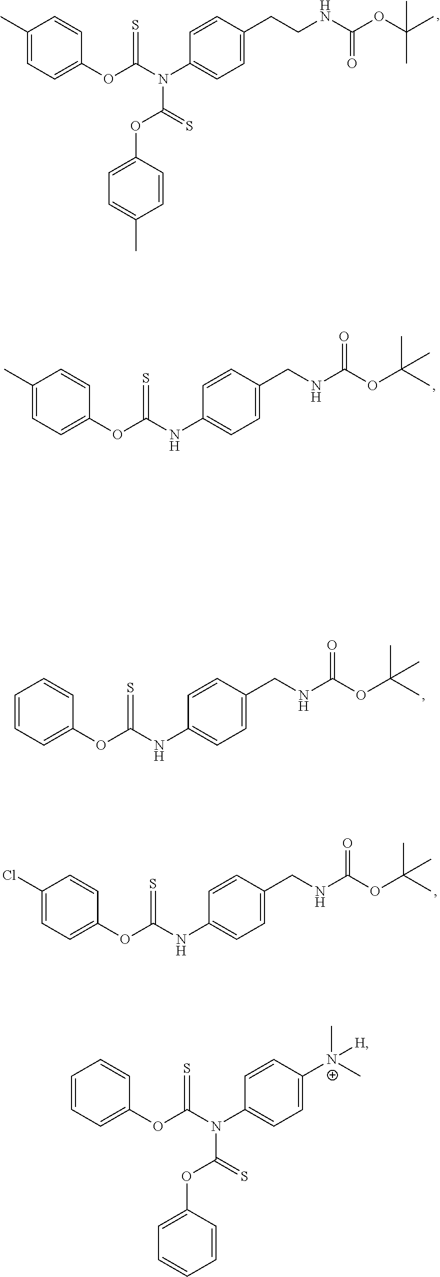

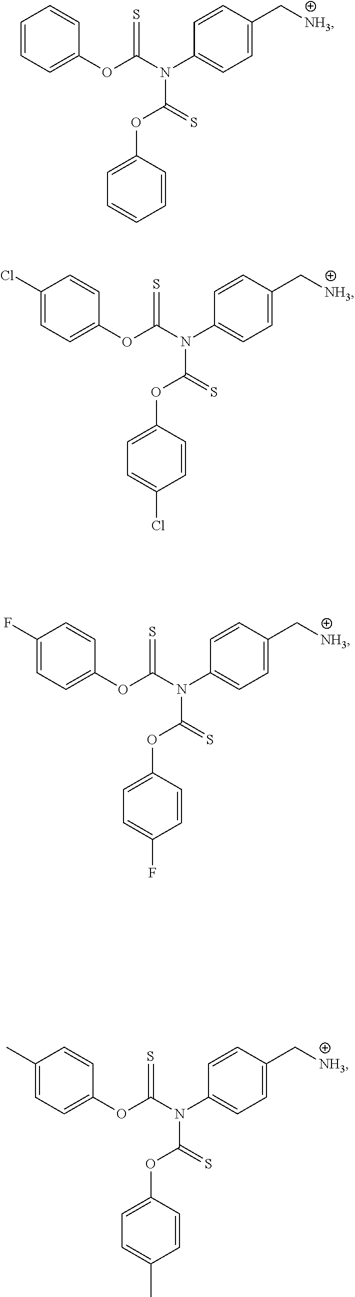

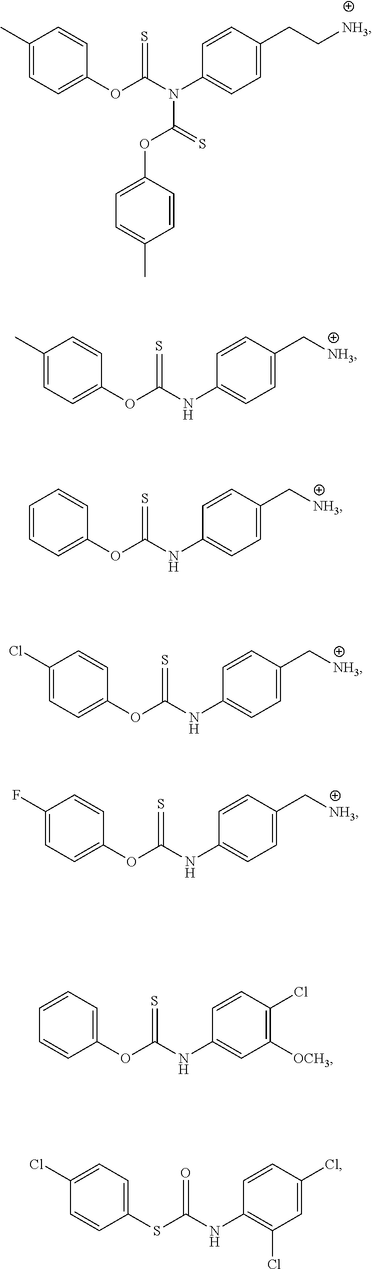

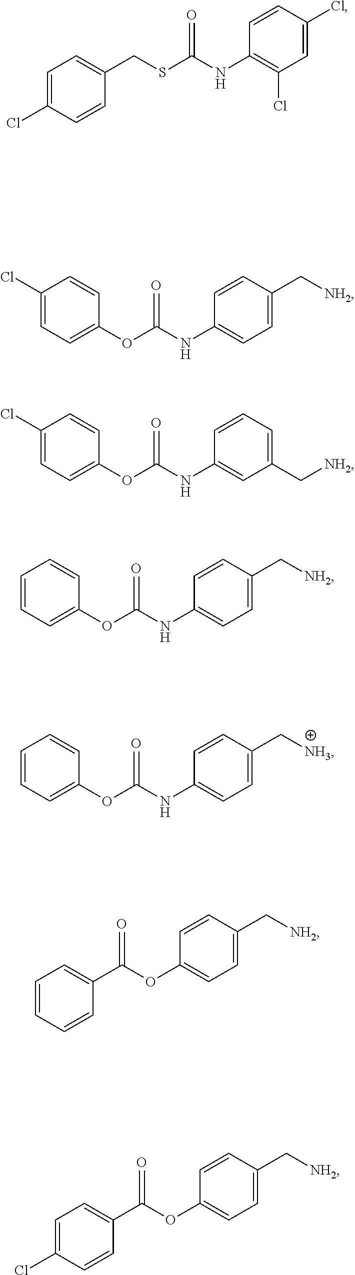

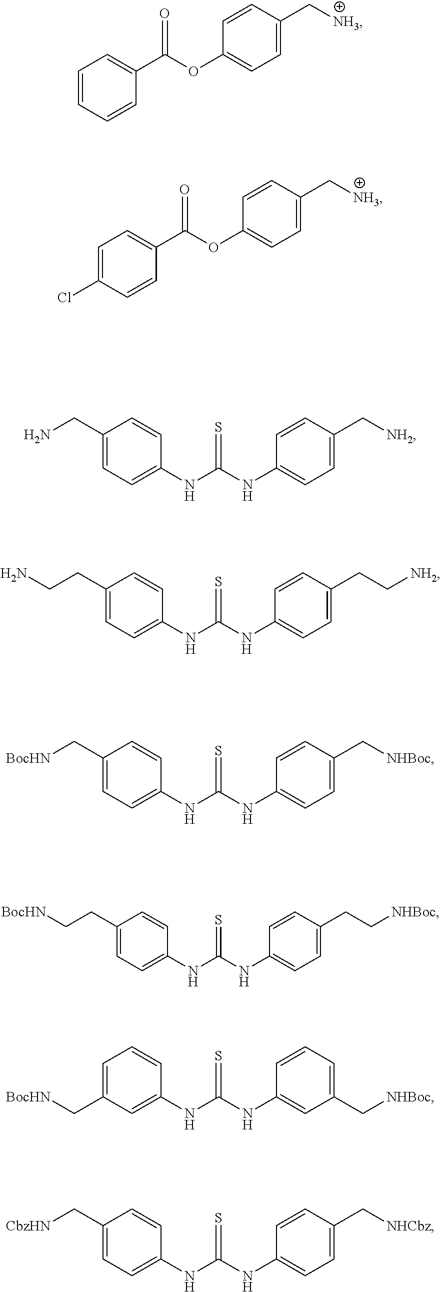

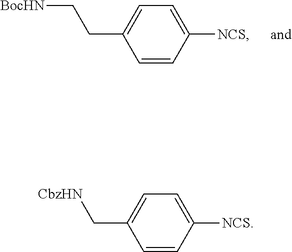

55. A method of preventing or treating a proteinopathy, a cell proliferative disorder or disease, or an infection, comprising the step of: administering to a subject in need thereof a therapeutically effective amount of
(i) a compound of Formula I or Formula II;
(ii) a compound of Formula III or Formula IV;
(iii) a compound selected from the group consisting of 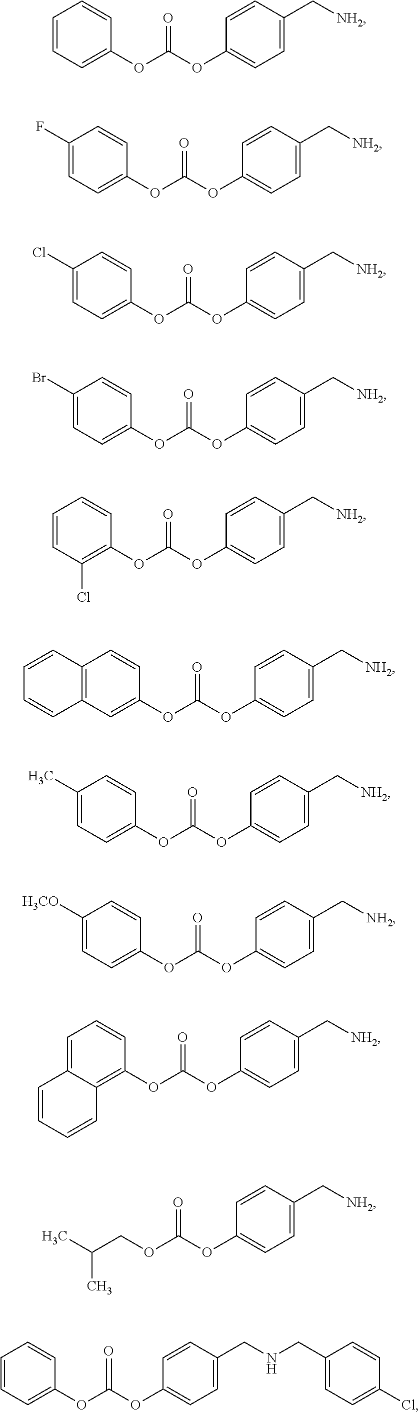 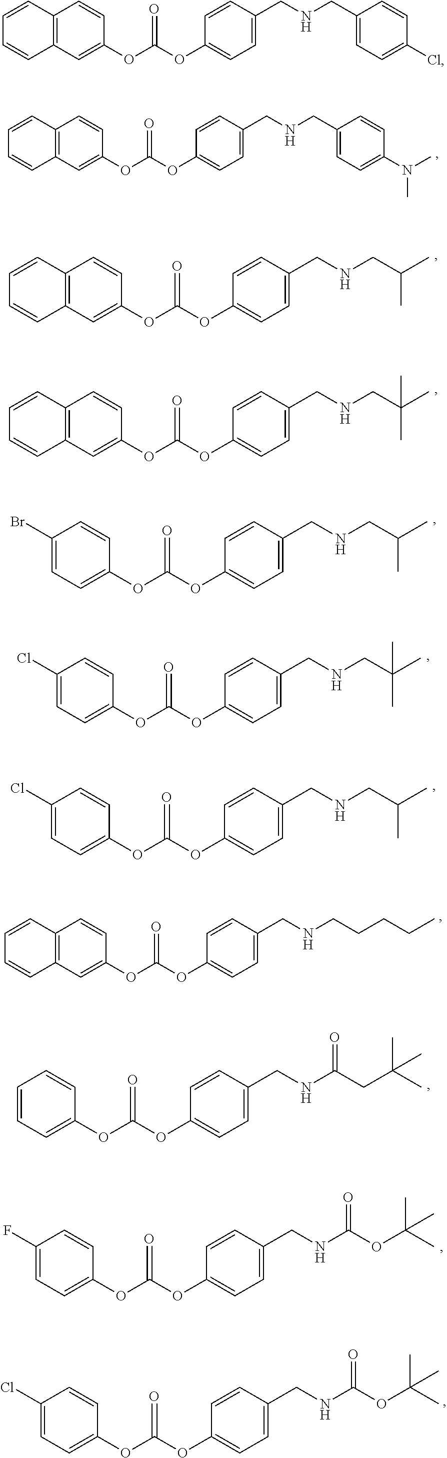 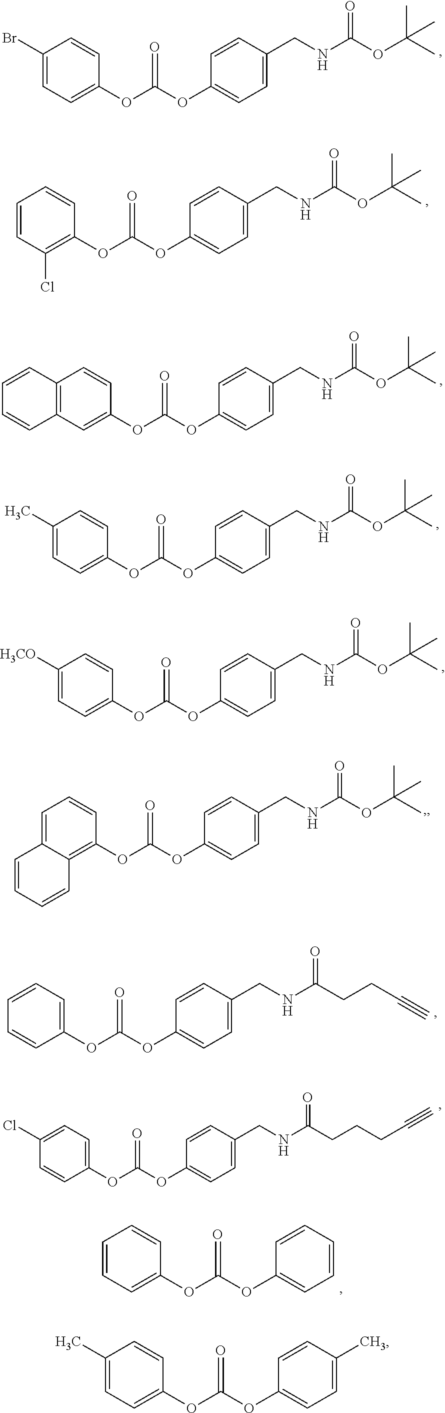 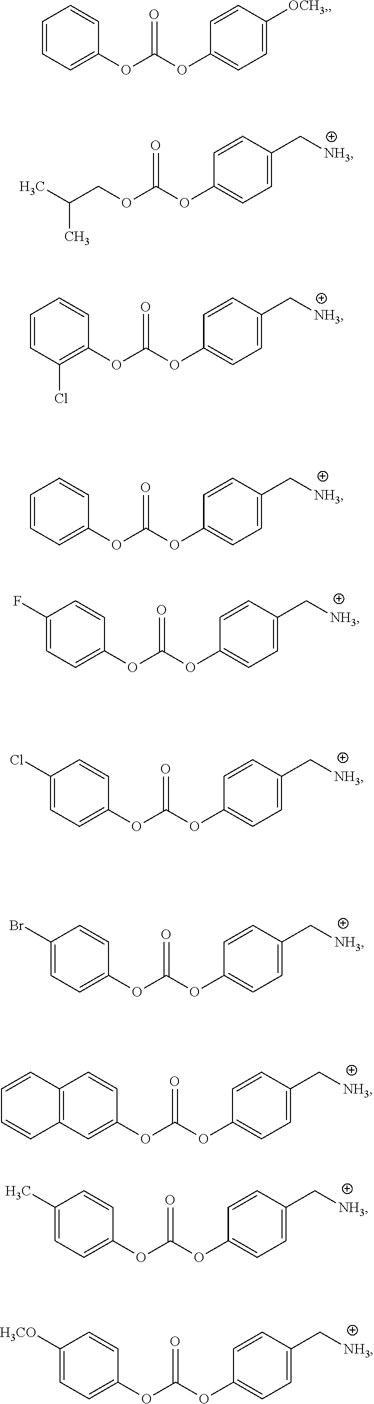 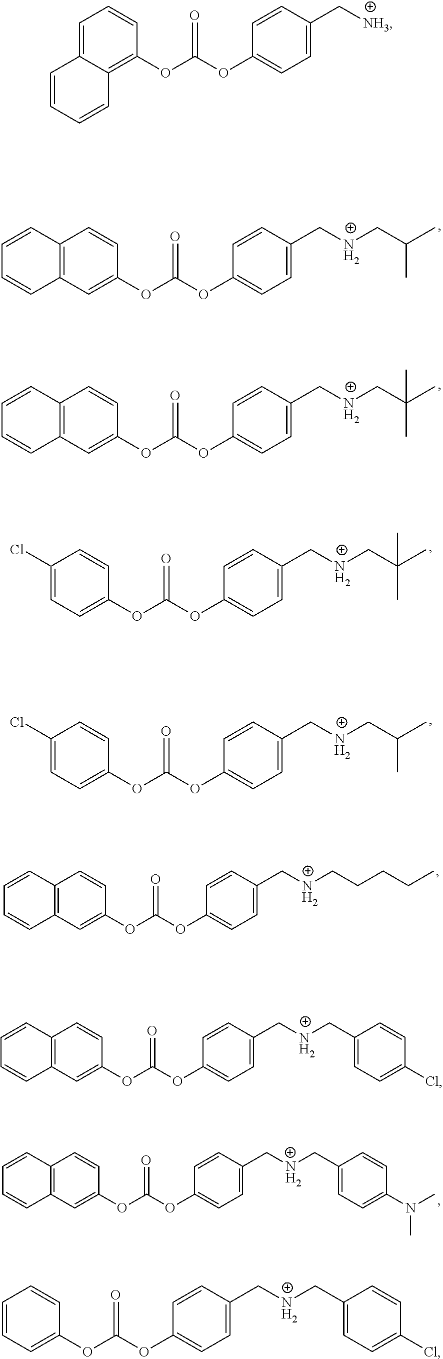 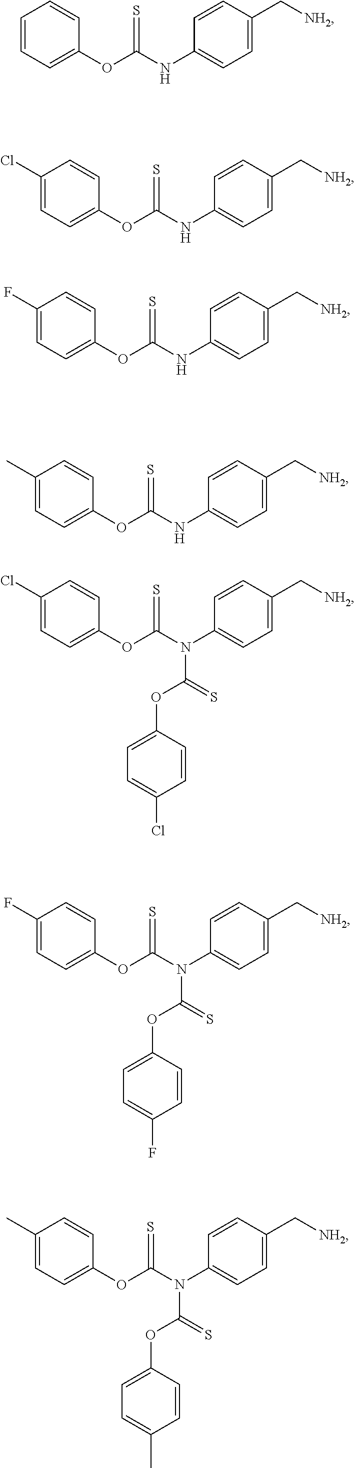 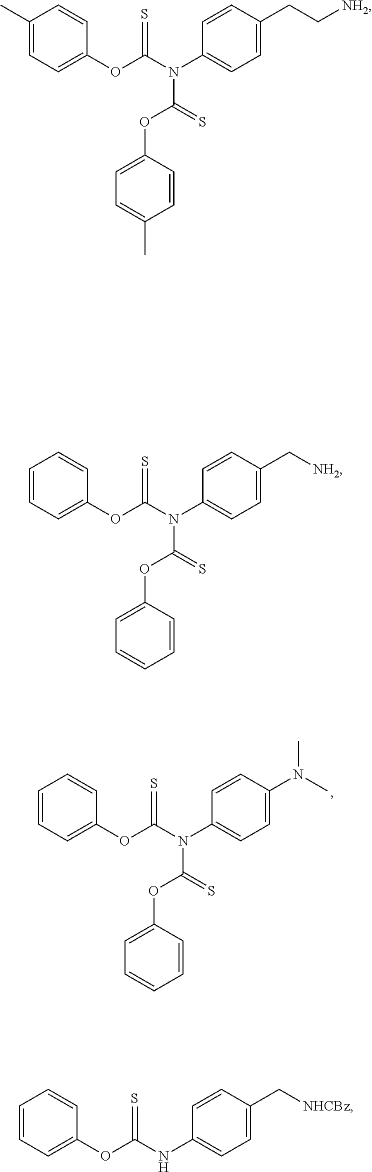 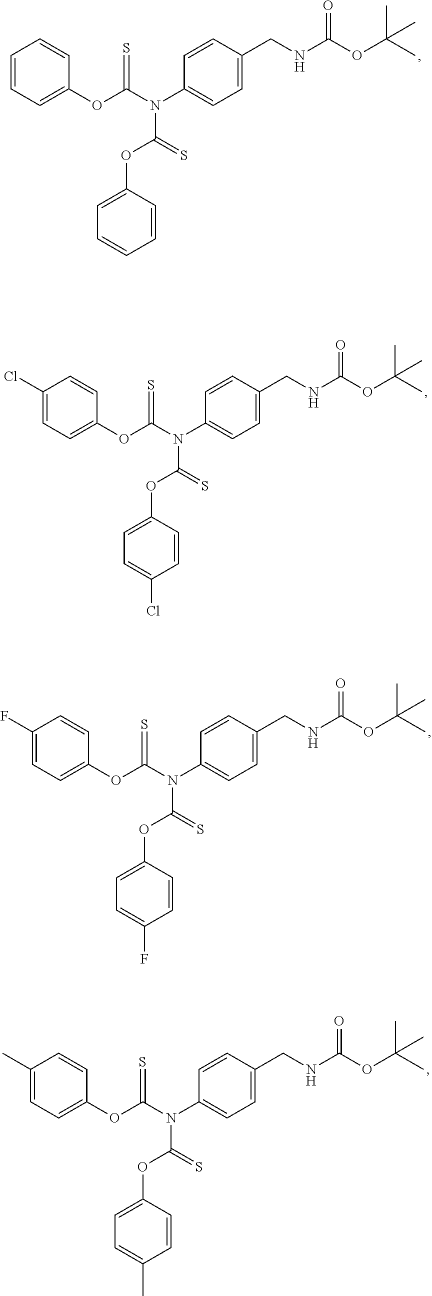 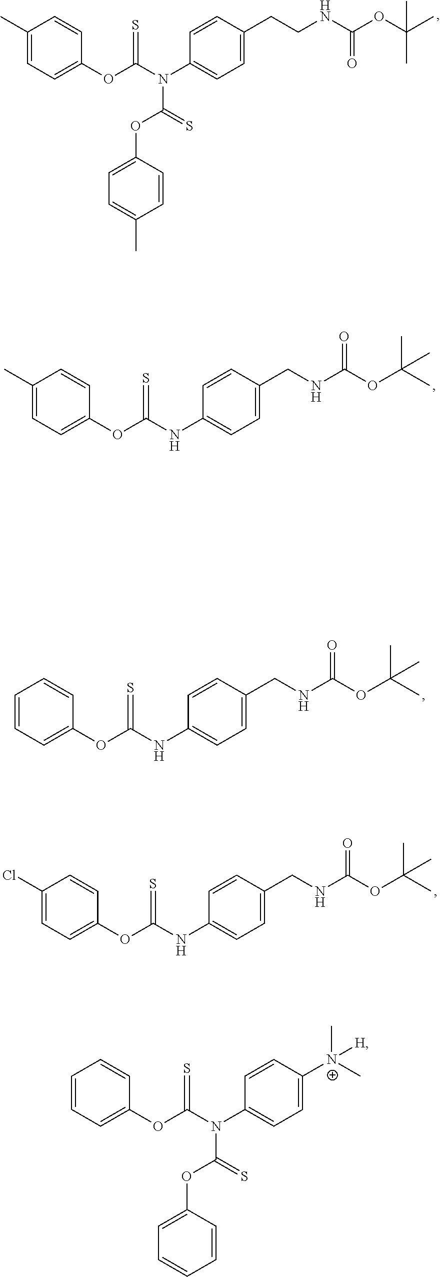 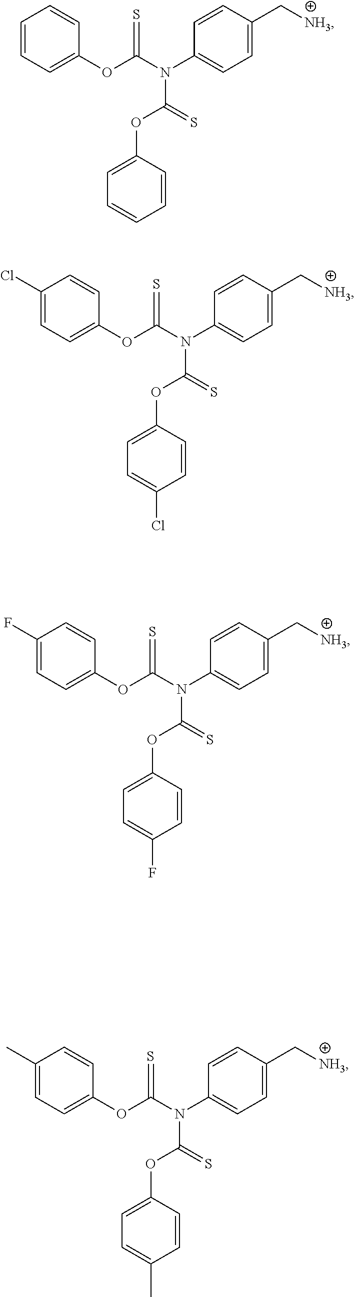 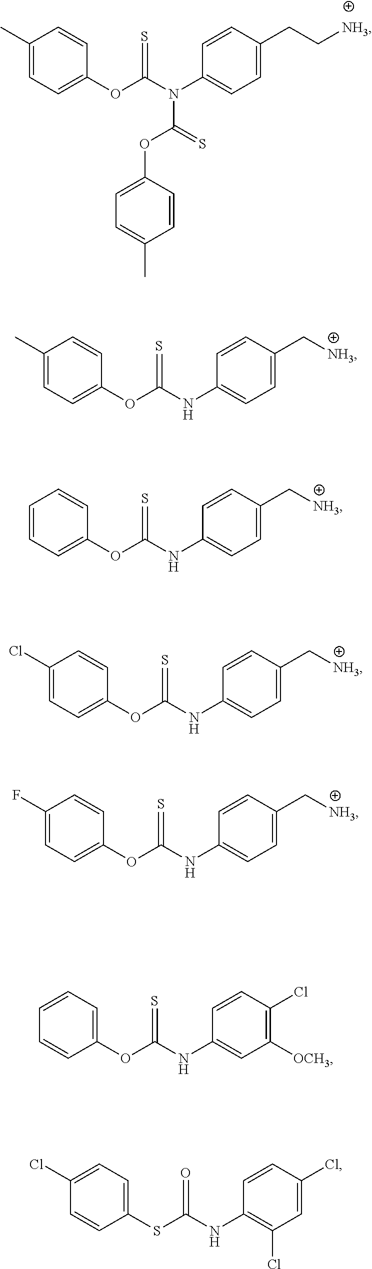 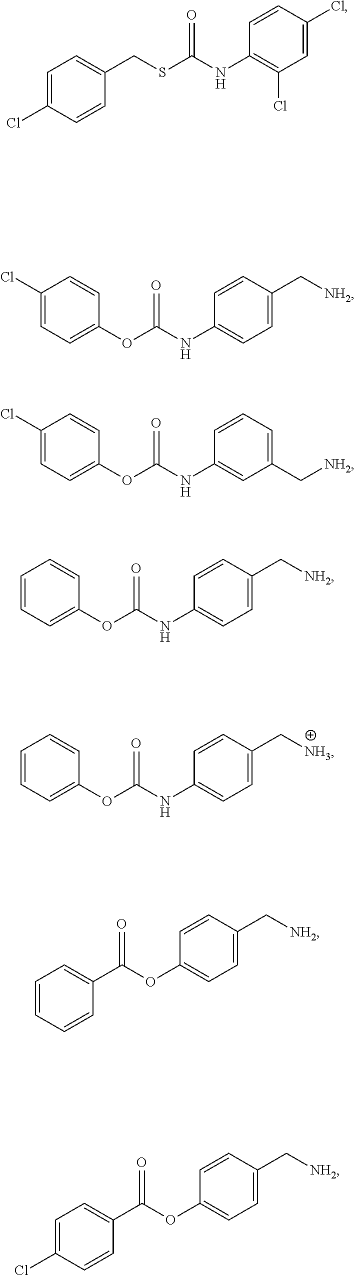 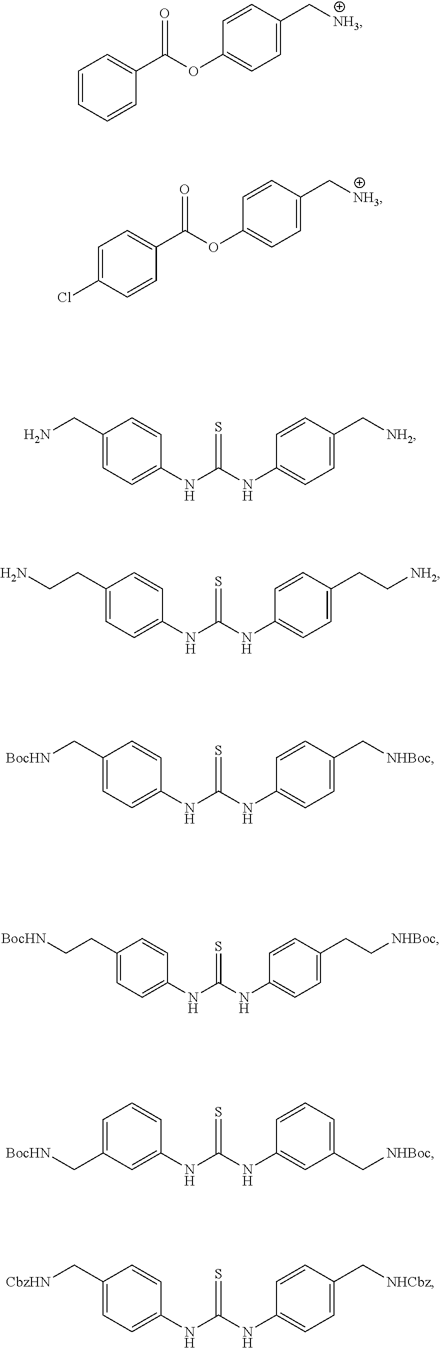 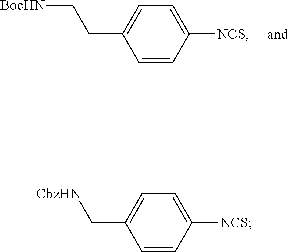
or
(iv) a compound selected from the group consisting of

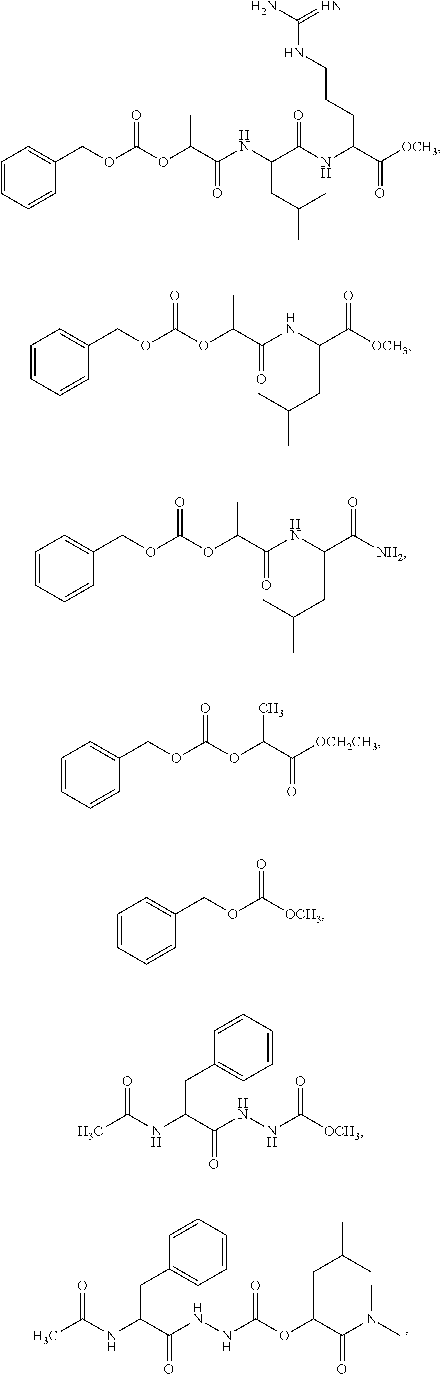

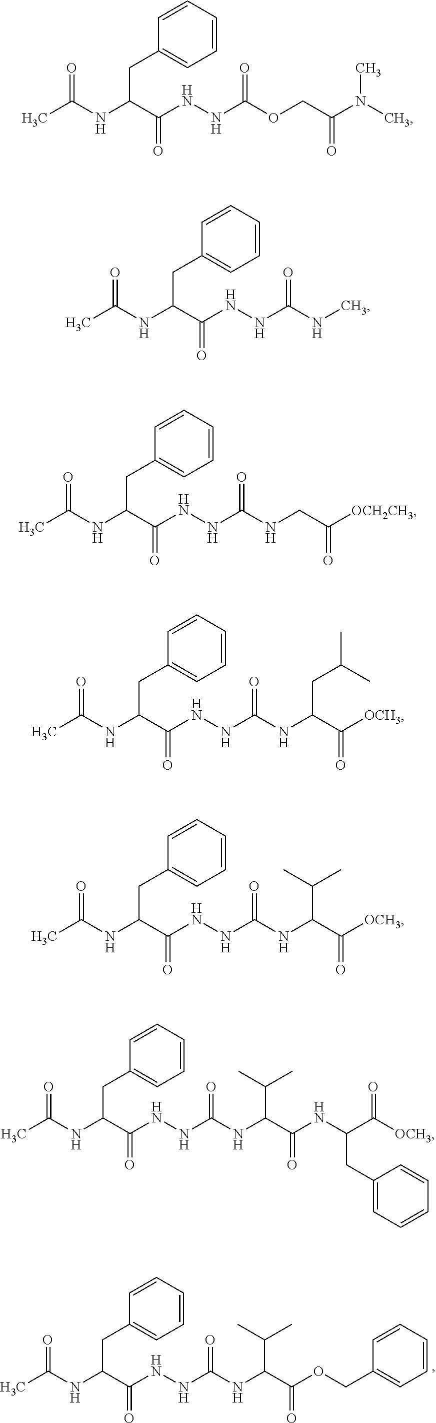

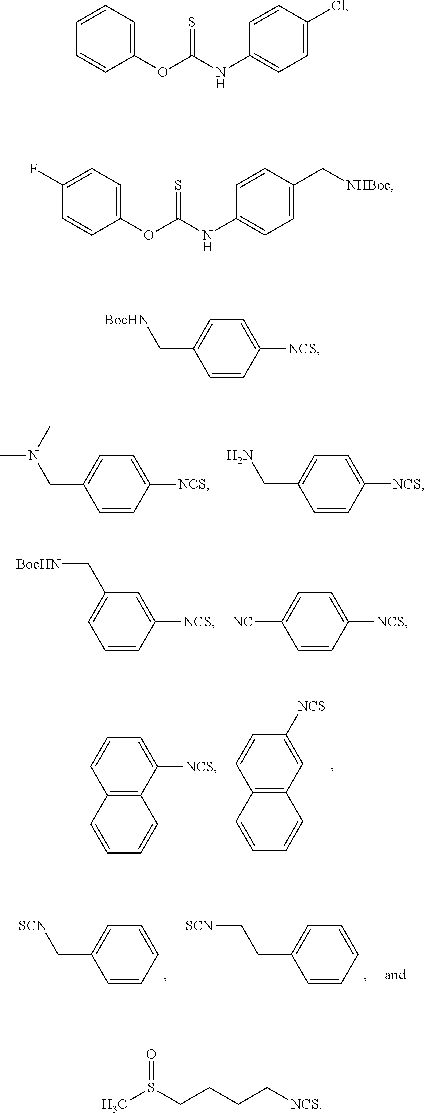

56-69. (canceled)
